(19) United States
(12) Patent Application Publication
(10) Pub. No.: US 2023/0405040 A1
(43) Pub. Date: Dec. 21, 2023

(54) METHODS FOR TREATING ATHEROSCLEROTIC CARDIOVASCULAR DISEASE WITH LPA-TARGETED RNAi CONSTRUCTS

(71) Applicant: AMGEN INC., Thousand Oaks, CA (US)

(72) Inventors: Winnie SOHN, Oak Park, CA (US); Zachary JONES, Santa Monica, CA (US); Helina KASSAHUN, Rockville, MD (US)

(73) Assignee: AMGEN INC., Thousand Oaks, CA (US)

(21) Appl. No.: 18/035,198

(22) PCT Filed: Nov. 4, 2021
(86) PCT No.: PCT/US2021/058012

§ 371 Date: May 3, 2023

Related U.S. Application Data
(60) Provisional application No. 63/110,309, filed on Nov. 5, 2020.

(51) Int. Cl. A61K 31/713; C12N 15/113; A61K 47/54; A61K 9/00; A61P 3/06

(52) U.S. Cl. CPC A61K 31/713; C12N 15/113; A61K 47/549; A61K 9/0012; A61P 3/06

(57) ABSTRACT

The present invention relates to methods for treating or preventing atherosclerotic cardiovascular disease and other conditions associated with elevated levels of lipoprotein (a) (Lp(a)) using RNAi constructs targeting the LPA gene, which encodes apolipoprotein(a), a component of Lp(a) particles. In particular, the present invention relates to methods for reducing serum Lp(a) levels and reducing the risk of cardiovascular events in patients with elevated levels of Lp(a) comprising administering an LPA-targeted RNAi construct according to specific dosage regimens. Pharmaceutical compositions comprising the LPA-targeted RNAi constructs for use in the methods are also disclosed.

### CROSS-REFERENCE TO RELATED APPLICATIONS

This application claims the benefit of U.S. Provisional Application No. 63/110,309, filed Nov. 5, 2020, which is hereby incorporated by reference in its entirety.

### DESCRIPTION OF THE TEXT FILE SUBMITTED ELECTRONICALLY

The present application contains a Sequence Listing, which has been submitted electronically in ASCII format and is hereby incorporated by reference in its entirety. The computer readable format copy of the Sequence Listing, which was created on Nov. 1, 2021, is named A-2694-WO-PCT ST25 and is 3.5 kilobytes in size.

### FIELD OF THE INVENTION

The present invention relates to pharmaceutical compositions and methods for treating atherosclerotic cardiovascular disease and other conditions associated with elevated lipoprotein (a) (Lp(a)). In particular, the present invention relates to methods for reducing serum levels of Lp(a) and reducing the risk of cardiovascular events, such as cardiovascular death, myocardial infarction, stroke, and coronary revascularization, in patients with elevated Lp(a) by administering an LPA-targeted RNAi construct according to specific dosage regimens.

### BACKGROUND OF THE INVENTION

Atherosclerotic cardiovascular disease is highly prevalent and continues to be the highest cause of mortality worldwide despite the widespread use of low-density lipoprotein (LDL)-lowering therapies. Though LDL-lowering therapies reduce the risk of major cardiac events, the residual cardiovascular risk encountered in some patients with low LDL levels implies other mechanisms of cardiovascular pathology. Over the last decade, compelling evidence from epidemiological studies and meta-analyses, Mendelian randomization studies, and genome wide association studies have shown that an elevated serum Lp(a) concentration is associated with a higher risk of coronary artery disease and atherosclerosis-related disorders (Clarke et al., N. Engl. J. Med., Vol. 361:2518-2528, 2009; Kamstrup et al., JAMA, Vol. 301:2331-2339, 2009; Nordestgaard et al., European Heart Journal, Vol. 31:2844-2853, 2010; Helgadottir et al., J. Am. Coll. Cardiol, Vol. 60:722-729, 2012; Thanassoulis et al., J. Am. Coll. Cardiol, Vol. 55:2491-2498, 2010; Kamstrup et al., J. Am. Coll. Cardiol., Vol. 63:470-477, 2014; Kral et al., Journal of Cardiology, Vol. 118:656-661, 2016; Thanassoulis et al., J. Lipid Res., Vol. 57: 917-924, 2016; Tsimikas et al., J. Am. Coll. Cardiol., Vol. 69:692-711, 2017). In particular, the connection between Lp(a) levels and coronary artery disease, myocardial infarction, stroke, peripheral vascular disease, and aortic valve stenosis has been described in several genetic and observational studies (reviewed in Schmidt et al., J. Lipid Res., Vol. 57:1339-1359, 2016). It has been noted that this risk relationship is continuous and becomes proportionally more impactful with higher Lp(a) levels. The association persists after correction for other lipid parameters (Emerging Risk Factors Collaboration, JAMA, Vol. 302:412-423, 2009).

Lp(a) is a low-density lipoprotein consisting of an LDL particle and the glycoprotein apolipoprotein(a) (apo(a)), which is linked to the apolipoprotein B of the LDL particle by a disulfide bond (Schmidt et al., supra). Apo(a) is encoded by the LPA gene and is expressed almost exclusively in primates, including humans. Apo(a) exhibits homology to plasminogen and is present in various isoforms due to a size polymorphism in the gene, which is caused by a variable number of kringle-IV, type 2 (KIV-2) domain repeats (see Kronenberg and Utermann, J. Intern. Med., Vol. 273:6-30, 2013). An inverse correlation has been observed between the size of the apo(a) isoform and the plasma levels of Lp(a) particles (Sandholzer et al., Hum. Genet., Vol. 86: 607-614, 1991). Lp(a) contains proinflammatory oxidized phospholipids that contribute to its atherogenic effects (Tsimikas et al., J. Am. Coll. Cardiol., Vol. 63:1724-1734, 2014).

High plasma Lp(a) concentration is genetically defined, remains at stable levels, cannot be controlled by habit modifications (diet, exercise, or other environmental factors), and is not effectively controlled by any of the currently available lipid reducing medications. Currently, there are no approved therapies indicated to reduce the risk of cardiovascular events through reductions in Lp(a). Moderate reductions (about 20-30%) in Lp(a) have been observed with proprotein convertase subtilisin/kexin type 9 (PCSK9) inhibitors, niacin, or mipomersen (Santos et al., Arterioscler. Thromb. Vasc. Biol., Vol. 35:689-699, 2015; Yeang et al., Curr. Opin. Lipidol., Vol. 26:169-178, 2015; and Landray et al., N. Engl. J. Med., Vol. 371:203-212, 2014). While apheresis is effective in lowering Lp(a), it is currently used only in a few countries with limited access (Julius, J. Cardiovasc. Dev. Dis., Vol. 5:27-37, 2018). In addition, it is an invasive, very expensive procedure requiring frequent visits, which makes it unfeasible as a long-term treatment for subjects who need lifelong therapy (Khan et al., Eur. Heart J., Vol. 38:1561-1569, 2017; Roeseler et al., Arterioscler. Thromb. Vasc. Biol., Vol. 36:2019-2027, 2016; Leebmann et al., Circulation, Vol. 128:2567-2576, 2013; Safarova et al., Atheroscler. Suppl., Vol. 14:93-99, 2013).

An antisense oligonucleotide targeting the apo(a) messenger RNA transcript (AKCEA-APO(a)-LRx; also known as ISIS 681257 and TQJ230) has been developed and is currently under clinical investigation (reviewed in Graham et al., J Lipid Res., Vol. 57: 340-351, 2016). When administered to healthy subjects having baseline Lp(a) levels of 75 nmol/L or greater at a dose of 10 mg, 20 mg, or 40 mg on days 1, 3, 5, 8, 15, and 22, this molecule was reported to lower Lp(a) concentrations by a mean of 66%, 80%, and 92%, respectively, at day 36 and by a mean of 39%, 53%, and 58%, respectively, at day 113 (Viney et al., Lancet, Vol. 388:2239-2253, 2016). In a subsequent phase 2 study, AKCEA-APO(a)-LRx reduced Lp(a) levels in patients with established cardiovascular disease having baseline Lp(a) levels of 150 nmol/L or greater by a mean of 35%, 56%, and 72% at week 25 when administered to patients at a dose of mg, 40 mg, or 60 mg, respectively, once every 4 weeks (Tsimikas et al., New England Journal of Medicine, Vol. 382:244-255, 2020). A phase 3 cardiovascular outcomes trial in which this molecule is administered at a monthly dose of 80 mg, is ongoing (ClinicalTrials.gov identifier NCT04023552).

However, there remains a need in the art for new therapeutic agents that potently lower Lp(a) concentrations for prolonged durations to enable low dose, low frequency administration regimens for the treatment and prevention of atherosclerotic cardiovascular disease.

### SUMMARY OF THE INVENTION

The present invention is based, in part, on the identification of therapeutic regimens of an LPA-targeted RNAi construct, particularly olpasiran, for effectively reducing circulating Lp(a) levels for the treatment of atherosclerotic cardiovascular disease. Accordingly, in some embodiments, the present invention provides methods for reducing serum or plasma Lp(a) levels in a patient in need thereof comprising administering to the patient an LPA RNAi construct described herein at a dose from about 9 mg to about 675 mg at a dosing interval of at least 8 weeks. In some such embodiments, the patient administered the LPA RNAi construct is diagnosed with or at risk of developing a cardiovascular disease, such as coronary artery disease, carotid artery disease, peripheral artery disease, myocardial infarction, cerebrovascular disease, stroke, aortic valve stenosis, stable or unstable angina, atrial fibrillation, heart failure, hyperlipidemia, heterozygous familial hypercholesterolemia, or homozygous familial hypercholesterolemia. The patient may have a history or a family history of myocardial infarction and/or be diagnosed with acute coronary syndrome. In other embodiments, the patient administered the LPA RNAi construct is diagnosed with chronic kidney disease.

In certain embodiments, the present invention provides methods for treating, reducing or preventing atherosclerosis in a patient in need thereof or treating, reducing, or preventing cardiovascular disease in a patient in need thereof. In such embodiments, the methods comprise administering to the patient an LPA RNAi construct described herein at a dose from about 9 mg to about 675 mg at a dosing interval of at least 8 weeks. The cardiovascular disease to be treated, ameliorated, reduced, or prevented with the methods of the invention can include coronary artery disease, carotid artery disease, peripheral artery disease, myocardial infarction, cerebrovascular disease, stroke, aortic valve stenosis, stable or unstable angina, atrial fibrillation, heart failure, hyperlipidemia, heterozygous familial hypercholesterolemia, or homozygous familial hypercholesterolemia.

The present invention also includes methods for reducing the risk of a cardiovascular event in a patient with atherosclerotic cardiovascular disease. In some embodiments, the methods comprise administering to the patient an LPA RNAi construct described herein at a dose from about 9 mg to about 675 mg at a dosing interval of at least 8 weeks. The cardiovascular event may be a major cardiovascular event, such as cardiovascular death, non-fatal myocardial infarction, non-fatal stroke, or hospitalization for unstable angina. In some embodiments, the cardiovascular event may be a major adverse limb event, such as acute limb ischemia, major amputation, or peripheral revascularization for ischemia. In certain embodiments, the cardiovascular event is cardiovascular death, myocardial infarction, stroke, and/or coronary revascularization. A patient with atherosclerotic cardiovascular disease to be administered the LPA RNAi construct may have a history of coronary revascularization, a history of coronary artery bypass grafting, a diagnosis of coronary artery disease, a diagnosis of atherosclerotic cerebrovascular disease, a diagnosis of peripheral artery disease, and/or a history of myocardial infarction. In one embodiment, the patient to be administered the LPA RNAi construct has experienced a recent myocardial infarction event, e.g. the patient has experienced a myocardial infarction within 1 year prior to the first administration of the LPA RNAi construct. In another embodiment, the patient to be administered the LPA RNAi construct is hospitalized for acute coronary syndrome or unstable angina.

Patients to be administered the LPA RNAi construct according to the methods of the invention have elevated serum or plasma levels of Lp(a). In some embodiments, a patient has a serum or plasma Lp(a) level of about 70 nmol/L or greater prior to the first administration of the LPA RNAi construct. In other embodiments, a patient has a serum or plasma Lp(a) level of about 150 nmol/L or greater prior to the first administration of the LPA RNAi construct. In certain embodiments, a patient has a serum or plasma Lp(a) level of about 175 nmol/L or greater prior to the first administration of the LPA RNAi construct. In certain other embodiments, a patient has a serum or plasma Lp(a) level of about 200 nmol/L or greater prior to the first administration of the LPA RNAi construct.

In some embodiments of the methods of the invention, a patient to be administered the LPA RNAi construct is receiving a lipid-lowering therapy, for example to reduce a patient's LDL-C levels. The lipid-lowering therapy may be a PCSK9 inhibitor, such as a PCSK9 antagonist monoclonal antibody (e.g. evolocumab, alirocumab), a statin (e.g. atorvastatin, cerivastatin, fluvastatin, lovastatin, mevastatin, pitavastatin, pravastatin, rosuvastatin, simvastatin), a cholesterol absorption inhibitor (e.g. ezetimibe), bempedoic acid, nicotinic acid (e.g. niacin), fibric acid (e.g. gemfibrozil, fenofibrate), a bile acid sequestrant (e.g. cholestyramine, colestipol, colesevelam), LDL apheresis, or combinations thereof. In these and other embodiments, the patient may have a serum LDL-C level of about 100 mg/dL or less or about 70 mg/dL or less prior to the first administration of the LPA RNAi construct.

In certain embodiments of the methods of the invention, a fixed dose of the LPA RNAi construct is administered to a patient once every 12 weeks or once every 3 months. In some such embodiments, the fixed dose may be from about 10 mg to about 225 mg, from about 75 mg to about 225 mg, from about 50 mg to about 100 mg, or from about 150 mg to about 225 mg. In one embodiment, the LPA RNAi construct is administered to the patient at a fixed dose of about mg once every 12 weeks or once every 3 months. In another embodiment, the LPA RNAi construct is administered to the patient at a fixed dose of about 75 mg once every 12 weeks or once every 3 months. In yet another embodiment, the LPA RNAi construct is administered to the patient at a fixed dose of about 150 mg once every 12 weeks or once every 3 months. In still another embodiment, the LPA RNAi construct is administered to the patient at a fixed dose of about 225 mg once every 12 weeks or once every 3 months.

In certain other embodiments of the methods of the invention, a fixed dose of the LPA RNAi construct is administered to a patient once every 24 weeks or once every 6 months. In some such embodiments, the fixed dose may be from about 225 mg to about 675 mg, from about 225 mg to about 450 mg, or from about 200 mg to about 300 mg. In some embodiments, the LPA RNAi construct is administered to the patient at a fixed dose of about 225 mg once every 24 weeks or once every 6 months. In other embodiments, the LPA RNAi construct is administered to the patient at a fixed dose of about 300 mg once every 24 weeks or once every 6 months. In certain embodiments, the LPA RNAi construct is administered to the patient at a fixed dose of about 450 mg once every 24 weeks or once every 6 months. In certain other embodiments, the LPA RNAi construct is administered to the patient at a fixed dose of about 675 mg once every 24 weeks or once every 6 months.

Administration of an LPA RNAi construct to a patient according to the methods of the invention substantially reduces a patient's plasma or serum Lp(a) for prolonged periods of time. For instance, in some embodiments of the methods of the invention, administration of the LPA RNAi construct reduces serum or plasma Lp(a) levels in the patient by greater than 80% for at least 12 weeks, at least 16 weeks, or at least 24 weeks as compared to the patient's baseline serum or plasma Lp(a) levels. In other embodiments of the methods of the invention, administration of the LPA RNAi construct reduces serum or plasma Lp(a) levels in the patient by greater than 90% for at least 12 weeks, at least 16 weeks, or at least 24 weeks as compared to the patient's baseline serum or plasma Lp(a) levels. In certain embodiments, administration of an LPA RNAi construct to a patient according to the methods of the invention reduces a patient's plasma or serum Lp(a) level to about 100 nmol/L or less. In some embodiments, administration of an LPA RNAi construct to a patient according to the methods of the invention reduces a patient's plasma or serum Lp(a) level to about 75 nmol/L or less. In other embodiments, administration of an LPA RNAi construct to a patient according to the methods of the invention reduces a patient's plasma or serum Lp(a) level to about 50 nmol/L or less.

In any embodiments of the methods disclosed herein, the LPA RNAi construct administered to a patient can be a double-stranded RNA molecule, such as an siRNA molecule, comprising a sense strand and an antisense strand, wherein the antisense strand comprises a region having a sequence that is complementary to an LPA mRNA sequence. Preferably, the sense strand comprises a sequence that is sufficiently complementary to the sequence of the antisense strand to form a duplex region of about 15 to about 30 base pairs in length. In certain embodiments of the methods of the invention, the LPA RNAi construct administered to a patient comprises a sense strand and an antisense strand, each of which is about 19 to about 23 nucleotides in length, wherein the antisense strand comprises a sequence that is complementary to an LPA mRNA sequence and the sense strand comprises a sequence that is complementary to the sequence of the antisense strand. In one such embodiment, the sense strand and antisense strand of the LPA RNAi construct can each be 21 nucleotides in length and hybridize to each other to form a duplex region that is 21 base pairs in length such that the RNAi construct has two blunt ends. In another such embodiment, the sense strand and antisense strand of the LPA RNAi construct can each be 19 nucleotides in length and hybridize to each other to form a duplex region that is 19 base pairs in length such that the RNAi construct has two blunt ends.

In some embodiments of the methods of the invention, the LPA RNAi construct administered to a patient further comprises a targeting moiety comprising an asialoglycoprotein receptor ligand, wherein the targeting moiety is covalently attached to the sense strand, for example, to the 5′ end of the sense strand. The targeting moiety can comprise a trivalent GalNAc moiety, such as the moiety having the structure of Structure 1 described herein. In certain embodiments, the LPA RNAi construct administered to a patient according to the methods of the invention comprises a sense strand comprising the sequence of SEQ ID NO: 1 and an antisense strand comprising the sequence of SEQ ID NO: 2. In some embodiments, the LPA RNAi construct comprises a sense strand comprising or consisting of the sequence of SEQ ID NO: 3 and an antisense strand comprising or consisting of the sequence of SEQ ID NO: 4. In certain preferred embodiments, the sense strand and/or antisense strand of the LPA RNAi construct comprises one or more modified nucleotides. In such embodiments, the LPA RNAi construct comprises a sense strand comprising or consisting of the sequence of modified nucleotides according to SEQ ID NO: 5 and an antisense strand comprising or consisting of the sequence of modified nucleotides according to SEQ ID NO: 6. In preferred embodiments, the LPA RNAi construct administered to a patient according to the methods of the invention is olpasiran.

The present invention also provides pharmaceutical compositions comprising an LPA RNAi construct, such as olpasiran, for use in the methods of the invention described herein. The pharmaceutical compositions can comprise one or more pharmaceutically acceptable diluents, carriers, or excipients. In certain embodiments, the pharmaceutical compositions comprise an LPA RNAi construct (e.g. olpasiran), a potassium phosphate buffer, and sodium chloride, wherein the composition has a pH of about 6.6 to about 7.0, preferably about 6.8. Any of the pharmaceutical compositions described herein can be incorporated into injection devices, such as pre-filled syringes, autoinjectors, injection pumps, on-body injectors, and injection pens, for administration (e.g. subcutaneous administration) to a patient according to the methods described herein. In some embodiments, administration of the LPA RNAi construct (e.g. olpasiran) or pharmaceutical composition comprising the LPA RNAi construct (e.g. olpasiran) to a patient according to the methods of the invention is by subcutaneous injection. In such embodiments, the injection volume is about 2 mL or less or about 1 mL or less, for example about 1 mL.

The use of LPA RNAi constructs in any of the methods disclosed herein or for preparation of medicaments for administration according to any of the methods disclosed herein is specifically contemplated. For instance, the present invention includes an LPA RNAi construct for use in a method for treating, reducing, or preventing atherosclerosis or cardiovascular disease in a patient in need thereof, wherein the method comprises administering to the patient the LPA RNAi construct at a dose from about 9 mg to about 675 mg at a dosing interval of at least 8 weeks. The present invention also includes an LPA RNAi construct for use in a method for reducing serum or plasma Lp(a) levels in a patient, wherein the method comprises administering to the patient the LPA RNAi construct at a dose from about 9 mg to about 675 mg at a dosing interval of at least 8 weeks. In certain embodiments, the present invention provides an LPA RNAi construct for use in a method for reducing the risk of a cardiovascular event in a patient with atherosclerotic cardiovascular disease, wherein the method comprises administering to the patient the LPA RNAi construct at a dose from about 9 mg to about 675 mg at a dosing interval of at least 8 weeks.

The present invention also encompasses the use of an LPA RNAi construct in the preparation of a medicament for treating, reducing, or preventing atherosclerosis or cardiovascular disease in a patient in need thereof, wherein the medicament is administered or formulated for administration at a dose from about 9 mg to about 675 mg at a dosing interval of at least 8 weeks. In some embodiments, the present invention provides the use of an LPA RNAi construct in the preparation of a medicament for reducing serum or plasma Lp(a) levels in a patient, wherein the medicament is administered or formulated for administration at a dose from about 9 mg to about 675 mg at a dosing interval of at least 8 weeks. In other embodiments, the present invention provides the use of an LPA RNAi construct in the preparation of a medicament for reducing the risk of a cardiovascular event in a patient with atherosclerotic cardiovascular disease, wherein the medicament is administered or formulated for administration at a dose from about 9 mg to about 675 mg at a dosing interval of at least 8 weeks.

### BRIEF DESCRIPTION OF THE DRAWINGS

FIG. 1 depicts the structure of the LPA RNAi construct, olpasiran, schematically. The top strand listed in the 5′ to 3′ direction is the sense strand (SEQ ID NO: 5) and the bottom strand listed in the 3′ to 5′ direction is the antisense strand (SEQ ID NO: 6). Black circles represent nucleotides with a 2′-O-methyl modification, white circles represent nucleotides with a 2′-deoxy-2′-fluoro modification, and the gray circle represents a deoxyadenosine nucleotide linked to the adjacent nucleotide via a 3′-3′ linkage (i.e. inverted). Gray lines connecting the circles represent phosphodiester linkages, whereas the black lines connecting the circles represent phosphorothioate linkages. A trivalent GalNAc moiety having the depicted structure is represented by R1 and is covalently attached to the 5′ end of the sense strand by a phosphorothioate linkage.

FIG. 2 is a line graph showing the percent change from baseline in plasma Lp(a) levels in human subjects after a single subcutaneous dose of placebo or olpasiran at the indicated doses in each of cohorts 1 to 7 over study days. Baseline values were the mean of screening Lp(a) and day 1 pre-dose Lp(a) levels. If only 1 value was available, that value was used as the baseline value.

FIGS. 3A-3F show the predicted Lp(a) levels as a percentage of baseline for quarterly (Q3M) dosing of olpasiran at doses of 10 mg (FIG. 3A), 30 mg (FIG. 3B), 50 mg (FIG. 3C), 75 mg (FIG. 3D), 150 mg (FIG. 3E), and 225 mg (FIG. 3F) for subjects with baseline Lp(a) levels of ≥150 nmol/L. The horizontal line in each of the graphs represents 80% reduction of Lp(a) levels from baseline. The predicted Lp(a) levels are based on PK/PD model simulations for 10,000 subjects. Predicted data are shown as median values (solid line) with 95% prediction interval represented by shading. The solid circles in FIG. 3D represent observed data from cohort 7 described in Example 1.

FIGS. 4A-4F show the predicted Lp(a) levels as a percentage of baseline for biannual (Q6M) dosing of olpasiran at doses of 10 mg (FIG. 4A), 75 mg (FIG. 4B), 150 mg (FIG. 4C), 225 mg (FIG. 4D), 450 mg (FIG. 4E), and 675 mg (FIG. 4F) for subjects with baseline Lp(a) levels of ≥150 nmol/L. The horizontal line in each of the graphs represents 80% reduction of Lp(a) levels from baseline. The predicted Lp(a) levels are based on PK/PD model simulations for 10,000 subjects. Predicted data are shown as median values (solid line) with 95% prediction interval represented by shading. The solid circles in FIG. 4B represent observed data from cohort 7 described in Example 1.

### DETAILED DESCRIPTION

Lp(a) has been reported to be a causal risk factor for various forms of cardiovascular disease, including myocardial infarction, stroke, peripheral artery disease, and aortic stenosis. Lp(a) concentrations are genetically determined and unlike LDL cholesterol (LDL-C) concentrations, cannot be modified by diet, exercise, or other lifestyle changes. Currently, there are no approved therapies that selectively target apo(a) and substantially reduce Lp(a) levels. The present invention provides novel dosage regimens of an RNAi construct targeting a mRNA transcribed from the LPA gene, which encodes the apo(a) protein, for sustained suppression of Lp(a) levels for treatment or prevention of atherosclerosis and related cardiovascular conditions. A particular LPA-targeted RNAi construct, olpasiran, was observed to reduce Lp(a) concentrations in human subjects with baseline Lp(a) levels of ≥70 nmol/L by 71% to 96% after single doses, with maximal percent reductions of >90% and effects persisting for more than 6 months at single doses of 9 mg or higher (see Example 1). Specifically, single doses as low as 9 mg of olpasiran reduced Lp(a) levels in human subjects by greater than 80% for greater than 3 months, whereas single olpasiran doses of 75 mg and 225 mg suppressed Lp(a) levels by greater than 80% for more than six months. Olpasiran was also well-tolerated at these doses and there were no treatment-related serious adverse events (see Example 1). The robust and sustained suppression of Lp(a) in this dosage range was unexpected as 8-fold higher doses (e.g. 75 mg vs. 9 mg) were predicted to be required to produce an 80% reduction in Lp(a) for one month based on allometric scaling of olpasiran doses evaluated in cynomolgus monkeys.

The depth and duration of Lp(a) suppression in human subjects with olpasiran were also surprising in view of results reported in human subjects with other nucleic acid therapeutics targeting apo(a). AKCEA-APO(a)-LRx, an antisense oligonucleotide targeting apo(a), has been reported to reduce Lp(a) levels in human subjects from 35% to 80% after six months of treatment. However, weekly doses of 20 mg or monthly doses of 60 mg of AKCEA-APO(a)-LRx were required to achieve an 80% reduction and a 72% reduction, respectively, in Lp(a) levels (see Tsimikas et al., New England Journal of Medicine, Vol. 382:244-255, 2020). In contrast, as described herein, single doses of olpasiran produced reductions of greater than 80% in Lp(a) levels for longer than six months, thereby allowing for administration of olpasiran at lower doses and longer dosing intervals, such as once every 3 months or once every 6 months. Thus, the methods of the present invention provide significant improvements in treating humans for atherosclerotic cardiovascular disease, including, for example, improved patient adherence, reduced cost of medication, and reduced volume and number of injections. Accordingly, in certain embodiments, the present invention provides methods for treating, preventing, or reducing the risk of developing a cardiovascular disease in a patient in need thereof comprising administering to the patient an effective amount of an LPA RNAi construct according to specific dosage regimens as described herein.

Atherosclerosis is a disease in which plaques made up of fatty substances, cholesterol, calcium, fibrin, and cellular waste products build up in various arteries in the body. Over time, the plaques harden and narrow the lumen of the arteries, thereby restricting blood flow to organs and tissues in the body. Atherosclerosis can lead to the development of a number of other diseases, such as cardiovascular disease, cerebrovascular disease, or chronic kidney disease, based on the specific arteries which are affected by atherosclerotic plaque accumulation. For example, coronary artery disease occurs when plaques build up in the coronary arteries and partially block the flow of blood to the heart, which can lead to angina and a myocardial infarction. Atherosclerotic plaque build-up in the carotid arteries, which supply oxygen-rich blood to the brain, results in carotid artery disease and can cause a transient ischemic attack or stroke if the blood flow is reduced or blocked. Peripheral artery disease occurs when plaques build up in the major arteries supplying blood to the limbs and pelvis and can lead to abdominal aortic aneurysms and limb ischemia causing numbness and pain. When atherosclerotic plaques accumulate in the renal arteries, chronic kidney disease develops and can lead to decreased kidney function over time that can result in kidney failure. Lp(a) is an atherogenic lipoprotein, elevated levels of which have been associated with increased risk of coronary artery disease, peripheral artery disease, myocardial infarction, and stroke, in particular. The methods of the invention are useful for treating, reducing, or preventing atherosclerosis in a patient by reducing circulating Lp(a) levels. Thus, in some embodiments, the present invention provides methods for treating, reducing, or preventing atherosclerosis in a patient in need thereof comprising administering to the patient an effective amount of an LPA RNAi construct according to any of the dosage regimens as described herein. In one embodiment, the present invention includes use of any of the LPA RNAi constructs described herein for preparation of a medicament for treating, reducing, or preventing atherosclerosis in a patient in need thereof, wherein the medicament is administered or formulated for administration according to any of the dosage regimens described herein. In another embodiment, the present invention provides an LPA RNAi construct, such as any of the LPA RNAi constructs described herein, for use in a method for treating, reducing, or preventing atherosclerosis in a patient in need thereof, wherein the method comprises administering the LPA RNAi construct according to any of the dosage regimens described herein.

In certain embodiments, the present invention also provides methods for treating, reducing, ameliorating, or preventing a cardiovascular disease in a patient in need thereof comprising administering to the patient an effective amount of an LPA RNAi construct according to any of the dosage regimens as described herein. In some embodiments, the present invention includes use of any of the LPA RNAi constructs described herein for preparation of a medicament for treating, reducing, or preventing a cardiovascular disease in a patient in need thereof, wherein the medicament is administered or formulated for administration according to any of the dosage regimens described herein. In other embodiments, the present invention provides an LPA RNAi construct, such as any of the LPA RNAi constructs described herein, for use in a method for treating, reducing, or preventing a cardiovascular disease in a patient in need thereof, wherein the method comprises administering the LPA RNAi construct according to any of the dosage regimens described herein. Cardiovascular disease is a class of diseases and conditions that affect the blood vessels or heart and includes, but is not limited to, myocardial infarction, heart failure, transient ischemic attack, stroke (ischemic and hemorrhagic), atherosclerosis, coronary artery disease, peripheral vascular disease (e.g. peripheral artery disease), aneurysm (e.g. abdominal aortic aneurysm), carotid artery disease, cerebrovascular disease, stable or unstable angina, atrial fibrillation, hyperlipidemia, familial hypercholesterolemia (heterozygous and homozygous), vulnerable plaque, and aortic valve stenosis. Thus, in certain embodiments, the patients to be treated according to the methods of the invention are diagnosed with or at risk of developing cardiovascular disease. A patient who is at risk of developing cardiovascular disease may have a family history of cardiovascular disease and/or may have one or more risk factors for cardiovascular disease. Such risk factors include, but are not limited to, hypertension, elevated levels of non-HDL cholesterol, elevated levels of triglycerides, diabetes, obesity, or tobacco use. Diagnosis of atherosclerosis and cardiovascular disease can be made using a variety of methods known to those of skill in the art and may include one or more of the following: patient medical and family history, risk factors of the patient, physical examination, blood tests to measure various biomarkers, such as lipid levels (e.g. LDL-C, triglycerides, Lp(a), glycated hemoglobin A1C, C-reactive protein, apolipoprotein B, cardiac troponin-T, etc.), electrocardiogram, echocardiogram, stress testing, chest X-ray, computed tomography (CT) scan (e.g. cardiac CT scan), and angiography.

In some embodiments, the cardiovascular disease to be treated, reduced, ameliorated, or prevented according to the methods of the invention is coronary artery disease. Signs and symptoms of coronary artery disease may include chest pain (e.g. angina), shortness of breath, myocardial infarction, stenosis of one or more coronary arteries, pain or discomfort in arms or shoulders, weakness, dizziness, nausea, and history of coronary artery bypass and/or percutaneous coronary artery intervention. In related embodiments, the cardiovascular disease to be treated, reduced, ameliorated, or prevented according to the methods of the invention is myocardial infarction.

In other embodiments, the cardiovascular disease to be treated, reduced, ameliorated, or prevented according to the methods of the invention is cerebrovascular disease, particularly atherosclerotic cerebrovascular disease. Cerebrovascular disease refers to disorders in which an area of the brain is temporarily or permanently affected by ischemia or bleeding due to dysfunction or complications with one or more of the cerebral blood vessels. Cerebrovascular diseases include, but are not limited to, transient ischemic attack, stroke (ischemic or hemorrhagic), carotid artery stenosis, vertebral artery stenosis, intracranial artery stenosis, aneurysms, and vascular malformations. Signs and symptoms of cerebrovascular disease may include dizziness, nausea, vomiting, unusually severe headache, confusion, disorientation, memory loss, numbness or weakness in an arm, leg or the face, especially on one side, abnormal or slurred speech, difficulty with comprehension, loss of vision or difficulty seeing, loss of balance, coordination or the ability to walk, carotid artery stenosis, and history of transient ischemic attacks and/or carotid artery revascularizations. In one embodiment, the cardiovascular disease to be treated, reduced, ameliorated, or prevented according to the methods of the invention is stroke.

In certain other embodiments, the cardiovascular disease to be treated or prevented according to the methods of the invention is peripheral artery disease. Signs and symptoms of peripheral artery disease can include pain or muscle cramps in the legs or arms while walking (claudication), leg numbness or weakness, coldness in lower leg or foot, sores on toes, feet or legs that do not heal, change in the color of the legs, hair loss or slower hair growth on the feet and legs, slower growth of toenails, shiny skin on legs, no pulse or a weak pulse in the legs or feet, ankle brachial index≤0.90, and history of abdominal aortic aneurysm, abdominal aorta treatment (percutaneous or surgical), and/or peripheral artery revascularization (percutaneous or surgical).

In some embodiments, administration of the LPA RNAi constructs according to the methods of the invention is for the treatment of atherosclerosis and other cardiovascular diseases and conditions. The term “treatment” or “treat” as used herein refers to the application or administration of the LPA RNAi construct to a patient who has or is diagnosed with atherosclerosis or other cardiovascular disease, has a symptom of atherosclerosis or other cardiovascular disease, is at risk of developing atherosclerosis or other cardiovascular disease, or has a predisposition to atherosclerosis or other cardiovascular disease for the purpose of curing, healing, alleviating, relieving, altering, ameliorating, or improving atherosclerosis or other cardiovascular disease, one or more symptoms of atherosclerosis or other cardiovascular disease, the risk of developing atherosclerosis or other cardiovascular disease, or predisposition toward atherosclerosis or other cardiovascular disease. The term “treatment” encompasses any improvement of the disease in the patient, including the slowing or stopping of the progression of atherosclerosis or other cardiovascular disease in the patient, a decrease in the number or severity of the symptoms of atherosclerosis or other cardiovascular disease, or an increase in frequency or duration of periods where the patient is free from the symptoms of atherosclerosis or other cardiovascular disease. The term “patient,” as used herein, refers to a mammal, including humans, and can be used interchangeably with the term “subject.” In preferred embodiments, the patient is a human patient.

In certain preferred embodiments, administration of the LPA RNAi construct to a patient according to any of the methods of the invention reduces circulating Lp(a) levels or concentrations (e.g. serum or plasma Lp(a) levels/concentrations) in the patient as compared to the circulating Lp(a) levels in the patient prior to administration of the LPA RNAi construct (e.g. a baseline Lp(a) level/concentration) or as compared to the circulating Lp(a) level/concentration in a patient not receiving the LPA RNAi construct. Accordingly, in some embodiments, the present invention provides a method for reducing serum or plasma Lp(a) levels (or concentrations) in a patient in need thereof comprising administering to the patient an LPA RNAi construct according to any of the dosage regimens as described herein. In one embodiment, the present invention includes use of any of the LPA RNAi constructs described herein for the preparation of a medicament for reducing serum or plasma Lp(a) levels (or concentrations) in a patient in need thereof, wherein the medicament is administered or formulated for administration according to any of the dosage regimens described herein. In another embodiment, the present invention provides an LPA RNAi construct, such as any of the LPA RNAi constructs described herein, for use in a method for reducing serum or plasma Lp(a) levels (or concentrations) in a patient in need thereof, wherein the method comprises administering the LPA RNAi construct according to any of the dosage regimens described herein. In some embodiments, a patient in need of reduction of serum or plasma Lp(a) levels (or concentrations) is a patient diagnosed with or at risk of cardiovascular disease, such as any of the cardiovascular diseases described above. In some such embodiments, the cardiovascular disease is coronary artery disease, carotid artery disease, peripheral artery disease, myocardial infarction, cerebrovascular disease, stroke, aortic valve stenosis, stable or unstable angina, atrial fibrillation, heart failure, hyperlipidemia, heterozygous familial hypercholesterolemia, or homozygous familial hypercholesterolemia. In one particular embodiment, a patient in need of reduction of serum or plasma Lp(a) levels (or concentrations) has a history of myocardial infarction or has a family history of myocardial infarction.

In certain embodiments, a patient in need of reduction of serum or plasma Lp(a) levels (or concentrations) is diagnosed with acute coronary syndrome. Acute coronary syndrome refers to conditions associated with a sudden reduction of blood flow to the heart, often caused by a rupture of an atherosclerotic plaque and partial or complete thrombosis of a coronary artery. Acute coronary syndromes include an acute myocardial ischemia or infarction, such as non-ST-elevation myocardial infarction (NSTEMI) and ST-elevation MI (STEMI), as well as unstable angina. Even if acute coronary syndrome does not result in an infarct initially, it is a sign of a high risk of an infarct occurring and must be promptly diagnosed and treated. Signs and symptoms of acute coronary syndrome typically begin abruptly and can include chest pain (angina) or discomfort, pain spreading from the chest to the shoulders, arms, upper abdomen, back, neck or jaw, nausea or vomiting, indigestion, shortness of breath, sudden heavy sweating, lightheadedness, dizziness or fainting, unusual or unexplained fatigue, and feelings of restless or apprehension.

In certain other embodiments, a patient in need of reduction of serum or plasma Lp(a) levels (or concentrations) is diagnosed with chronic kidney disease. Chronic kidney disease generally refers to gradual damage to the kidneys and loss of function. As chronic kidney disease worsens over time, a patient can be at increased risk for other cardiovascular diseases. In one embodiment, a patient to be treated according to the methods of the invention has stage 3 chronic kidney disease. The stages of kidney disease are determined by estimated glomerular filtration rate (eGFR), which is a value based on the amount of creatinine in the blood. Stage 3 chronic kidney disease is characterized by an eGFR of about 30 mL/min/1.73 m² to about 59 mL/min/1.73 m² and may be accompanied by some initial symptoms, such as swelling in the hands and feet, back pain, and urinating more or less than normal. Patients with stage 3 chronic kidney disease may also have other health-related issues, such as hypertension, anemia, and bone disease. In another embodiment, a patient to be treated according to the methods of the invention has stage 4 chronic kidney disease. A patient with stage 4 chronic kidney disease has an eGFR of about 15 mL/min/1.73 m 2 to about 29 mL/min/1.73 m 2 and will typically exhibit symptoms like swelling in the hands and feet, back pain, and urinating more or less than normal.

In some embodiments, administration of the LPA RNAi construct to a patient according to the methods of the invention reduces Lp(a) levels (or concentrations) in serum or plasma in the patient by at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, or at least about 95% as compared to the Lp(a) levels (or concentrations) in serum or plasma in the patient prior to administration of the RNAi construct (e.g. baseline Lp(a) level or concentration) or as compared to the Lp(a) levels (or concentrations) in serum or plasma in a patient not receiving the RNAi construct. In these and other embodiments, following administration of the LPA RNAi construct (e.g. administration of a single dose of the LPA RNAi construct), circulating Lp(a) levels or concentrations are reduced in the patient for at least 4 weeks, at least 6 weeks, at least 8 weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, at least 30 weeks, at least 32 weeks, at least 36 weeks, or least 48 weeks.

In one embodiment of the methods of the invention, administration of the LPA RNAi construct (e.g. a single dose of the LPA RNAi construct) reduces serum or plasma Lp(a) levels (or concentrations) in the patient by greater than 50% for at least 12 weeks as compared to the patient's baseline serum or plasma Lp(a) levels (or concentrations). Baseline serum or plasma Lp(a) levels (or concentrations) refers to the serum or plasma Lp(a) levels (or concentrations) in a patient prior to administration of the LPA RNAi construct (i.e. pre-treatment levels or concentrations). A baseline level/concentration may be a single measurement taken prior to the patient receiving the LPA RNAi construct or a baseline level/concentration may be an average of two or more measurements taken prior to the patient receiving the LPA RNAi construct. In another embodiment of the methods of the invention, administration of the LPA RNAi construct (e.g. a single dose of the LPA RNAi construct) reduces serum or plasma Lp(a) levels (or concentrations) in the patient by greater than 50% for at least 24 weeks as compared to the patient's baseline serum or plasma Lp(a) levels (or concentrations). In yet another embodiment of the methods of the invention, administration of the LPA RNAi construct (e.g. a single dose of the LPA RNAi construct) reduces serum or plasma Lp(a) levels (or concentrations) in the patient by greater than 80% for at least 12 weeks as compared to the patient's baseline serum or plasma Lp(a) levels (or concentrations). In still another embodiment of the methods of the invention, administration of the LPA RNAi construct (e.g. a single dose of the LPA RNAi construct) reduces serum or plasma Lp(a) levels (or concentrations) in the patient by greater than 80% for at least 24 weeks as compared to the patient's baseline serum or plasma Lp(a) levels (or concentrations). In certain embodiments of the methods of the invention, administration of the LPA RNAi construct (e.g. a single dose of the LPA RNAi construct) reduces serum or plasma Lp(a) levels (or concentrations) in the patient by greater than 80% for at least 32 weeks as compared to the patient's baseline serum or plasma Lp(a) levels (or concentrations). In some embodiments of the methods of the invention, administration of the LPA RNAi construct (e.g. a single dose of the LPA RNAi construct) reduces serum or plasma Lp(a) levels (or concentrations) in the patient by greater than 90% for at least 12 weeks as compared to the patient's baseline serum or plasma Lp(a) levels (or concentrations). In other embodiments of the methods of the invention, administration of the LPA RNAi construct (e.g. a single dose of the LPA RNAi construct) reduces serum or plasma Lp(a) levels (or concentrations) in the patient by greater than 90% for at least 16 weeks as compared to the patient's baseline serum or plasma Lp(a) levels (or concentrations).

In certain embodiments, administration of an LPA RNAi construct to a patient according to the methods of the invention reduces absolute Lp(a) levels (or concentrations) in serum or plasma in the patient to about 150 nmol/L or less, about 125 nmol/L or less, about 100 nmol/L or less, about 75 nmol/L or less, about 70 nmol/L or less, about 65 nmol/L or less, about 60 nmol/L or less, about 55 nmol/L or less, about 50 nmol/L, about 45 nmol/L or less, about 40 nmol/L or less, about 35 nmol/L or less, or about 30 nmol/L or less. In one embodiment, administration of an LPA RNAi construct to a patient according to the methods of the invention reduces absolute Lp(a) levels (or concentrations) in serum or plasma in the patient to about 125 nmol/L or less. In another embodiment, administration of an LPA RNAi construct to a patient according to the methods of the invention reduces absolute Lp(a) levels (or concentrations) in serum or plasma in the patient to about 100 nmol/L or less. In another embodiment, administration of an LPA RNAi construct to a patient according to the methods of the invention reduces absolute Lp(a) levels (or concentrations) in serum or plasma in the patient to about 75 nmol/L or less. In yet another embodiment, administration of an LPA RNAi construct to a patient according to the methods of the invention reduces absolute Lp(a) levels (or concentrations) in serum or plasma in the patient to about 50 nmol/L or less.

Although there is a preference to measure Lp(a) levels/concentrations in units of particle concentration (e.g. nmol/L)(see, e.g., Wilson et al., Journal of Clinical Lipidology, Vol. 13: 374-392, 2019), Lp(a) levels may be measured in units of mass concentration (e.g. mg/dL). In such embodiments, administration of an LPA RNAi construct to a patient according to the methods of the invention may reduce Lp(a) levels (or concentrations) in serum or plasma in the patient to about 100 mg/dL or less, about 90 mg/dL or less, about 80 mg/dL or less, about 70 mg/dL or less, about 60 mg/dL or less, about 50 mg/dL or less, about 45 mg/dL or less, about 40 mg/dL or less, about 35 mg/dL or less, about 30 mg/dL or less, about 25 mg/dL or less, about 20 mg/dL or less, or about 15 mg/dL or less.

Lp(a) levels can be measured in plasma or serum samples using commercially available kits, such as the Lp(a) ELISA assay kit from Mercodia AB (Uppsala, Sweden), the Lp(a) immunoturbidimetric assay from Randox Laboratories Ltd. (Crumlin, United Kingdom), or the Tina-quant® Lp(a) Gen. 2 assay from F. Hoffmann-La Roche Ltd. (Basel, Switzerland), or using other methods known in the art, such as those described Marcovina and Albers, J. Lipid Res., Vol. 57:526-537, 2016. In certain embodiments, Lp(a) levels are measured using a turbidimetric immunoassay that is standardized to detect and quantitate Lp(a) particles independent of apo(a) isoform size. In these and other embodiments, the assay used to measure Lp(a) levels is standardized against the IFCC reference material SRM2B for nmol/L (Marcovina et al., Clin. Chem., Vol. 46: 1946-1967, 2000).

As described above, elevated levels of circulating Lp(a) are associated with an increased risk of cardiovascular disease. Thus, the methods of the invention are also useful for reducing the risk of cardiovascular events in patients who have elevated serum or plasma levels of Lp(a). Accordingly, in certain embodiments, the present invention provides methods for reducing the risk of a cardiovascular event in a patient with atherosclerotic cardiovascular disease comprising administering to the patient an effective amount of an LPA RNAi construct according to any of the dosage regimens as described herein. In one embodiment, the present invention includes use of any of the LPA RNAi constructs described herein for preparation of a medicament for reducing the risk of a cardiovascular event in a patient with atherosclerotic cardiovascular disease, wherein the medicament is administered or formulated for administration according to any of the dosage regimens described herein. In another embodiment, the present invention provides an LPA RNAi construct, such as any of the LPA RNAi constructs described herein, for use in a method for reducing the risk of a cardiovascular event in a patient with atherosclerotic cardiovascular disease, wherein the method comprises administering the LPA RNAi construct according to any of the dosage regimens described herein.

In some embodiments, the cardiovascular event is one or more of the following: cardiovascular death, myocardial infarction, stroke (e.g. ischemic stroke), coronary revascularization, hospitalization for unstable angina, hospitalization for heart failure, peripheral revascularization, acute limb ischemia, transient ischemic attack, major limb amputation for ischemia, cerebrovascular revascularization, and all cause death. In certain embodiments, the cardiovascular event is cardiovascular death, myocardial infarction, stroke (e.g. ischemic stroke), and/or coronary revascularization. In some such embodiments, the cardiovascular event is cardiovascular death, myocardial infarction, and/or coronary revascularization. In other such embodiments, the cardiovascular event is myocardial infarction and/or coronary revascularization. In other embodiments, the cardiovascular event is a major cardiovascular event selected from cardiovascular death, non-fatal myocardial infarction, non-fatal stroke, and hospitalization for unstable angina. In still other embodiments, the cardiovascular event is a major adverse limb event selected from acute limb ischemia, major amputation, and peripheral revascularization for ischemia. In one embodiment, the cardiovascular event is cardiovascular death. In another embodiment, the cardiovascular event is non-fatal myocardial infarction. In yet another embodiment, the cardiovascular event is non-fatal stroke (e.g. ischemic stroke). In still another embodiment, the cardiovascular event is coronary revascularization.

In certain embodiments, a patient administered an LPA RNAi construct according to the methods of the invention has a relative risk reduction of at least 15%, at least 20%, at least 25%, or at least 30% for any of the cardiovascular events described above as compared to a patient not receiving the LPA RNAi construct. In one embodiment, a patient administered an LPA RNAi construct according to the methods of the invention has a relative risk reduction of about 15% to about 25% for any one of cardiovascular death, myocardial infarction, and ischemic stroke as compared to a patient not receiving the LPA RNAi construct. In another embodiment, a patient administered an LPA RNAi construct according to the methods of the invention has a relative risk reduction of about 20% to about 30% for any one of cardiovascular death, myocardial infarction, and ischemic stroke as compared to a patient not receiving the LPA RNAi construct.

In certain other embodiments, a patient administered an LPA RNAi construct according to the methods of the invention has an absolute risk reduction of at least 1.5%, at least 1.8%, at least 2.0%, at least 2.2%, at least 2.5%, at least 2.8%, at least 3.0%, at least 3.2%, or at least 3.5% for any of the cardiovascular events described above. In one embodiment, a patient administered an LPA RNAi construct according to the methods of the invention has an absolute risk reduction of about 1.5% to about 3.0% for any one of cardiovascular death, myocardial infarction, and ischemic stroke. In another embodiment, a patient administered an LPA RNAi construct according to the methods of the invention has an absolute risk reduction of about 2.0% to about 3.5% for any one of cardiovascular death, myocardial infarction, and ischemic stroke. In yet another embodiment, a patient administered an LPA RNAi construct according to the methods of the invention has an absolute risk reduction of about 2.0% to about 3.0% for any one of cardiovascular death, myocardial infarction, and ischemic stroke.

In any of the above-described embodiments, a patient administered an LPA RNAi construct according to the methods of the invention for reducing a cardiovascular event may have a history of coronary revascularization, a history of coronary artery bypass grafting, a diagnosis of coronary artery disease, a diagnosis of atherosclerotic cerebrovascular disease, a diagnosis of peripheral artery disease, and/or a history of myocardial infarction. In certain embodiments, a patient administered an LPA RNAi construct according to the methods of the invention for reducing a cardiovascular event has experienced a myocardial infarction. For instance, in some such embodiments, a patient administered an LPA RNAi construct according to the methods of the invention for reducing a cardiovascular event has experienced a myocardial infarction within one year, two years, three years, four years, or five years of receiving the first administration of the LPA RNAi construct. In one such embodiment, a patient administered an LPA RNAi construct according to the methods of the invention for reducing a cardiovascular event has experienced a myocardial infarction within one year of receiving the first administration of the LPA RNAi construct. In certain other embodiments, a patient administered an LPA RNAi construct according to the methods of the invention for reducing a cardiovascular event is hospitalized or has been recently admitted to the hospital for acute coronary syndrome or unstable angina.

In certain preferred embodiments, a patient to be administered an LPA RNAi construct according to the methods of the invention is a patient who has elevated circulating levels or concentrations of Lp(a) (e.g. elevated serum or plasma levels/concentrations of Lp(a)). A patient to be administered an LPA RNAi construct according to the methods of the invention may have baseline circulating Lp(a) levels or concentrations of about 50 nmol/L or greater, about 55 nmol/L or greater, about 60 nmol/L or greater, about 65 nmol/L or greater, about 70 nmol/L or greater, about 75 nmol/L or greater, about 100 nmol/L or greater, about 125 nmol/L or greater, about 150 nmol/L or greater, about 175 nmol/L or greater, about 200 nmol/L or greater, about 225 nmol/L or greater, or about 250 nmol/L or greater. In one embodiment, a patient is administered an LPA RNAi construct according to the methods of the invention if the patient has a serum or plasma Lp(a) level (or concentration) of about 70 nmol/L or greater prior to the first administration of the LPA RNAi construct. In another embodiment, a patient is administered an LPA RNAi construct according to the methods of the invention if the patient has a serum or plasma Lp(a) level (or concentration) of about 100 nmol/L or greater prior to the first administration of the LPA RNAi construct. In yet another embodiment, a patient is administered an LPA RNAi construct according to the methods of the invention if the patient has a serum or plasma Lp(a) level (or concentration) of about 125 nmol/L or greater prior to the first administration of the LPA RNAi construct. In still another embodiment, a patient is administered an LPA RNAi construct according to the methods of the invention if the patient has a serum or plasma Lp(a) level (or concentration) of about 150 nmol/L or greater prior to the first administration of the LPA RNAi construct. In some embodiments, a patient is administered an LPA RNAi construct according to the methods of the invention if the patient has a serum or plasma Lp(a) level (or concentration) of about 175 nmol/L or greater prior to the first administration of the LPA RNAi construct. In other embodiments, a patient is administered an LPA RNAi construct according to the methods of the invention if the patient has a serum or plasma Lp(a) level (or concentration) of about 200 nmol/L or greater prior to the first administration of the LPA RNAi construct. In certain other embodiments, a patient is administered an LPA RNAi construct according to the methods of the invention if the patient has a serum or plasma Lp(a) level (or concentration) of about 225 nmol/L or greater prior to the first administration of the LPA RNAi construct.

In less preferred embodiments in which circulating Lp(a) levels (or concentrations) are measured in mass concentration units, a patient to be administered an LPA RNAi construct according to the methods of the invention may have circulating Lp(a) levels (or concentrations) of about 30 mg/dL or greater, about 35 mg/dL or greater, about 40 mg/dL or greater, about 45 mg/dL or greater, about 50 mg/dL or greater, about 55 mg/dL or greater, about 60 mg/dL or greater, about 65 mg/dL or greater, about 70 mg/dL or greater, about 75 mg/dL or greater, about 90 mg/dL or greater, or about 100 mg/dL or greater. In one embodiment, a patient is administered an RNAi construct of the invention if the patient has a serum or plasma Lp(a) level (or concentration) of about 50 mg/dL or greater prior to the first administration of the LPA RNAi construct. In another embodiment, a patient is administered an RNAi construct of the invention if the patient has a serum or plasma Lp(a) level (or concentration) of about 60 mg/dL or greater prior to the first administration of the LPA RNAi construct. In yet another embodiment, a patient is administered an RNAi construct of the invention if the patient has a serum or plasma Lp(a) level (or concentration) of about 70 mg/dL or greater prior to the first administration of the LPA RNAi construct. In still another embodiment, a patient is administered an RNAi construct of the invention if the patient has a serum or plasma Lp(a) level (or concentration) of about 90 mg/dL or greater prior to the first administration of the LPA RNAi construct.

As discussed above, Lp(a) levels (or concentrations) can be measured in plasma or serum samples using commercially available kits, such as the Lp(a) ELISA assay kit from Mercodia AB (Uppsala, Sweden), the Lp(a) immunoturbidimetric assay from Randox Laboratories Ltd. (Crumlin, United Kingdom), or the Tina-quant® Lp(a) Gen. 2 assay from F. Hoffmann-La Roche Ltd. (Basel, Switzerland), or using other methods known in the art, such as those described Marcovina and Albers, J. Lipid Res., Vol. 57:526-537, 2016. In certain embodiments, Lp(a) levels are measured using a turbidimetric immunoassay that is standardized to detect and quantitate Lp(a) particles independent of apo(a) isoform size. In these and other embodiments, the assay used to measure Lp(a) levels is standardized against the IFCC reference material SRM2B for nmol/L (Marcovina et al., Clin. Chem., Vol. 46: 1946-1967, 2000).

In some embodiments, the patients to be administered an LPA RNAi construct according to the methods of the invention may have serum low-density lipoprotein cholesterol (LDL-C) levels within the normal range or controlled within the normal range through treatment with one or more lipid-lowering therapies. For instance, in one embodiment, a patient to be administered an LPA RNAi construct according to the methods of the invention has a serum LDL-C level of about 100 mg/dL or less prior to the first administration of the LPA RNAi construct. In another embodiment, a patient to be administered an LPA RNAi construct according to the methods of the invention has a serum LDL-C level of about 70 mg/dL or less prior to the first administration of the LPA RNAi construct. In related embodiments, the patient to be administered an LPA RNAi construct according to the methods of the invention is receiving one or more lipid-lowering therapies. Lipid-lowering therapies include, but are not limited to, PCSK9 inhibitors, such as a PCSK9 antagonist monoclonal antibody (e.g. evolocumab, alirocumab) and PCSK9-targeted siRNA (e.g. inclisiran), statins (e.g. atorvastatin, cerivastatin, fluvastatin, lovastatin, mevastatin, pitavastatin, pravastatin, rosuvastatin, simvastatin), cholesterol absorption inhibitors (e.g. ezetimibe), bempedoic acid, nicotinic acid (e.g. niacin), fabric acid (e.g. gemfibrozil, fenofibrate), bile acid sequestrants (e.g. cholestyramine, colestipol, colesevelam), LDL apheresis, or combinations thereof. In certain embodiments, the patient to be administered an LPA RNAi construct according to the methods of the invention is receiving a lipid-lowering therapy selected from the group consisting of a PCSK9 antagonist monoclonal antibody, a statin, ezetimibe, bempedoic acid, or combinations thereof.

In certain embodiments, the patients to be administered an LPA RNAi construct according to the methods of the invention have a serum triglyceride level of less than about 500 mg/dL prior to the first administration of the LPA RNAi construct. For instance, the patients may have a serum triglyceride level at baseline (e.g. prior to the first administration of the LPA RNAi construct) of less than about 400 mg/dL, less than about 375 mg/dL, less than about 350 mg/dL, less than about 325 mg/dL, less than about 300 mg/dL, less than about 275 mg/dL, less than about 250 mg/dL, less than about 225 mg/dL, less than about 200 mg/dL, less than about 175 mg/dL, or less than about 150 mg/dL. In one embodiment, the patients to be administered an LPA RNAi construct according to the methods of the invention have a serum triglyceride level of less than about 400 mg/dL prior to the first administration of the LPA RNAi construct. In another embodiment, the patients to be administered an LPA RNAi construct according to the methods of the invention have a serum triglyceride level of about 50 mg/dL to about 400 mg/dL prior to the first administration of the LPA RNAi construct. In yet another embodiment, the patients to be administered an LPA RNAi construct according to the methods of the invention have a serum triglyceride level of about 150 mg/dL to about 375 mg/dL prior to the first administration of the LPA RNAi construct.

Measurement of LDL-C, triglycerides, total cholesterol, high-density lipoprotein cholesterol (HDL-C), very-low-density lipoprotein cholesterol (VLDL-C) and other lipid biomarkers, such as apolipoprotein A1 and apolipoprotein B, can be measured with standard lipid panels using blood samples from the patients. In some embodiments, the patients fast for at least 9 hours, preferably 12 hours, prior to the sample being drawn. Thus, the levels/concentrations for the lipid biomarkers (e.g. LDL-C, triglycerides) described above can be fasting levels.

In some embodiments, the patients to be administered an LPA RNAi construct according to the methods of the invention do not have a glycated hemoglobin A1C level indicative of untreated or poorly controlled type 2 diabetes mellitus. For example, a patient to be administered an LPA RNAi construct according to the methods of the invention has a glycated hemoglobin A1C level at baseline (e.g. prior to the first administration of the LPA RNAi construct) of less than about 10.0%, less than about 9.5%, less than about 9.0%, less than about 8.5%, less than about 8.0%, less than about 7.5%, less than about 7.0%, less than about 6.5%, less than about 6.0%, or less than about 5.5%. In one embodiment, a patient to be administered an LPA RNAi construct according to the methods of the invention has a glycated hemoglobin A1C level of less than about 8.5% prior to the first administration of the LPA RNAi construct. In another embodiment, a patient to be administered an LPA RNAi construct according to the methods of the invention has a glycated hemoglobin A1C level of less than about 7.0% prior to the first administration of the LPA RNAi construct.

In other embodiments, the patients to be administered an LPA RNAi construct according to the methods of the invention do not have systolic and/or diastolic blood pressures indicative of uncontrolled hypertension. For instance, a patient to be administered an LPA RNAi construct according to the methods of the invention has an average resting systolic blood pressure at baseline (e.g. prior to the first administration of the LPA RNAi construct) of less than about 180 mmHg, less than about 160 mmHg, less than about 140 mmHg, less than about 135 mmHg, less than about 130 mmHg, less than about 125 mmHg, or less than about 120 mmHg and an average resting diastolic blood pressure at baseline (e.g. prior to the first administration of the LPA RNAi construct) of less than about 120 mmHg, less than about 110 mmHg, less than about 100 mmHg, less than about 90 mmHg, less than about 85 mmHg, or less than about 80 mmHg. In one embodiment, a patient to be administered an LPA RNAi construct according to the methods of the invention has an average systolic blood pressure less than about 180 mmHg and an average diastolic blood pressure of less than about 110 mmHg at rest prior to the first administration of the LPA RNAi construct. In another embodiment, a patient to be administered an LPA RNAi construct according to the methods of the invention has an average systolic blood pressure less than about 160 mmHg and an average diastolic blood pressure of less than about 100 mmHg at rest prior to the first administration of the LPA RNAi construct. In yet another embodiment, a patient to be administered an LPA RNAi construct according to the methods of the invention has an average systolic blood pressure less than about 140 mmHg and an average diastolic blood pressure of less than about 90 mmHg at rest prior to the first administration of the LPA RNAi construct.

In some embodiments, the patients to be administered an LPA RNAi construct according to the methods of the invention do not have signs of severe renal dysfunction. Thus, in certain embodiments, a patient to be administered an LPA RNAi construct according to the methods of the invention has an eGFR at baseline (e.g. prior to the first administration of the LPA RNAi construct) of at least about 30 mL/min/1.73 m², at least about 45 mL/min/1.73 m², at least about 60 mL/min/1.73 m², at least about 75 mL/min/1.73 m², or at least about 90 mL/min/1.73 m². In one particular embodiment, a patient to be administered an LPA RNAi construct according to the methods of the invention has an eGFR of about 30 mL/min/1.73 m 2 or greater prior to the first administration of the LPA RNAi construct.

In other embodiments, the patients to be administered an LPA RNAi construct according to the methods of the invention do not have signs of active liver disease or hepatic dysfunction. Active liver disease may be determined by measuring one or more biomarkers of hepatic function, such as those included in a liver function test or liver panel, including albumin, alkaline phosphatase (ALP), alanine transaminase (ALT), aspartate aminotransferase (AST), gamma-glutamyl transpeptidase (GGT), bilirubin, and lactate dehydrogenase (LD). In certain embodiments, a patient to be administered an LPA RNAi construct according to the methods of the invention has an ALT level at baseline (e.g. prior to the first administration of the LPA RNAi construct) of no greater than three times the upper limit of normal (ULN). In related embodiments, a patient to be administered an LPA RNAi construct according to the methods of the invention has an AST level at baseline (e.g. prior to the first administration of the LPA RNAi construct) of no greater than three times the ULN. In these and other embodiments, a patient to be administered an LPA RNAi construct according to the methods of the invention has a total bilirubin level at baseline (e.g. prior to the first administration of the LPA RNAi construct) of no greater than twice the ULN. In some embodiments, a patient to be administered an LPA RNAi construct according to the methods of the invention has at baseline (e.g. prior to the first administration of the LPA RNAi construct): (i) ALT levels of less than about 170 units/L of serum, (ii) AST levels of less than about 150 units/L of serum, and/or (iii) total bilirubin levels of less than about 2.0 mg/dL.

In one aspect, the methods of the invention comprise administering to a patient an effective amount of an LPA RNAi construct. An “effective amount” refers to an amount sufficient to treat, reduce, or ameliorate cardiovascular disease or one or more symptoms of cardiovascular disease, particularly a state or symptoms associated with cardiovascular disease, or otherwise prevent, hinder, retard or reverse the progression of cardiovascular disease or any other undesirable symptom associated with cardiovascular disease in any way whatsoever. An effective amount can also refer to an amount sufficient to reduce the occurrence or severity of sequelae resulting from a cardiovascular disease. For example, in some embodiments, an effective amount of an LPA RNAi construct is an amount sufficient to reduce the occurrence or severity of cardiovascular events, such as myocardial infarction, stroke, or revascularization of coronary, cerebral, or peripheral arteries, in patients having atherosclerosis or other cardiovascular disease.

In certain embodiments of the methods of the invention, an LPA RNAi construct is administered to a patient at a fixed dose. A “fixed dose” refers to a dose that is administered to all patients regardless of patient-specific factors, such as weight. Thus, a fixed dose is not adjusted from patient to patient based on the patient's weight. In some embodiments of the methods of the invention, the LPA RNAi construct may be administered to a patient at a fixed dose of about 9 mg to about 675 mg at a dosing interval of at least 8 weeks. For instance, the fixed dose of an LPA RNAi construct can be about 9 mg, about 10 mg, about 15 mg, about 30 mg, about 50 mg, about 60 mg, about 70 mg, about 75 mg, about 80 mg, about 90 mg, about 100 mg, about 125 mg, about 150 mg, about 175 mg, about 200 mg, about 225 mg, about 250 mg, about 300 mg, about 350 mg, about 400 mg, about 450 mg, about 500 mg, about 550 mg, about 600 mg, about 650 mg, or about 675 mg, wherein the doses are administered at a dosing interval of at least 8 weeks. Ranges between any and all of these endpoints are also contemplated, for example, the fixed dose of the LPA RNAi construct administered to a patient in the methods of the invention may be from about 10 mg to about 225 mg, about 50 mg to about 100 mg, about 150 mg to about 225 mg, about 225 mg to about 675 mg, about 75 mg to about 150 mg, about 225 mg to about 450 mg, about 75 mg to about 225 mg, about 10 mg to about 75 mg, or about 200 mg to about 300 mg, wherein the doses are administered at a dosing interval of at least 8 weeks.

Any of the doses of an LPA RNAi construct described herein are preferably administered at a dosing interval of at least 8 weeks—that is the doses are not administered to a patient more frequently than once every 8 weeks (or once every 2 months). For instance, the dosing interval may be about 8 weeks, about 12 weeks, about 16 weeks, about 20 weeks, about 24 weeks, about 28 weeks, or about 32 weeks. In certain embodiments, the dosing interval is about 12 weeks, e.g. the fixed dose of an LPA RNAi construct is administered to the patient once every 12 weeks (or once every 3 months). In certain other embodiments, the dosing interval is about 24 weeks, e.g. the fixed dose of an LPA RNAi construct is administered to the patient once every 24 weeks (or once every 6 months).

The fixed doses of the LPA RNAi construct can be administered at each dosing interval as a single bolus administration (e.g. in a single subcutaneous injection) or as two or more consecutive bolus administrations (e.g. two or more subcutaneous injections). In some embodiments, the entire amount of the fixed dose of the LPA RNAi construct is administered to the patient at each dosing interval in a single bolus injection, for example, using a pre-filled syringe or injection device as described further herein. For example, a fixed dose of 225 mg of the LPA RNAi construct can be administered to a patient as a single bolus injection of 225 mg, optionally with an autoinjector, pen injector, or pre-filled syringe containing the 225 mg dose, at each dosing interval (e.g. once every 12 weeks). In other embodiments, the entire amount of the fixed dose of the LPA RNAi construct is administered to the patient as two or more consecutive bolus injections. By way of example, a fixed dose of 225 mg of the LPA RNAi construct can be administered to the patient in three consecutive injections of 75 mg each, optionally with three injection devices (e.g. autoinjectors, pen injectors, or pre-filled syringes) each containing a 75 mg dose, at each dosing interval (e.g. once every 12 weeks). Consecutive injections given within the period of a single day are considered to be a single administration within the context of the invention. In other words, by way of example, administration of a fixed dose of 225 mg once every 12 weeks can be given either as a single bolus injection of 225 mg administered to the patient once every 12 weeks or three consecutive bolus injections of 75 mg each administered to the patient within the period of one day once every 12 weeks.

In certain embodiments of the methods of the invention, the fixed doses of the LPA RNAi construct described herein are administered once every 12 weeks or once every 3 months. In some such embodiments, the methods of the invention comprise administering to a patient an LPA RNAi construct at a fixed dose from about 10 mg to about 225 mg once every 12 weeks or once every 3 months. In other embodiments, the methods of the invention comprise administering to a patient an LPA RNAi construct at a fixed dose from about 50 mg to about 100 mg once every 12 weeks or once every 3 months. In yet other embodiments, the methods of the invention comprise administering to a patient an LPA RNAi construct at a fixed dose from about 75 mg to about 225 mg once every 12 weeks or once every 3 months. In still other embodiments, the methods of the invention comprise administering to a patient an LPA RNAi construct at a fixed dose from about 150 mg to about 225 mg once every 12 weeks or once every 3 months. In one embodiment, the methods of the invention comprise administering to a patient an LPA RNAi construct at a fixed dose of about 10 mg once every 12 weeks or once every 3 months. In another embodiment, the methods of the invention comprise administering to a patient an LPA RNAi construct at a fixed dose of about 30 mg once every 12 weeks or once every 3 months. In another embodiment, the methods of the invention comprise administering to a patient an LPA RNAi construct at a fixed dose of about 75 mg once every 12 weeks or once every 3 months. In another embodiment, the methods of the invention comprise administering to a patient an LPA RNAi construct at a fixed dose of about 100 mg once every 12 weeks or once every 3 months. In another embodiment, the methods of the invention comprise administering to a patient an LPA RNAi construct at a fixed dose of about 125 mg once every 12 weeks or once every 3 months. In yet another embodiment, the methods of the invention comprise administering to a patient an LPA RNAi construct at a fixed dose of about 150 mg once every 12 weeks or once every 3 months. In another embodiment, the methods of the invention comprise administering to a patient an LPA RNAi construct at a fixed dose of about 175 mg once every 12 weeks or once every 3 months. In another embodiment, the methods of the invention comprise administering to a patient an LPA RNAi construct at a fixed dose of about 200 mg once every 12 weeks or once every 3 months. In still another embodiment, the methods of the invention comprise administering to a patient an LPA RNAi construct at a fixed dose of about 225 mg once every 12 weeks or once every 3 months.

In certain other embodiments of the methods of the invention, the fixed doses of the LPA RNAi construct described herein are administered once every 24 weeks or once every 6 months. In some such embodiments, the methods of the invention comprise administering to a patient an LPA RNAi construct at a fixed dose from about 225 mg to about 675 mg once every 24 weeks or once every 6 months. In other embodiments, the methods of the invention comprise administering to a patient an LPA RNAi construct at a fixed dose from about 225 mg to about 450 mg once every 24 weeks or once every 6 months. In still other embodiments, the methods of the invention comprise administering to a patient an LPA RNAi construct at a fixed dose from about 200 mg to about 300 mg once every 24 weeks or once every 6 months. In one embodiment, the methods of the invention comprise administering to a patient an LPA RNAi construct at a fixed dose of about 225 mg once every 24 weeks or once every 6 months. In another embodiment, the methods of the invention comprise administering to a patient an LPA RNAi construct at a fixed dose of about 300 mg once every 24 weeks or once every 6 months. In yet another embodiment, the methods of the invention comprise administering to a patient an LPA RNAi construct at a fixed dose of about 450 mg once every 24 weeks or once every 6 months. In still another embodiment, the methods of the invention comprise administering to a patient an LPA RNAi construct at a fixed dose of about 675 mg once every 24 weeks or once every 6 months.

In some embodiments of the methods of the invention, the LPA RNAi construct is administered to the patient over the course of a set treatment period. A “treatment period” begins upon administration of a first dose of the LPA RNAi construct and ends upon administration of a final dose of the LPA RNAi construct. The treatment period may be from about 12 weeks to about 240 weeks, from about 24 weeks to about 144 weeks, from about 3 months to about 60 months, from about 6 months to about 48 months, such as about 12 weeks, about 24 weeks, about 36 weeks, about 48 weeks, about 60 weeks, about 72 weeks, about 84 weeks, about 96 weeks, about 108 weeks, about 120 weeks, about 132 weeks, about 144 weeks, about 156 weeks, about 168 weeks, about 180 weeks, about 192 weeks, about 204 weeks, about 216 weeks, about 228 weeks, about 240 weeks, about 3 months, about 6 months, about 9 months, about 12 months, about 15 months, about 18 months, about 21 months, about 24 months, about 27 months, about 30 months, about 33 months, about 36 months, about 39 months, about 42 months, about 45 months, about 48 months, about 51 months, about 54 months, about 57 months, or about 60 months. In some embodiments, the treatment period is about 48 weeks. In other embodiments, the treatment period is about 192 weeks. In yet other embodiments, the treatment period is about 12 months. In still other embodiments, the treatment period is about 48 months. In certain embodiments, the treatment period can be longer than 240 weeks or 60 months, for example, the treatment period may be greater than 5 years, such as 6, 7, 8, 9, or 10 years or more. In one particular embodiment, the LPA RNAi construct is administered for a treatment period of at least about 36 weeks and produces a statistically significant percent reduction from baseline in serum or plasma Lp(a) levels as compared to subjects not receiving the LPA RNAi construct. In another particular embodiment, the LPA RNAi construct is administered for a treatment period of at least about 48 weeks and produces a statistically significant percent reduction from baseline in serum or plasma Lp(a) levels as compared to subjects not receiving the LPA RNAi construct.

The methods described herein comprise administering to a patient an LPA RNAi construct. As used herein, the term “LPA RNAi construct” refers to an agent comprising an RNA molecule that is capable of downregulating expression of the LPA gene via an RNA interference mechanism when introduced into a cell. RNA interference is the process by which a nucleic acid molecule induces the cleavage and degradation of a target RNA molecule (e.g. messenger RNA or mRNA molecule) in a sequence-specific manner, e.g. through an RNA-induced silencing complex (RISC) pathway. In some embodiments, the LPA RNAi construct comprises a double-stranded RNA molecule comprising two antiparallel strands of contiguous nucleotides that are sufficiently complementary to each other to hybridize to form a duplex region. “Hybridize” or “hybridization” refers to the pairing of complementary polynucleotides, typically via hydrogen bonding (e.g. Watson-Crick, Hoogsteen or reversed Hoogsteen hydrogen bonding) between complementary bases in the two polynucleotides. The strand comprising a region having a sequence that is substantially complementary to a target LPA sequence (e.g. target LPA mRNA) is referred to as the “antisense strand.” The “sense strand” refers to the strand that includes a region that is substantially complementary to a region of the antisense strand. In some embodiments, the sense strand may comprise a region that has a sequence that is substantially identical to the target sequence.

A double-stranded RNA molecule may include chemical modifications to ribonucleotides, including modifications to the ribose sugar, base, or backbone components of the ribonucleotides, such as those described herein or known in the art. Any such modifications, as used in a double-stranded RNA molecule (e.g. siRNA, shRNA, or the like), are encompassed by the term “double-stranded RNA” for the purposes of this disclosure.

As used herein, a first sequence is “complementary” to a second sequence if a polynucleotide comprising the first sequence can hybridize to a polynucleotide comprising the second sequence to form a duplex region under certain conditions, such as physiological conditions. Other such conditions can include moderate or stringent hybridization conditions, which are known to those of skill in the art. A first sequence is considered to be fully complementary (100% complementary) to a second sequence if a polynucleotide comprising the first sequence base pairs with a polynucleotide comprising the second sequence over the entire length of one or both nucleotide sequences without any mismatches. A sequence is “substantially complementary” to a target sequence if the sequence is at least about 80%, 85%, 90%, 95%, 96%, 97%, 98%, or 99% complementary to a target sequence. Percent complementarity can be calculated by dividing the number of bases in a first sequence that are complementary to bases at corresponding positions in a second or target sequence by the total length of the first sequence. A sequence may also be said to be substantially complementary to another sequence if there are no more than 5, 4, 3, or 2 mismatches over a 30 base pair duplex region when the two sequences are hybridized. Generally, if any nucleotide overhangs, as defined herein, are present, the sequence of such overhangs is not considered in determining the degree of complementarity between two sequences. By way of example, a sense strand of 21 nucleotides in length and an antisense strand of 21 nucleotides in length that hybridize to form a 19 base pair duplex region with a 2-nucleotide overhang at the 3′ end of each strand would be considered to be fully complementary as the term is used herein.

In some embodiments, a region of the antisense strand comprises a sequence that is substantially or fully complementary to a region of the target LPA RNA sequence (e.g. LPA mRNA). In such embodiments, the sense strand may comprise a sequence that is fully complementary to the sequence of the antisense strand. In other such embodiments, the sense strand may comprise a sequence that is substantially complementary to the sequence of the antisense strand, e.g. having 1, 2, 3, 4, or 5 mismatches in the duplex region formed by the sense and antisense strands. In certain embodiments, it is preferred that any mismatches occur within the terminal regions (e.g. within 6, 5, 4, 3, or 2 nucleotides of the 5′ and/or 3′ ends of the strands). In one embodiment, any mismatches in the duplex region formed from the sense and antisense strands occur within 6, 5, 4, 3, or 2 nucleotides of the 5′ end of the antisense strand.

In certain embodiments, the sense strand and antisense strand of the double-stranded RNA may be two separate molecules that hybridize to form a duplex region but are otherwise unconnected. Such double-stranded RNA molecules formed from two separate strands are referred to as “small interfering RNAs” or “short interfering RNAs” (siRNAs). Thus, in some embodiments, the LPA RNAi constructs employed in the methods of the invention comprise an siRNA.

In other embodiments, the sense strand and the antisense strand that hybridize to form a duplex region may be part of a single RNA molecule, i.e. the sense and antisense strands are part of a self-complementary region of a single RNA molecule. In such cases, a single RNA molecule comprises a duplex region (also referred to as a stem region) and a loop region. The 3′ end of the sense strand is connected to the 5′ end of the antisense strand by a contiguous sequence of unpaired nucleotides, which will form the loop region. The loop region is typically of a sufficient length to allow the RNA molecule to fold back on itself such that the antisense strand can base pair with the sense strand to form the duplex or stem region. The loop region can comprise from about 3 to about 25, from about 5 to about 15, or from about 8 to about 12 unpaired nucleotides. Such RNA molecules with at least partially self-complementary regions are referred to as “short hairpin RNAs” (shRNAs). In certain embodiments, the LPA RNAi constructs used in the methods of the invention comprise a shRNA. The length of a single, at least partially self-complementary RNA molecule can be from about 40 nucleotides to about 100 nucleotides, from about 45 nucleotides to about 85 nucleotides, or from about 50 nucleotides to about 60 nucleotides and comprise a duplex region and loop region each having the lengths recited herein.

The LPA RNAi constructs employed in the methods of the invention comprise a sense strand and an antisense strand, wherein the antisense strand comprises a region having a sequence that is substantially or fully complementary to an LPA messenger RNA (mRNA) sequence. As used herein, a “LPA mRNA sequence” refers to any messenger RNA sequence, including allelic variants and splice variants, encoding an apo(a) protein, including apo(a) protein variants or isoforms from any species (e.g. non-human primate, human). The LPA gene (also known as AK38, APOA, and LP) encodes the apo(a) protein, which is a primary component of the low-density lipoprotein particle known as lipoprotein (a) or Lp(a). In humans, the LPA gene is found on chromosome 6 at locus 6q25.3-q26. The LPA gene is highly polymorphic with alleles of the gene differing in numbers of copies of the kringle IV type 2 (KIV-2) domain, which can range from two to over 40 copies among individuals (see, e.g., Kronenberg and Utermann, J. Intern. Med., Vol. 273:6-30, 2013).

An LPA mRNA sequence also includes the transcript sequence expressed as its complementary DNA (cDNA) sequence. A cDNA sequence refers to the sequence of an mRNA transcript expressed as DNA bases (e.g. guanine, adenine, thymine, and cytosine) rather than RNA bases (e.g. guanine, adenine, uracil, and cytosine). Thus, the antisense strand of the LPA RNAi constructs used in the methods of the invention may comprise a region having a sequence that is substantially or fully complementary to a target LPA mRNA sequence or LPA cDNA sequence. An LPA mRNA or cDNA sequence can include, but is not limited to, any LPA mRNA or cDNA sequence selected from the NCBI Reference sequences NM 005577.4 (human), XM 015448520.1 (cynomolgus monkey), XM 028847001.1 (rhesus monkey), XM 024357489.1 (chimpanzee), and XM 031012244.1 (gorilla). In certain embodiments, the LPA mRNA sequence is the human transcript listed in the NCBI database as Reference Sequence NM 005577.4.

The sense strand of the LPA RNAi construct typically comprises a sequence that is sufficiently complementary to the sequence of the antisense strand such that the two strands hybridize under physiological conditions to form a duplex region. A “duplex region” refers to the region in two complementary or substantially complementary polynucleotides that form base pairs with one another, either by Watson-Crick base pairing or other hydrogen bonding interaction, to create a duplex between the two polynucleotides. The duplex region of the LPA RNAi construct should be of sufficient length to allow the LPA RNAi construct to enter the RNA interference pathway, e.g. by engaging the Dicer enzyme and/or the RISC complex. For instance, in some embodiments, the duplex region is about 15 to about 30 base pairs in length. Other lengths for the duplex region within this range are also suitable, such as about 15 to about 28 base pairs, about 15 to about 26 base pairs, about 15 to about 24 base pairs, about 15 to about 22 base pairs, about 17 to about 28 base pairs, about 17 to about 26 base pairs, about 17 to about 24 base pairs, about 17 to about 23 base pairs, about 17 to about 21 base pairs, about 19 to about 25 base pairs, about 19 to about 23 base pairs, or about 19 to about 21 base pairs. In certain embodiments, the duplex region is about 17 to about 26 base pairs in length. In other embodiments, the duplex region is about 19 to about 21 base pairs in length. In one embodiment, the duplex region is about 19 base pairs in length. In another embodiment, the duplex region is about 21 base pairs in length.

For embodiments in which the sense strand and antisense strand are two separate molecules (e.g. RNAi construct comprises an siRNA), the sense strand and antisense strand need not be the same length as the length of the duplex region. For instance, one or both strands may be longer than the duplex region and have one or more unpaired nucleotides or mismatches flanking the duplex region. Thus, in some embodiments, the RNAi construct comprises at least one nucleotide overhang. As used herein, a “nucleotide overhang” refers to the unpaired nucleotide or nucleotides that extend beyond the duplex region at the terminal ends of the strands. Nucleotide overhangs are typically created when the 3′ end of one strand extends beyond the 5′ end of the other strand or when the 5′ end of one strand extends beyond the 3′ end of the other strand. The length of a nucleotide overhang is generally between 1 and 6 nucleotides, 1 and 5 nucleotides, 1 and 4 nucleotides, 1 and 3 nucleotides, 2 and 6 nucleotides, 2 and 5 nucleotides, or 2 and 4 nucleotides. In some embodiments, the nucleotide overhang comprises 1, 2, 3, 4, 5, or 6 nucleotides. In one particular embodiment, the nucleotide overhang comprises 1 to 4 nucleotides. In certain embodiments, the nucleotide overhang comprises 2 nucleotides. In certain other embodiments, the nucleotide overhang comprises a single nucleotide. When a nucleotide overhang is present in the antisense strand, the nucleotides in the overhang can be complementary to the target gene sequence, form a mismatch with the target gene sequence, or comprise some other sequence (e.g. polypyrimidine or polypurine sequence, such as UU, TT, AA, GG, etc.).

The nucleotide overhang can be at the 5′ end or 3′ end of one or both strands. For example, in one embodiment, the LPA RNAi construct comprises a nucleotide overhang at the 5′ end and the 3′ end of the antisense strand. In another embodiment, the LPA RNAi construct comprises a nucleotide overhang at the 5′ end and the 3′ end of the sense strand. In some embodiments, the LPA RNAi construct comprises a nucleotide overhang at the 5′ end of the sense strand and the 5′ end of the antisense strand. In other embodiments, the LPA RNAi construct comprises a nucleotide overhang at the 3′ end of the sense strand and the 3′ end of the antisense strand.

The RNAi constructs may comprise a nucleotide overhang at one end of the double-stranded RNA molecule and a blunt end at the other. A “blunt end” means that the sense strand and antisense strand are fully base-paired at the end of the molecule and there are no unpaired nucleotides that extend beyond the duplex region. In some embodiments, the LPA RNAi construct comprises a nucleotide overhang at the 3′ end of the sense strand and a blunt end at the 5′ end of the sense strand and 3′ end of the antisense strand. In other embodiments, the LPA RNAi construct comprises a nucleotide overhang at the 3′ end of the antisense strand and a blunt end at the 5′ end of the antisense strand and the 3′ end of the sense strand. In certain embodiments, the LPA RNAi construct comprises a blunt end at both ends of the double-stranded RNA molecule. In such embodiments, the sense strand and antisense strand have the same length and the duplex region is the same length as the sense and antisense strands (i.e. the molecule is double-stranded over its entire length).

The sense strand and antisense strand in the LPA RNAi constructs used in the methods of the invention can each independently be about 15 to about 30 nucleotides in length, about 19 to about 30 nucleotides in length, about 18 to about 28 nucleotides in length, about 19 to about 27 nucleotides in length, about 19 to about 25 nucleotides in length, about 19 to about 23 nucleotides in length, about 19 to about 21 nucleotides in length, about 21 to about 25 nucleotides in length, or about 21 to about 23 nucleotides in length. In certain embodiments, the sense strand and antisense strand are each independently about 18, about 19, about 20, about 21, about 22, about 23, about 24, or about 25 nucleotides in length. In some embodiments, the sense strand and antisense strand have the same length but form a duplex region that is shorter than the strands such that the LPA RNAi construct has two nucleotide overhangs. For instance, in one embodiment, the LPA RNAi construct comprises (i) a sense strand and an antisense strand that are each 21 nucleotides in length, (ii) a duplex region that is 19 base pairs in length, and (iii) nucleotide overhangs of 2 unpaired nucleotides at both the 3′ end of the sense strand and the 3′ end of the antisense strand. In another embodiment, the LPA RNAi construct comprises (i) a sense strand and an antisense strand that are each 23 nucleotides in length, (ii) a duplex region that is 21 base pairs in length, and (iii) nucleotide overhangs of 2 unpaired nucleotides at both the 3′ end of the sense strand and the 3′ end of the antisense strand. In other embodiments, the sense strand and antisense strand have the same length and form a duplex region over their entire length such that there are no nucleotide overhangs on either end of the double-stranded molecule. In one particular embodiment, the LPA RNAi construct employed in the methods of the invention is blunt ended and comprises (i) a sense strand and an antisense strand, each of which is 21 nucleotides in length, and (ii) a duplex region that is 21 base pairs in length. In another particular embodiment, the LPA RNAi construct employed in the methods of the invention is blunt ended and comprises (i) a sense strand and an antisense strand, each of which is 19 nucleotides in length, and (ii) a duplex region that is 19 base pairs in length.

In other embodiments, the sense strand or the antisense strand is longer than the other strand and the two strands form a duplex region having a length equal to that of the shorter strand such that the LPA RNAi construct comprises at least one nucleotide overhang. For example, in one embodiment, the LPA RNAi construct employed in the methods of the invention comprises (i) a sense strand that is 19 nucleotides in length, (ii) an antisense strand that is 21 nucleotides in length, (iii) a duplex region of 19 base pairs in length, and (iv) a nucleotide overhang of 2 unpaired nucleotides at the 3′ end of the antisense strand. In another embodiment, the LPA RNAi construct employed in the methods of the invention comprises (i) a sense strand that is 21 nucleotides in length, (ii) an antisense strand that is 23 nucleotides in length, (iii) a duplex region of 21 base pairs in length, and (iv) a nucleotide overhang of 2 unpaired nucleotides at the 3′ end of the antisense strand.

The LPA RNAi constructs used in the methods of the invention may comprise one or more modified nucleotides. A “modified nucleotide” refers to a nucleotide that has one or more chemical modifications to the nucleoside, nucleobase, pentose ring, or phosphate group. As used herein, modified nucleotides do not encompass ribonucleotides containing adenosine monophosphate, guanosine monophosphate, uridine monophosphate, and cytidine monophosphate. However, the LPA RNAi constructs may comprise combinations of modified nucleotides and ribonucleotides. Incorporation of modified nucleotides into one or both strands of double-stranded RNA molecules can improve the in vivo stability of the RNA molecules, e.g., by reducing the molecules' susceptibility to nucleases and other degradation processes. The potency of LPA RNAi constructs for reducing expression of the LPA gene can also be enhanced by incorporation of modified nucleotides.

In certain embodiments, the modified nucleotides have a modification of the ribose sugar. These sugar modifications can include modifications at the 2′ and/or 5′ position of the pentose ring as well as bicyclic sugar modifications. A 2′-modified nucleotide refers to a nucleotide having a pentose ring with a substituent at the 2′ position other than OH. Such 2′-modifications include, but are not limited to, 2′-H (e.g. deoxyribonucleotides), 2′-O-alkyl (e.g. O—C₁-C₁₀ or O-C₁-C₁₀ substituted alkyl), 2′-O-allyl (O—CH₂CH═CH₂), 2′-C-allyl, 2′-deoxy-2′-fluoro (also referred to as 2′-F or 2′-fluoro), 2′-O-methyl (OCH₃), 2′-O-methoxyethyl (O—(CH₂)₂OCH₃), 2′-OCF₃, 2′-O(CH₂)₂SCH₃, 2′-O-aminoalkyl, 2′-amino (e.g. NH₂), 2′-O-ethylamine, and 2′-azido. Modifications at the 5′ position of the pentose ring include, but are not limited to, 5′-methyl (R or S); 5′-vinyl, and 5′-methoxy.

A “bicyclic sugar modification” refers to a modification of the pentose ring where a bridge connects two atoms of the ring to form a second ring resulting in a bicyclic sugar structure. In some embodiments the bicyclic sugar modification comprises a bridge between the 4′ and 2′ carbons of the pentose ring. Nucleotides comprising a sugar moiety with a bicyclic sugar modification are referred to herein as bicyclic nucleic acids or BNAs. Exemplary bicyclic sugar modifications include, but are not limited to, α-L-Methyleneoxy (4′-CH₂—O-2′) bicyclic nucleic acid (BNA); β-D-Methyleneoxy (4′-CH₂—O-2′) BNA (also referred to as a locked nucleic acid or LNA); Ethyleneoxy (4′-(CH₂)₂—O-2′) BNA; Aminooxy (4′-CH₂—O—N(R)-2′) BNA; Oxyamino (4′-CH₂—N(R)—O-2′) BNA; Methyl(methyleneoxy) (4′-CH(CH₃)—O-2′) BNA (also referred to as constrained ethyl or cEt); methylene-thio (4′-CH₂—S-2′) BNA; methylene-amino (4′-CH₂—N(R)-2′) BNA; methyl carbocyclic (4′-CH₂—CH(CH₃)-2′) BNA; propylene carbocyclic (4′-(CH₂)₃-2′) BNA; and Methoxy(ethyleneoxy) (4′-CH(CH₂OMe)-O-2′) BNA (also referred to as constrained MOE or cMOE). These and other sugar-modified nucleotides that can be incorporated into the LPA RNAi constructs used in the methods of the invention are described in U.S. Pat. No. 9,181,551, U.S. Patent Publication No. 2016/0122761, and Deleavey and Damha, Chemistry and Biology, Vol. 19: 937-954, 2012.

In some embodiments, the LPA RNAi constructs comprise one or more 2′-fluoro modified nucleotides, 2′-O-methyl modified nucleotides, 2′-O-methoxyethyl modified nucleotides, 2′-O-alkyl modified nucleotides, 2′-O-allyl modified nucleotides, bicyclic nucleic acids (BNAs), deoxyribonucleotides, or combinations thereof. In certain embodiments, the LPA RNAi constructs comprise one or more 2′-fluoro modified nucleotides, 2′-O-methyl modified nucleotides, 2′-O-methoxyethyl modified nucleotides, deoxyribonucleotides, or combinations thereof. In one particular embodiment, the LPA RNAi constructs used in the methods of the invention comprise one or more 2′-fluoro modified nucleotides, 2′-O-methyl modified nucleotides, deoxyribonucleotides, or combinations thereof. In some such embodiments, the deoxyribonucleotide may be the terminal nucleotide at the 3′ end and/or 5′ end of the sense strand or antisense strand. In such embodiments in which the deoxyribonucleotide is a terminal nucleotide, it may be an inverted nucleotide—that is, linked to the adjacent nucleotide through a 3′-3′ internucleotide linkage (when on the 3′ end of a strand) or through a 5′-5′ internucleotide linkage (when on the 5′ end of a strand) rather than the natural 3′-5′ internucleotide linkage.

Both the sense and antisense strands of the LPA RNAi constructs can comprise one or multiple modified nucleotides. For instance, in some embodiments, the sense strand comprises 1, 2, 3, 4, 5, 6, 7, 8, 9, 10 or more modified nucleotides. In certain embodiments, all nucleotides in the sense strand are modified nucleotides. In some embodiments, the antisense strand comprises 1, 2, 3, 4, 5, 6, 7, 8, 9, 10 or more modified nucleotides. In other embodiments, all nucleotides in the antisense strand are modified nucleotides. In certain other embodiments, all nucleotides in the sense strand and all nucleotides in the antisense strand are modified nucleotides. In these and other embodiments, the modified nucleotides can be 2′-fluoro modified nucleotides, 2′-O-methyl modified nucleotides, or combinations thereof.

In certain embodiments, the modified nucleotides incorporated into one or both of the strands of the LPA RNAi constructs used in the methods of the invention have a modification of the nucleobase (also referred to herein as “base”). A “modified nucleobase” or “modified base” refers to a base other than the naturally occurring purine bases adenine (A) and guanine (G) and pyrimidine bases thymine (T), cytosine (C), and uracil (U). Modified nucleobases can be synthetic or naturally occurring modifications and include, but are not limited to, universal bases, 5-methylcytosine (5-me-C), 5-hydroxymethyl cytosine, xanthine (X), hypoxanthine (I), 2-aminoadenine, 6-methyladenine, 6-methylguanine, and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

In some embodiments, the modified base is a universal base. A “universal base” refers to a base analog that indiscriminately forms base pairs with all of the natural bases in RNA and DNA without altering the double helical structure of the resulting duplex region. Universal bases are known to those of skill in the art and include, but are not limited to, inosine, C-phenyl, C-naphthyl and other aromatic derivatives, azole carboxamides, and nitroazole derivatives, such as 3-nitropyrrole, 4-nitroindole, 5-nitroindole, and 6-nitroindole.

Other suitable modified bases that can be incorporated into the LPA RNAi constructs include those described in Herdewijn, Antisense Nucleic Acid Drug Dev., Vol. 10: 297-310, 2000 and Peacock et al., J. Org. Chem., Vol. 76: 7295-7300, 2011. The skilled person is well aware that guanine, cytosine, adenine, thymine, and uracil may be replaced by other nucleobases, such as the modified nucleobases described above, without substantially altering the base pairing properties of a polynucleotide comprising a nucleotide bearing such replacement nucleobase.

In some embodiments, the sense and antisense strands of the LPA RNAi constructs used in the methods of the invention may comprise one or more abasic nucleotides. An “abasic nucleotide” or “abasic nucleoside” is a nucleotide or nucleoside that lacks a nucleobase at the 1′ position of the ribose sugar. In certain embodiments, the abasic nucleotides are incorporated into the terminal ends of the sense and/or antisense strands of the RNAi constructs. In one embodiment, the sense strand comprises an abasic nucleotide as the terminal nucleotide at its 3′ end, its 5′ end, or both its 3′ and 5′ ends. In another embodiment, the antisense strand comprises an abasic nucleotide as the terminal nucleotide at its 3′ end, its 5′ end, or both its 3′ and 5′ ends. In such embodiments in which the abasic nucleotide is a terminal nucleotide, it may be an inverted nucleotide—that is, linked to the adjacent nucleotide through a 3′-3′ internucleotide linkage (when on the 3′ end of a strand) or through a 5′-5′ internucleotide linkage (when on the 5′ end of a strand) rather than the natural 3′-5′ internucleotide linkage. Abasic nucleotides may also comprise a sugar modification, such as any of the sugar modifications described above. In certain embodiments, abasic nucleotides comprise a 2′-modification, such as a 2′-fluoro modification, 2′-modification, or a 2′-H (deoxy) modification. In one embodiment, the abasic nucleotide comprises a 2′-O-methyl modification. In another embodiment, the abasic nucleotide comprises a 2′-H modification (i.e. a deoxy abasic nucleotide).

The LPA RNAi constructs used in the methods of the invention may also comprise one or more modified internucleotide linkages. As used herein, the term “modified internucleotide linkage” refers to an internucleotide linkage other than the natural 3′ to 5′ phosphodiester linkage. In some embodiments, the modified internucleotide linkage is a phosphorous-containing internucleotide linkage, such as a phosphotriester, aminoalkylphosphotriester, an alkylphosphonate (e.g. methylphosphonate, 3′-alkylene phosphonate), a phosphinate, a phosphoramidate (e.g. 3′-amino phosphoramidate and aminoalkylphosphoramidate), a phosphorothioate (P═S), a chiral phosphorothioate, a phosphorodithioate, a thionophosphoramidate, a thionoalkylphosphonate, a thionoalkylphosphotriester, and a boranophosphate. In one embodiment, a modified internucleotide linkage is a 2′ to 5′ phosphodiester linkage. In other embodiments, the modified internucleotide linkage is a non-phosphorous-containing internucleotide linkage and thus can be referred to as a modified internucleoside linkage. Such non-phosphorous-containing linkages include, but are not limited to, morpholino linkages (formed in part from the sugar portion of a nucleoside); siloxane linkages (—O—Si(H)₂—O—); sulfide, sulfoxide and sulfone linkages; formacetyl and thioformacetyl linkages; alkene containing backbones; sulfamate backbones; methylenemethylimino (—CH₂—N(CH₃)—O—CH₂—) and methylenehydrazino linkages; sulfonate and sulfonamide linkages; amide linkages; and others having mixed N, O, S and CH₂ component parts. In one embodiment, the modified internucleoside linkage is a peptide-based linkage (e.g. aminoethylglycine) to create a peptide nucleic acid or PNA, such as those described in U.S. Pat. Nos. 5,539,082; 5,714,331; and 5,719,262. Other suitable modified internucleotide and internucleoside linkages that may be employed in the LPA RNAi constructs are described in U.S. Pat. Nos. 6,693,187, 9,181,551, U.S. Patent Publication No. 2016/0122761, and Deleavey and Damha, Chemistry and Biology, Vol. 19: 937-954, 2012.

In certain embodiments, the LPA RNAi constructs used in the methods of the invention comprise one or more phosphorothioate internucleotide linkages. The phosphorothioate internucleotide linkages may be present in the sense strand, antisense strand, or both strands of the LPA RNAi constructs. For instance, in some embodiments, the sense strand comprises 1, 2, 3, 4, 5, 6, 7, 8, or more phosphorothioate internucleotide linkages. In other embodiments, the antisense strand comprises 1, 2, 3, 4, 5, 6, 7, 8, or more phosphorothioate internucleotide linkages. In still other embodiments, both strands comprise 1, 2, 3, 4, 5, 6, 7, 8, or more phosphorothioate internucleotide linkages. The LPA RNAi constructs can comprise one or more phosphorothioate internucleotide linkages at the 3′-end, the 5′-end, or both the 3′- and 5′-ends of the sense strand, the antisense strand, or both strands. For instance, in certain embodiments, the LPA RNAi construct comprises about 1 to about 6 or more (e.g., about 1, 2, 3, 4, 5, 6 or more) consecutive phosphorothioate internucleotide linkages at the 3′-end of the sense strand, the antisense strand, or both strands. In other embodiments, the LPA RNAi construct comprises about 1 to about 6 or more (e.g., about 1, 2, 3, 4, 5, 6 or more) consecutive phosphorothioate internucleotide linkages at the 5′-end of the sense strand, the antisense strand, or both strands. In any of the embodiments in which one or both strands comprise one or more phosphorothioate internucleotide linkages, the remaining internucleotide linkages within the strands can be the natural 3′ to 5′ phosphodiester linkages. For instance, in some embodiments, each internucleotide linkage of the sense and antisense strands is selected from phosphodiester and phosphorothioate, wherein at least one internucleotide linkage is a phosphorothioate.

In some embodiments of the RNAi constructs of the invention, the 5′ end of the sense strand, antisense strand, or both the antisense and sense strands comprises a phosphate moiety. As used herein, the term “phosphate moiety” refers to a terminal phosphate group that includes unmodified phosphates (—O—P═O)(OH)OH) as well as modified phosphates. Modified phosphates include phosphates in which one or more of the O and OH groups is replaced with H, O, S, N(R) or alkyl where R is H, an amino protecting group or unsubstituted or substituted alkyl. Exemplary phosphate moieties include, but are not limited to, 5′-monophosphate; 5′-diphosphate; 5′-triphosphate; 5′-guanosine cap (7-methylated or non-methylated); 5′-adenosine cap or any other modified or unmodified nucleotide cap structure; 5′-monothiophosphate (phosphorothioate); 5′-monodithiophosphate (phosphorodithioate); 5′-alpha-thiotriphosphate; 5′-gamma-thiotriphosphate, 5′-phosphoramidates; 5′-vinylphosphates; 5′-alkylphosphonates (e.g., alkyl=methyl, ethyl, isopropyl, propyl, etc.); 5′-cyclopropyl phosphonate, and 5′-alkyletherphosphonates (e.g., alkylether=methoxymethyl, ethoxymethyl, etc.).

The modified nucleotides that can be incorporated into the LPA RNAi constructs suitable for use in the methods of the invention may have more than one chemical modification described herein. For instance, the modified nucleotide may have a modification to the ribose sugar as well as a modification to the nucleobase. By way of example, a modified nucleotide may comprise a 2′ sugar modification (e.g. 2′-fluoro or 2′-O-methyl) and comprise a modified base (e.g. 5-methyl cytosine or pseudouracil). In other embodiments, the modified nucleotide may comprise a sugar modification in combination with a modification to the 5′ phosphate that would create a modified internucleotide or internucleoside linkage when the modified nucleotide was incorporated into a polynucleotide. For instance, in some embodiments, the modified nucleotide may comprise a sugar modification, such as a 2′-fluoro modification, a 2′-O-methyl modification, or a bicyclic sugar modification, as well as a 5′ phosphorothioate group. Accordingly, in some embodiments, one or both strands of the RNAi constructs of the invention comprise a combination of 2′ modified nucleotides or BNAs and phosphorothioate internucleotide linkages. In certain embodiments, both the sense and antisense strands of the RNAi constructs of the invention comprise a combination of 2′-fluoro modified nucleotides, 2′-O-methyl modified nucleotides, and phosphorothioate internucleotide linkages.

The LPA gene is expressed predominantly in the liver. Thus, in certain embodiments, it is desirable to specifically deliver the LPA RNAi constructs to liver cells. Accordingly, in some embodiments, the LPA RNAi constructs employed in the methods of the invention may comprise a targeting moiety to direct the LPA RNAi construct specifically to liver cells (e.g. hepatocytes) using various approaches as described in more detail below. In certain embodiments, the LPA RNAi constructs comprise a targeting moiety that comprises a ligand that binds to the surface-expressed asialoglycoprotein receptor (ASGR) or component thereof (e.g. ASGR1, ASGR2).

In some embodiments, LPA RNAi constructs can be specifically targeted to the liver by employing ligands that bind to or interact with proteins expressed on the surface of liver cells. For example, in certain embodiments, the ligands may comprise antigen binding proteins (e.g. antibodies or binding fragments thereof (e.g. Fab, scFv)) that specifically bind to a receptor expressed on hepatocytes, such as the asialoglycoprotein receptor and the LDL receptor. In one particular embodiment, the ligand comprises an antibody or binding fragment thereof that specifically binds to ASGR1 and/or ASGR2. In another embodiment, the ligand comprises a Fab fragment of an antibody that specifically binds to ASGR1 and/or ASGR2. A “Fab fragment” is comprised of one immunoglobulin light chain (i.e. light chain variable region (VL) and constant region (CL)) and the CH1 region and variable region (VH) of one immunoglobulin heavy chain. In another embodiment, the ligand comprises a single-chain variable antibody fragment (scFv fragment) of an antibody that specifically binds to ASGR1 and/or ASGR2. An “scFv fragment” comprises the VH and VL regions of an antibody, wherein these regions are present in a single polypeptide chain, and optionally comprising a peptide linker between the VH and VL regions that enables the Fv to form the desired structure for antigen binding. Exemplary antibodies and binding fragments thereof that specifically bind to ASGR1 that can be used as asialoglycoprotein receptor ligands in the targeting moieties of the LPA RNAi constructs employed in the methods of the invention are described in WIPO Publication No. WO 2017/058944, which is hereby incorporated by reference in its entirety. Other antibodies or binding fragments thereof that specifically bind to ASGR1, LDL receptor, or other liver surface-expressed proteins suitable for use as targeting moieties in the LPA RNAi constructs are commercially available.

In certain embodiments, the targeting moiety comprises a carbohydrate. A “carbohydrate” refers to a compound made up of one or more monosaccharide units having at least 6 carbon atoms (which can be linear, branched or cyclic) with an oxygen, nitrogen or sulfur atom bonded to each carbon atom. Carbohydrates include, but are not limited to, the sugars (e.g., monosaccharides, disaccharides, trisaccharides, tetrasaccharides, and oligosaccharides containing from about 4, 5, 6, 7, 8, or 9 monosaccharide units), and polysaccharides, such as starches, glycogen, cellulose and polysaccharide gums. In some embodiments, the carbohydrate incorporated into the targeting moiety is a monosaccharide selected from a pentose, hexose, or heptose and di- and tri-saccharides including such monosaccharide units. In other embodiments, the carbohydrate incorporated into the targeting moiety is an amino sugar, such as galactosamine, glucosamine, N-acetylgalactosamine, and N-acetylglucosamine.

In some embodiments, the targeting moiety comprises an asialoglycoprotein receptor ligand that comprises glucose, galactose, galactosamine, glucosamine, N-acetylglucosamine, N-acetyl-galactosamine, or a derivative of any of the foregoing. In particular embodiments, the asialoglycoprotein receptor ligand comprises N-acetyl-galactosamine (GalNAc) or a derivative thereof. Ligands comprising glucose, galactose, and GalNAc are particularly effective in targeting compounds to liver cells because such ligands bind to the ASGR expressed on the surface of hepatocytes. See, e.g., D'Souza and Devarajan, J. Control Release, Vol. 203: 126-139, 2015. Examples of GalNAc- or galactose-containing ligands that can be incorporated into the targeting moiety of the LPA RNAi constructs employed in the methods of the invention are described in U.S. Pat. Nos. 7,491,805; 8,106,022; 8,877,917; and 10,246,709; U.S. Patent Publication No. 20030130186; and WIPO Publication No. WO 2013166155, all of which are hereby incorporated by reference in their entireties.

In certain embodiments, the targeting moiety in the LPA RNAi construct comprises a multivalent carbohydrate moiety. As used herein, a “multivalent carbohydrate moiety” refers to a moiety comprising two or more carbohydrate units capable of independently binding or interacting with other molecules. For example, a multivalent carbohydrate moiety comprises two or more binding domains comprised of carbohydrates that can bind to two or more different molecules or two or more different sites on the same molecule. The valency of the carbohydrate moiety denotes the number of individual binding domains within the carbohydrate moiety. For instance, the terms “monovalent,” “bivalent,” “trivalent,” and “tetravalent” with reference to the carbohydrate moiety refer to carbohydrate moieties with one, two, three, and four binding domains, respectively. The multivalent carbohydrate moiety may comprise a multivalent lactose moiety, a multivalent galactose moiety, a multivalent glucose moiety, a multivalent N-acetyl-galactosamine moiety, a multivalent N-acetyl-glucosamine moiety, a multivalent mannose moiety, or a multivalent fucose moiety. In some embodiments, the targeting moiety comprises a multivalent galactose moiety. In other embodiments, the targeting moiety comprises a multivalent N-acetyl-galactosamine moiety. In these and other embodiments, the multivalent carbohydrate moiety can be bivalent, trivalent, or tetravalent. In such embodiments, the multivalent carbohydrate moiety can be bi-antennary or tri-antennary. In one particular embodiment, the multivalent N-acetyl-galactosamine moiety is trivalent or tetravalent. In another particular embodiment, the multivalent galactose moiety is trivalent or tetravalent.

The targeting moiety can be attached or conjugated to the RNA molecule of the LPA RNAi construct directly or indirectly. For instance, in some embodiments, the targeting moiety is covalently attached directly to the sense or antisense strand of the LPA RNAi construct. In other embodiments, the targeting moiety is covalently attached via a linker to the sense or antisense strand of the LPA RNAi construct. The targeting moiety can be attached to nucleobases, sugar moieties, or internucleotide linkages of polynucleotides (e.g. sense strand or antisense strand) of the LPA RNAi constructs used in the methods of the invention. Conjugation or attachment to purine nucleobases or derivatives thereof can occur at any position including, endocyclic and exocyclic atoms. In certain embodiments, the 2-, 6-, 7-, or 8-positions of a purine nucleobase are attached to a targeting moiety. Conjugation or attachment to pyrimidine nucleobases or derivatives thereof can also occur at any position. In some embodiments, the 2-, 5-, and 6-positions of a pyrimidine nucleobase can be attached to a targeting moiety. Conjugation or attachment to sugar moieties of nucleotides can occur at any carbon atom. Exemplary carbon atoms of a sugar moiety that can be attached to a targeting moiety include the 2′, 3′, and 5′ carbon atoms. The 1′ position can also be attached to a targeting moiety, such as in an abasic nucleotide. Internucleotide linkages can also support targeting moiety attachments. For phosphorus-containing linkages (e.g., phosphodiester, phosphorothioate, phosphorodithiotate, phosphoroamidate, and the like), the targeting moiety can be attached directly to the phosphorus atom or to an O, N, or S atom bound to the phosphorus atom. For amine- or amide-containing internucleoside linkages (e.g., PNA), the targeting moiety can be attached to the nitrogen atom of the amine or amide or to an adjacent carbon atom.

In some embodiments, the targeting moiety may be attached to the 3′ or 5′ end of either the sense or antisense strand. In certain preferred embodiments, the targeting moiety is covalently attached to the 5′ end of the sense strand. In such embodiments, the targeting moiety is attached to the 5′-terminal nucleotide of the sense strand. In these and other embodiments, the targeting moiety is attached at the 5′-position of the 5′-terminal nucleotide of the sense strand. In embodiments in which an inverted abasic nucleotide or inverted deoxyribonucleotide is the 5′-terminal nucleotide of the sense strand and linked to the adjacent nucleotide via a 5′-5′ internucleotide linkage, the targeting moiety can be attached at the 3′-position of the inverted abasic nucleotide or inverted deoxyribonucleotide. In other embodiments, the targeting moiety is covalently attached to the 3′ end of the sense strand. For example, in some embodiments, the targeting moiety is attached to the 3′-terminal nucleotide of the sense strand. In certain such embodiments, the targeting moiety is attached at the 3′-position of the 3′-terminal nucleotide of the sense strand. In embodiments in which an inverted abasic nucleotide or inverted deoxyribonucleotide is the 3′-terminal nucleotide of the sense strand and linked to the adjacent nucleotide via a 3′-3′ internucleotide linkage, the targeting moiety can be attached at the 5′-position of the inverted abasic nucleotide or inverted deoxyribonucleotide. In alternative embodiments, the targeting moiety is attached near the 3′ end of the sense strand, but before one or more terminal nucleotides (i.e. before 1, 2, 3, or 4 terminal nucleotides). In some embodiments, the targeting moiety is attached at the 2′-position of the sugar of the 3′-terminal nucleotide of the sense strand. In other embodiments, the targeting moiety is attached at the 2′-position of the sugar of the 5′-terminal nucleotide of the sense strand.

In certain embodiments, the targeting moiety is attached to the sense or antisense strand via a linker. A “linker” is an atom or group of atoms that covalently joins a ligand to a polynucleotide component of the LPA RNAi construct. The linker may be from about 1 to about 30 atoms in length, from about 2 to about 28 atoms in length, from about 3 to about 26 atoms in length, from about 4 to about 24 atoms in length, from about 6 to about 20 atoms in length, from about 7 to about 20 atoms in length, from about 8 to about 20 atoms in length, from about 8 to about 18 atoms in length, from about 10 to about 18 atoms in length, and from about 12 to about 18 atoms in length. In some embodiments, the linker may comprise a bifunctional linking moiety, which generally comprises an alkyl moiety with two functional groups. One of the functional groups is selected to bind to the compound of interest (e.g. sense or antisense strand of the RNAi construct) and the other is selected to bind essentially any selected group, such as a targeting moiety or component thereof as described herein. In certain embodiments, the linker comprises a chain structure or an oligomer of repeating units, such as ethylene glycol or amino acid units. Examples of functional groups that are typically employed in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Linkers that may be used to attach a targeting moiety to the sense or antisense strand in the LPA RNAi constructs used in the methods of the invention include, but are not limited to, pyrrolidine, 8-amino-3,6-dioxaoctanoic acid, succinimidyl 4-(N-maleimidomethyl)cyclohexane-1-carboxylate, 6-aminohexanoic acid, substituted C₁-C₁₀ alkyl, substituted or unsubstituted C₂-C₁₀ alkenyl or substituted or unsubstituted C₂-C₁₀ alkynyl. Preferred substituent groups for such linkers include, but are not limited to, hydroxyl, amino, alkoxy, carboxy, benzyl, phenyl, nitro, thiol, thioalkoxy, halogen, alkyl, aryl, alkenyl and alkynyl. Other types of linkers suitable for attaching targeting moieties to the sense or antisense strands in the LPA RNAi constructs sued in the methods of the invention are known in the art and can include the linkers described in U.S. Pat. Nos. 7,723,509; 8,017,762; 8,828,956; 8,877,917; and 9,181,551.

In certain embodiments, the targeting moiety covalently attached to the sense or antisense strand of the LPA RNAi constructs used in the methods of the invention comprises a GalNAc moiety, e.g, a multivalent GalNAc moiety. In some embodiments, the multivalent GalNAc moiety is a trivalent GalNAc moiety and is attached to the 3′ end of the sense strand. In other embodiments, the multivalent GalNAc moiety is a trivalent GalNAc moiety and is attached to the 5′ end of the sense strand. In yet other embodiments, the multivalent GalNAc moiety is a tetravalent GalNAc moiety and is attached to the 3′ end of the sense strand. In still other embodiments, the multivalent GalNAc moiety is a tetravalent GalNAc moiety and is attached to the 5′ end of the sense strand.

In certain embodiments, the LPA RNAi constructs used in the methods of the invention comprise a targeting moiety having the following structure [Structure 1]:

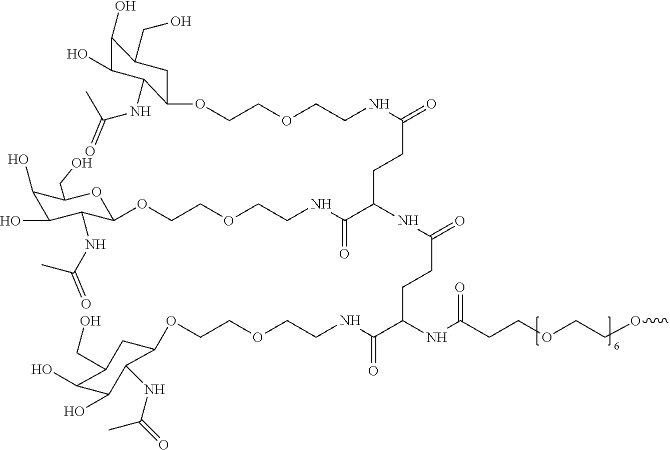

In preferred embodiments, the targeting moiety having this structure is covalently attached to the 5′ end of the sense strand via a phosphorothioate or phosphodiester bond.

In certain embodiments, the LPA RNAi construct suitable for use in the methods of the invention comprises:
a sense strand and an antisense strand, each of which is about 19 to about 23 nucleotides in length, wherein the antisense strand comprises a sequence that is complementary to an LPA mRNA sequence and the sense strand comprises a sequence that is complementary to the sequence of the antisense strand; and
a targeting moiety comprising an asialoglycoprotein receptor ligand, wherein the targeting moiety is covalently attached to the 5′ end of the sense strand. In some embodiments, the LPA RNAi construct has two blunt ends. For instance, in some such embodiments, the sense strand and antisense strand are each 21 nucleotides in length and hybridize to each other to form a duplex region that is 21 base pairs in length. In other such embodiments, the sense strand and antisense strand are each 19 nucleotides in length and hybridize to each other to form a duplex region that is 19 base pairs in length. In other embodiments, the LPA RNAi construct has two nucleotide overhangs. In one such embodiment, the LPA RNAi construct comprises (i) a sense strand and an antisense strand that are each 21 nucleotides in length, (ii) a duplex region that is 19 base pairs in length, and (iii) nucleotide overhangs of 2 unpaired nucleotides at both the 3′ end of the sense strand and the 3′ end of the antisense strand.

In some embodiments, the targeting moiety comprises a trivalent GalNAc moiety, such as any of the trivalent GalNAc moieties described in U.S. Pat. No. 10,246,709, which is hereby incorporated by reference in its entirety. In one preferred embodiment, the target moiety has the structure of Structure 1 described above.

In certain embodiments, the antisense strand of the LPA RNAi construct comprises a sequence that is substantially complementary or fully complementary to nucleotides 2706 to 2726 of the human LPA mRNA transcript set forth in NCBI Reference sequence NM 005577.4, nucleotides 2697 to 2726 of the human LPA mRNA transcript set forth in NCBI Reference sequence NM 005577.4, or nucleotides 2708 to 2725 of the human LPA mRNA transcript set forth in NCBI Reference sequence NM 005577.4. In such embodiments, the LPA RNAi construct may comprise a sense strand that is substantially complementary or fully complementary to the antisense strand targeting this region. Thus, in these embodiments, the sense strand may comprise a sequence identical to nucleotides 2706 to 2726, nucleotides 2697 to 2726, or nucleotides 2708 to 2725 of the human LPA mRNA transcript set forth in NCBI Reference sequence NM 005577.4.

In some embodiments, the sense strand of the LPA RNAi construct used in the methods of the invention comprises the sequence of 5′-GCCCCUUAUUGUUAUACG-3′ (SEQ ID NO: 1). In related embodiments, the antisense strand of the LPA RNAi construct used in the methods of the invention comprises the sequence of 5′-CGUAUAACAAUAAGGGGC-3′ (SEQ ID NO: 2).

Examples of LPA RNAi constructs suitable for use in the methods of the invention are described in WO 2017/059223, which is hereby incorporated by reference in its entirety. Duplexes AD03851, AD03853, and AD03536 described in WO 2017/059223 are particularly useful in the methods of the invention. In certain preferred embodiments, the LPA RNAi construct used in the methods of the invention comprises a sense strand comprising or consisting of the sequence of 5′-CAGCCCCUUAUUGUUAUACGA-3′ (SEQ ID NO: 3) and an antisense strand comprising or consisting of the sequence of 5′-UCGUAUAACAAUAAGGGGCUG-3′ (SEQ ID NO: 4). In related embodiments, the LPA RNAi construct used in the methods of the invention comprises a sense strand comprising or consisting of the sequence of modified nucleotides according to the sequence of 5′-csagccccuUfAfUfuguuauacgs(invdA)-3′ (SEQ ID NO: 5) and an antisense strand comprising or consisting of the sequence of modified nucleotides according to the sequence of 5′-usCfsgUfaUfaacaaUfaAfgGfgGfcsUfsg-3′ (SEQ ID NO: 6), wherein a, g, c, and u are 2′-O-methyl adenosine, 2′-O-methyl guanosine, 2′-O-methyl cytidine, and 2′-O-methyl uridine, respectively; Af, Gf, Cf, and Uf are 2′-deoxy-2′-fluoro (“2′-fluoro”) adenosine, 2′-fluoro guanosine, 2′-fluoro cytidine, and 2′-fluoro uridine, respectively; invdA is an inverted deoxyadenosine (3′-3′ linked nucleotide), and s is a phosphorothioate linkage. In some such embodiments, a targeting moiety having the structure of Structure 1 described herein is covalently attached to the 5′ end of the sense strand via a phosphorothioate linkage.

In other embodiments, the LPA RNAi construct used in the methods of the invention comprises a sense strand comprising the sequence of 5′-GCCCCUUAUUGUUAUACGAUU-3′ (SEQ ID NO: 7) and an antisense strand comprising the sequence of 5′-UCGUAUAACAAUAAGGGGCUU-3′ (SEQ ID NO: 8). In related embodiments, the LPA RNAi construct used in the methods of the invention comprises a sense strand comprising or consisting of the sequence of modified nucleotides according to the sequence of 5′-gsccccuUfAfUfuguuauacgauus(invAb)-3′ (SEQ ID NO: 9) and an antisense strand comprising or consisting of the sequence of modified nucleotides according to the sequence of 5′-usCfsgUfaUfaacaaUfaAfgGfgGfcsusu-3′ (SEQ ID NO: 10), wherein a, g, c, and u are 2′-O-methyl adenosine, 2′-O-methyl guanosine, 2′-O-methyl cytidine, and 2′-O-methyl uridine, respectively; Af, Gf, Cf, and Uf are 2′-deoxy-2′-fluoro (“2′-fluoro”) adenosine, 2′-fluoro guanosine, 2′-fluoro cytidine, and 2′-fluoro uridine, respectively; invAb is an inverted abasic nucleotide (3′-3′ linked nucleotide), and s is a phosphorothioate linkage. In some such embodiments, a targeting moiety having the structure of Structure 1 described herein is covalently attached to the 5′ end of the sense strand via a phosphorothioate linkage. In other related embodiments, the LPA RNAi construct used in the methods of the invention comprises a sense strand comprising or consisting of the sequence of modified nucleotides according to the sequence of 5′-(invAb)GfcCfcCfuUfAfUfuGfuUfaUfaCfgausu(invAb)-3′ (SEQ ID NO: 11) and an antisense strand comprising or consisting of the sequence of modified nucleotides according to the sequence of 5′-usCfsgsUfaUfaAfCfAfauaAfgGfgGfcusu-3′ (SEQ ID NO: 12), wherein a, g, c, and u are 2′-O-methyl adenosine, 2′-O-methyl guanosine, 2′-O-methyl cytidine, and 2′-O-methyl uridine, respectively; Af, Gf, Cf, and Uf are 2′-deoxy-2′-fluoro (“2′-fluoro”) adenosine, 2′-fluoro guanosine, 2′-fluoro cytidine, and 2′-fluoro uridine, respectively; invAb is an inverted abasic nucleotide (5′-5′ linked nucleotide when on the 5′ end of the strand and 3′-3′ linked nucleotide when on the 3′ end of the strand), and s is a phosphorothioate linkage. In some of these embodiments, a targeting moiety having the structure of Structure 1 described herein is covalently attached to the 5′ end of the sense strand via a phosphodiester linkage.

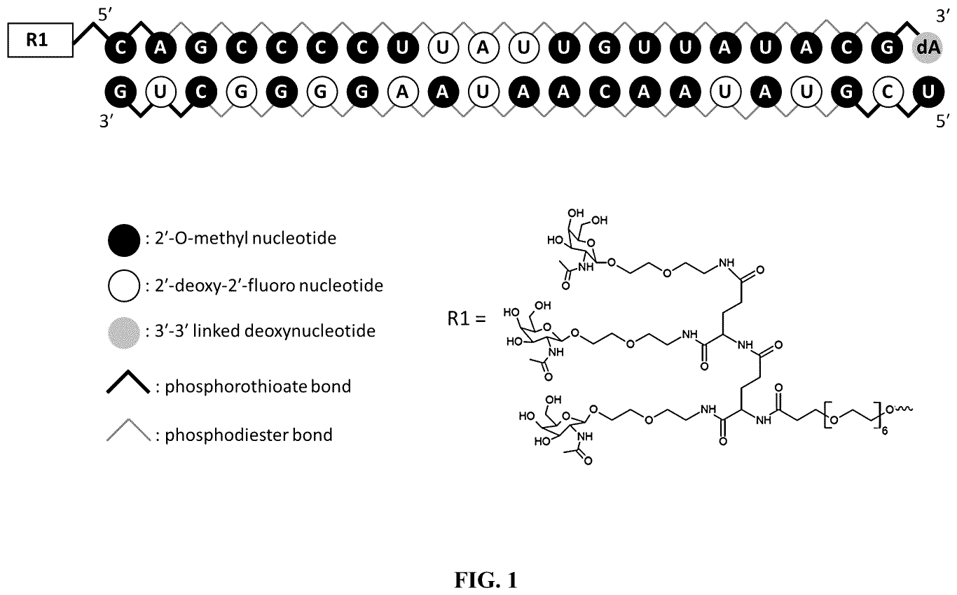

In certain preferred embodiments, the LPA RNAi construct administered to a patient according to the methods of the invention is olpasiran. The structure of olpasiran is shown schematically in FIG. 1 and is also described in WO 2017/059223, in which olpasiran is denoted as duplex no. AD03851. Olpasiran is a double-stranded siRNA molecule comprising two separate strands—a sense strand and an antisense strand, each of which is 21 nucleotides in length. The nucleobase sequences of the sense strand and antisense strand are fully complementary to each other and hybridize to form a duplex of 21 base pairs in length. The nucleotide sequences for the sense strand and antisense strand of olpasiran are set forth in SEQ ID NO: 3 and SEQ ID NO: 4, respectively. Both the sense strand and antisense strand of olpasiran are comprised of modified nucleotides and the modified sequences for each strand are set forth in SEQ ID NO: 5 (sense strand) and SEQ ID NO: 6 (antisense strand). A trivalent GalNAc moiety having the structure of Structure 1 (and represented as R1 in FIG. 1) is covalently attached to the 5′ end of the sense strand of olpasiran by a phosphorothioate linkage. The term olpasiran refers to the free acid of the compound shown in FIG. 1 as well as pharmaceutically acceptable salts thereof, such as a sodium salt.

The LPA RNAi constructs for use in the methods of the invention can readily be made using techniques known in the art, for example, using conventional nucleic acid solid phase synthesis. The polynucleotides of the RNAi constructs can be assembled on a suitable nucleic acid synthesizer utilizing standard nucleotide or nucleoside precursors (e.g. phosphoramidites). Automated nucleic acid synthesizers are sold commercially by several vendors, including DNA/RNA synthesizers from Applied Biosystems (Foster City, CA), MerMade synthesizers from BioAutomation (Irving, TX), and OligoPilot synthesizers from GE Healthcare Life Sciences (Pittsburgh, PA). Exemplary methods for synthesizing the LPA RNAi constructs as well as select targeting moieties are described in the Examples of WO 2017/059223 and U.S. Pat. No. 10,246,709, both of which are hereby incorporated by reference in their entireties.

A 2′ silyl protecting group can be used in conjunction with acid labile dimethoxytrityl (DMT) at the 5′ position of ribonucleosides to synthesize oligonucleotides via phosphoramidite chemistry. Final deprotection conditions are known not to significantly degrade RNA products. All syntheses can be conducted in any automated or manual synthesizer on large, medium, or small scale. The syntheses may also be carried out in multiple well plates, columns, or glass slides.

The 2′-O-silyl group can be removed via exposure to fluoride ions, which can include any source of fluoride ion, e.g., those salts containing fluoride ion paired with inorganic counterions e.g., cesium fluoride and potassium fluoride or those salts containing fluoride ion paired with an organic counterion, e.g., a tetraalkylammonium fluoride. A crown ether catalyst can be utilized in combination with the inorganic fluoride in the deprotection reaction. Preferred fluoride ion sources are tetrabutylammonium fluoride or aminohydrofluorides (e.g., combining aqueous HF with triethylamine in a dipolar aprotic solvent, e.g., dimethylformamide).

The choice of protecting groups for use on the phosphite triesters and phosphotriesters can alter the stability of the triesters towards fluoride. Methyl protection of the phosphotriester or phosphitetriester can stabilize the linkage against fluoride ions and improve process yields.

Since ribonucleosides have a reactive 2′ hydroxyl substituent, it can be desirable to protect the reactive 2′ position in RNA with a protecting group that is orthogonal to a 5′-O-dimethoxytrityl protecting group, e.g., one stable to treatment with acid. Silyl protecting groups meet this criterion and can be readily removed in a final fluoride deprotection step that can result in minimal RNA degradation.

Tetrazole catalysts can be used in the standard phosphoramidite coupling reaction. Preferred catalysts include, e.g., tetrazole, S-ethyl-tetrazole, benzylthiotetrazole, p-nitrophenyltetrazole.

As can be appreciated by the skilled artisan, further methods of synthesizing the LPA RNAi constructs described herein will be evident to those of ordinary skill in the art. Additionally, the various synthetic steps may be performed in an alternate sequence or order to give the desired compounds. Other synthetic chemistry transformations, protecting groups (e.g., for hydroxyl, amino, etc. present on the bases) and protecting group methodologies (protection and deprotection) useful in synthesizing the RNAi constructs are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. Custom synthesis of RNAi agents is also available from several commercial vendors, including Agilent Technologies (Santa Clara, CA), Nitto Denko Avecia (Milford, MA), Dharmacon, Inc. (Lafayette, CO), AxoLabs GmbH (Kulmbach, Germany), and Ambion, Inc. (Foster City, CA).

The LPA RNAi construct is generally administered to a patient in a pharmaceutical composition, which can include pharmaceutically acceptable carriers, excipients, or diluents. Thus, the present invention also includes pharmaceutical compositions and formulations comprising the LPA RNAi constructs and pharmaceutically acceptable carriers, excipients, or diluents for use in the methods of the invention described herein. For clinical applications, pharmaceutical compositions and formulations will be prepared in a form appropriate for the intended application. Generally, this will entail preparing compositions that are essentially free of pyrogens, as well as other impurities that could be harmful to humans or animals.

The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable carrier, excipient, or diluent” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the LPA RNAi constructs described herein, its use in therapeutic compositions is contemplated. Supplementary active ingredients also can be incorporated into the compositions, provided they do not inactivate the LPA RNAi constructs of the compositions.

Compositions and methods for the formulation of pharmaceutical compositions depend on a number of criteria, including, but not limited to, route of administration, type and extent of disease or disorder to be treated, or dose to be administered. In some embodiments, the pharmaceutical compositions are formulated based on the intended route of delivery. For instance, in certain embodiments, the pharmaceutical compositions are formulated for parenteral delivery. Parenteral forms of delivery include intravenous, intraarterial, subcutaneous, intrathecal, intraperitoneal or intramuscular injection or infusion. In one embodiment, the pharmaceutical composition is formulated for intravenous delivery. In another embodiment, the pharmaceutical composition is formulated for subcutaneous delivery. In some embodiments, the pharmaceutical compositions comprise an effective amount of an LPA RNAi construct. An effective amount of an LPA RNAi construct, particularly olpasiran, may be any of the doses described herein.

Administration of the pharmaceutical compositions comprising the LPA RNAi construct according to the methods of the present invention may be via any common route so long as the target tissue is available via that route. Such routes include, but are not limited to, parenteral (e.g., subcutaneous, intramuscular, intraperitoneal or intravenous), oral, nasal, buccal, intradermal, transdermal, and sublingual routes, or by direct injection into liver tissue or delivery through the hepatic portal vein. In some embodiments of the methods of the invention, the LPA RNAi construct or a pharmaceutical composition comprising the LPA RNAi construct is administered to a patient parenterally. For instance, in certain embodiments, the LPA RNAi construct or pharmaceutical composition comprising the LPA RNAi construct is administered intravenously. In other embodiments, the LPA RNAi construct or pharmaceutical composition comprising the LPA RNAi pharmaceutical composition is administered subcutaneously, for example, by subcutaneous injection. In such embodiments, the subcutaneous injection volume is about 2 mL or less, for example, about 2 mL, about 1.8 mL, about 1.7 mL, about 1.6 mL, about 1.5 mL, about 1.4 mL, about 1.3 mL, about 1.2 mL, about 1.1 mL, about 1 mL, about 0.9 mL, about 0.8 mL, about 0.7 mL, about 0.6 mL, or about 0.5 mL. In one embodiment, the subcutaneous injection volume is about 1 mL or less. In another embodiment, the subcutaneous injection volume is about 1 mL. In yet another embodiment, the subcutaneous injection volume is about 1.5 mL.

In embodiments in which the pharmaceutical composition is administered by parenteral injection, the pharmaceutical composition can be administered to the patient with a syringe. In some embodiments, the syringe is pre-filled with the pharmaceutical composition. In other embodiments in which the pharmaceutical composition is administered to the patient by parenteral injection, such as subcutaneous injection, the pharmaceutical composition is administered with an injection device, including devices for self-administration. Such devices are commercially available and include, but are not limited to, autoinjectors, dosing pens, microinfusion pumps, on-body injectors, and pre-filled syringes. Exemplary devices for administering a pharmaceutical composition comprising an effective amount of an LPA RNAi construct (e.g. olpasiran) according to the methods of the invention include autoinjectors (e.g., SureClick®, EverGentle®, Avanti®, DosePro®, Molly®, and Leva®), pen injection devices (e.g., Madie® pen injector, DCP™ pen injector, BD Vystra™ disposable pen, BD™ reusable pen), and pre-filled syringes (BD Sterifill™, BD Hypak™, prefilled syringes from Baxter). In some embodiments, the pharmaceutical composition comprising an effective amount of an LPA RNAi construct (e.g. olpasiran) is administered to the patient with a pre-filled syringe. In other embodiments, the pharmaceutical composition comprising an effective amount of an LPA RNAi construct (e.g. olpasiran) is administered to the patient with an autoinjector. In certain such embodiments, the injection volume of the syringe, autoinjector, or other injection device is about 2 mL or less, for example, about 2 mL, about 1.8 mL, about 1.7 mL, about 1.6 mL, about 1.5 mL, about 1.4 mL, about 1.3 mL, about 1.2 mL, about 1.1 mL, about 1 mL, about 0.9 mL, about 0.8 mL, about 0.7 mL, about 0.6 mL, or about 0.5 mL. In one embodiment, the injection volume of the syringe, autoinjector, or other injection device is about 1 mL or less. In another embodiment, the injection volume of the syringe, autoinjector, or other injection device is about 1 mL. In yet another embodiment, the injection volume of the syringe, autoinjector, or other injection device is about 1.5 mL.

Colloidal dispersion systems, such as macromolecule complexes, nanocapsules, microspheres, beads, and lipid-based systems, including oil-in-water emulsions, micelles, mixed micelles, and liposomes, may be used as delivery vehicles for the LPA RNAi constructs of the invention. Commercially available fat emulsions that are suitable for delivering the nucleic acids of the invention include Intralipid® (Baxter International Inc.), Liposyn® (Abbott Pharmaceuticals), Liposyn® II (Hospira), Liposyn® III (Hospira), Nutrilipid (B. Braun Medical Inc.), and other similar lipid emulsions. A preferred colloidal system for use as a delivery vehicle in vivo is a liposome (i.e., an artificial membrane vesicle). The LPA RNAi constructs may be encapsulated within liposomes or may form complexes thereto, in particular to cationic liposomes. Alternatively, LPA RNAi constructs may be complexed to lipids, in particular to cationic lipids. Suitable lipids and liposomes include neutral (e.g., dioleoylphosphatidyl ethanolamine (DOPE), dimyristoylphosphatidyl choline (DMPC), and dipalmitoyl phosphatidylcholine (DPPC)), distearolyphosphatidyl choline), negative (e.g., dimyristoylphosphatidyl glycerol (DMPG)), and cationic (e.g., dioleoyltetramethylaminopropyl (DOTAP) and dioleoylphosphatidyl ethanolamine (DOTMA)). The preparation and use of such colloidal dispersion systems are well known in the art. Exemplary formulations are also disclosed in U.S. Pat. Nos. 5,981,505; 6,217,900; 6,383,512; 5,783,565; 7,202,227; 6,379,965; 6,127,170; 5,837,533; 6,747,014; and WO03/093449.

In some embodiments, the LPA RNAi constructs are fully encapsulated in a lipid formulation, e.g., to form a SNALP or other nucleic acid-lipid particle. As used herein, the term “SNALP” refers to a stable nucleic acid-lipid particle. SNALPs typically contain a cationic lipid, a non-cationic lipid, and a lipid that prevents aggregation of the particle (e.g., a PEG-lipid conjugate). SNALPs are exceptionally useful for systemic applications, as they exhibit extended circulation lifetimes following intravenous injection and accumulate at distal sites (e.g., sites physically separated from the administration site). The nucleic acid-lipid particles typically have a mean diameter of about 50 nm to about 150 nm, about 60 nm to about 130 nm, about 70 nm to about 110 nm, or about 70 nm to about 90 nm, and are substantially nontoxic. In addition, the nucleic acids when present in the nucleic acid-lipid particles are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO 96/40964.

The pharmaceutical compositions comprising an LPA RNAi construct suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions for use in the methods of the present invention generally may be formulated in a neutral or salt form. Pharmaceutically-acceptable salts include, for example, acid addition salts (formed with free amino groups) derived from inorganic acids (e.g., hydrochloric or phosphoric acids), or from organic acids (e.g., acetic, oxalic, tartaric, mandelic, and the like). Salts formed with the free carboxyl groups can also be derived from inorganic bases (e.g., sodium, potassium, ammonium, calcium, or ferric hydroxides) or from organic bases (e.g., isopropylamine, trimethylamine, histidine, procaine and the like). Sodium salts of the LPA RNAi constructs are particularly useful for therapeutic administration to human subjects. Thus, in certain preferred embodiments, the LPA RNAi constructs, particularly olpasiran, are in the form of a sodium salt. In other embodiments, the LPA RNAi constructs (e.g. olpasiran) are in the form of a potassium salt.

For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion or injection, (see for example, “Remington's Pharmaceutical Sciences” 15th Edition, pages 1035-1038 and 1570-1580). For human administration, preparations should meet sterility, pyrogenicity, general safety and purity standards as required by FDA standards. In certain embodiments, a pharmaceutical composition for use in the methods of the invention comprises or consists of a sterile saline solution and an LPA RNAi construct (e.g. olpasiran) described herein. In other embodiments, a pharmaceutical composition for use in the methods of the invention comprises or consists of an LPA RNAi construct (e.g. olpasiran) described herein and sterile water (e.g. water for injection, WFI). In still other embodiments, a pharmaceutical composition for use in the methods of the invention comprises or consists of an LPA RNAi construct (e.g. olpasiran) described herein and phosphate-buffered saline (PBS).

In certain embodiments, a pharmaceutical composition useful for treating, ameliorating, preventing, or reducing the risk of cardiovascular disease according to the methods of the invention comprises an effective amount of an LPA RNAi construct (e.g. olpasiran), potassium phosphate buffer, and sodium chloride. In some such embodiments, the pharmaceutical composition comprises about 10 mg/mL to about 200 mg/mL of an LPA RNAi construct (e.g. olpasiran), about 5 mM to about 30 mM potassium phosphate, and about 20 mM to about 160 mM sodium chloride. In other embodiments, the pharmaceutical composition comprises about 65 mg/mL to about 85 mg/mL of an LPA RNAi construct (e.g. olpasiran), about 15 mM to about 25 mM potassium phosphate, and about 70 mM to about 90 mM sodium chloride. In still other embodiments, the pharmaceutical composition comprises about 140 mg/mL to about 160 mg/mL of an LPA RNAi construct (e.g. olpasiran), about 15 mM to about 25 mM potassium phosphate, and about 30 mM to about 50 mM sodium chloride. The pH of any of these pharmaceutical compositions can be in the range of about 6.4 to about 7.2 (e.g., pH of about 6.4, about 6.6, about 6.8, about 7.0, or about 7.2).

In some embodiments, a pharmaceutical composition to be administered according to the methods of the invention comprises about 10 mg/mL of an LPA RNAi construct (e.g. olpasiran), about 5 mM to about 15 mM potassium phosphate, and about 135 mM to about 155 mM sodium chloride at a pH of 6.8±0.2. In one embodiment, the pharmaceutical composition comprises about 10 mg/mL of an LPA RNAi construct (e.g. olpasiran), about 10 mM potassium phosphate, and about 145 mM sodium chloride at a pH of 6.8. In other embodiments, a pharmaceutical composition to be administered according to the methods of the invention comprises about 75 mg/mL of an LPA RNAi construct (e.g. olpasiran), about 15 mM to about 25 mM potassium phosphate, and about 70 mM to about 90 mM sodium chloride at a pH of 6.8±0.2. In one such embodiment, the pharmaceutical composition comprises about 75 mg/mL of an LPA RNAi construct (e.g. olpasiran), about 20 mM potassium phosphate, and about 80 mM sodium chloride at a pH of 6.8. In certain embodiments, a pharmaceutical composition to be administered according to the methods of the invention comprises about 150 mg/mL of an LPA RNAi construct (e.g. olpasiran), about 15 mM to about 25 mM potassium phosphate, and about 30 mM to about 50 mM sodium chloride at a pH of 6.8±0.2. In one particular embodiment, the pharmaceutical composition comprises about 150 mg/mL of an LPA RNAi construct (e.g. olpasiran), about 20 mM potassium phosphate, and about 40 mM sodium chloride at a pH of 6.8.

Any of the LPA RNAi constructs described herein can be incorporated into any of the pharmaceutical compositions described above and administered to a patient according to the methods of the invention. In certain embodiments, the LPA RNAi construct incorporated into any of the pharmaceutical compositions described above and administered to a patient according to the methods of the invention is olpasiran.

In some embodiments, the pharmaceutical compositions of the invention are packaged with or stored within a device for administration, such as any of the injection devices described above (e.g. pre-filled syringes, autoinjectors, injection pumps, on-body injectors, and injection pens). Devices for aerosolized or powder formulations include, but are not limited to, inhalers, insufflators, aspirators, and the like. Thus, the present invention includes administration devices comprising a pharmaceutical composition described herein for treating or preventing one or more of the diseases or disorders described herein.

The following examples, including the experiments conducted and the results achieved, are provided for illustrative purposes only and are not to be construed as limiting the scope of the appended claims.

### EXAMPLES

### Example 1. A Phase 1, Randomized, Double-Blind, Placebo-Controlled, Single Ascending Dose Study to Evaluate the Safety, Tolerability, Pharmacokinetics and Pharmacodynamics of Olpasiran in Subjects with Elevated Plasma Lipoprotein(a)

Mendelian and epidemiological randomization studies have recently established lipoprotein(a) (Lp(a)) as a strong causal risk factor for myocardial infarction and other atherosclerotic complications. There are currently no approved medicines that selectively target Lp(a) and have demonstrated reduction in cardiovascular events. Olpasiran (also known as AMG 890) is an siRNA designed to reduce production of Lp(a) by targeting mRNA transcribed from the LPA gene. The structure of olpasiran is shown in FIG. 1.

This phase 1 study was a randomized, double-blind, placebo-controlled, single-ascending-dose study in subjects with elevated plasma Lp(a) conducted at 8 sites in the United States and Australia. Approximately 80 subjects were planned for enrollment in 9 single ascending dose cohorts; in each cohort, subjects were randomized 3:1 to receive olpasiran or placebo.

Eligible subjects were women of non-reproductive potential and men, both with age between 18 and 60 years, inclusive, for cohorts 1 to 5; and with age between 18 and 65 years, inclusive, for cohorts 6 to 9. For cohorts 1 to 5, plasma Lp(a) concentrations were ≥70 nmol/L and ≤199 nmol/L at screening; for cohorts 6 to 9, plasma Lp(a) concentrations were ≥200 nmol/L at screening; for cohorts 6 to 9, at least 6 subjects in each cohort were on a stable dose of statin for at least 6 weeks at the time of enrollment. Subjects were without any clinically significant abnormality in medical history at the time of randomization.

After providing informed consent, subjects were screened for eligibility over 28 days and were admitted to the research facility on day −1. After completion of pre-dose procedures, subjects received their dose of study drug (olpasiran or placebo). Subjects in cohorts 1 to 5 stayed in the research facility from day −1 to day 4 and returned to the facility for assessments through the end of the study. Subjects received single subcutaneous doses of 3 mg, 9 mg, 30 mg, 75 mg, and 225 mg for cohorts 1 through 5, respectively; and 9 mg, 75 mg, 225 mg, and 675 mg for cohorts 6 through 9 respectively (see Table 1 below)).

TABLE 1
Dosing Cohorts in Single-Ascending Dose Study

| Cohort | Number of Subjects | Treatment |
|---|---|---|
| 1^{a} | 6 | Olpasiran 3 mg |
| | 2 | Placebo |
| 2^{a} | 6 | Olpasiran 9 mg |
| | 2 | Placebo |
| 3^{a} | 6 | Olpasiran 30 mg |
| | 2 | Placebo |
| 4^{a} | 6 | Olpasiran 75 mg |
| | 2 | Placebo |
| 5^{a} | 6 | Olpasiran 225 mg |
| | 2 | Placebo |
| 6^{b} | 9 | Olpasiran 9 mg |
| | 3 | Placebo |
| 7^{a} | 9 | Olpasiran 75 mg |
| | 3 | Placebo |
| 8^{b} | 6 | Olpasiran 225 mg |
| | 2 | Placebo |
| 9^{b} | 6 | Olpasiran 675 mg |
| | 2 | Placebo |

^{a}Subjects with screening plasma Lp(a) ≥70 nmol/L and ≤199 nmol/L
^{b}Subjects with screening plasma Lp(a) ≥200 nmol/L

For cohorts 1 to 5, and cohort 9, the first 2 enrolled subjects in each cohort were randomized to receive either olpasiran or placebo in a 1:1 ratio (sentinel pair) and were dosed in a blinded fashion on the same day at the same study site. If deemed safe by the investigator, and no less than 24 hours after sentinel pair dosing, the same dose was administered to the remaining cohort subjects. Enrollment into cohorts 1 to 5 was staggered. Subsequent cohorts were dosed after the dose regimen in the preceding cohort was found by the Dose Level Review Team (DLRT) to be safe and reasonably well tolerated based on available safety data through study day 15 for all subjects. Enrollment into cohorts 5 to 7 was initiated after the dose regimen in cohort 4 was found by the DLRT to be safe and reasonably well tolerated based on available safety data through study day 15. Subjects returned to the facility for an end-of-treatment assessment on day 113 for cohorts 1 and 2 and on day 225 for cohorts 3 to 7. Subjects were to return for follow-up until the Lp(a) concentration was at least 80% of baseline (approximately every 2 weeks for cohorts 1 and 2 and monthly for cohorts 3 through 7). Blood and urine samples were collected throughout the study for the assessment of olpasiran pharmacokinetics (PK) and pharmacodynamics (PD). Safety variables were also regularly assessed.

The primary endpoints were safety and tolerability as measured by treatment-emergent adverse events (TEAEs), safety laboratory analytes, vital signs, and electrocardiograms (ECGs). The secondary endpoints were olpasiran PK parameters including, but not limited to, maximum observed concentration (Cₘₐₓ), the time of maximum observed concentration (tₘₐₓ), and area under the concentration-time curve (AUC); and PD parameters including the change and percentage change in plasma Lp(a) levels at each scheduled visit. Baseline values for Lp(a) were defined as the mean of screening and day 1 predose. If for any reason only one value was available, then that value was used as baseline. Exploratory endpoints included percentage change in low-density lipoprotein cholesterol (LDL-C) and total apolipoprotein B (ApoB) at each scheduled visit.

64 subjects were enrolled in the study and administered olpasiran or placebo (Cohorts 1-olpasiran, n=30, doses: 3 mg, 9 mg, 30 mg, 75 mg, 225 mg; placebo, n=10; Cohorts 6-7 olpasiran, n=18, doses: 9 mg and 75 mg; placebo, n=6). For subjects administered olpasiran in cohorts 1 to 5 (n=30), subjects had a mean (SD) age of 43.9 (13.5) years, 30.0% were women, 63.3% were of Hispanic or Latino ethnicity, 30.0% were black or African American, and 70.0% were white. For subjects administered placebo in cohorts 1 to 5 (n=10), subjects had a mean (SD) age of 46.3 (8.5) years, 30.0% were women, 50.0% were of Hispanic or Latino ethnicity, were black or African American, and 70.0% were white. For subjects administered olpasiran in cohorts 6 and 7 (n=18), subjects had a mean (SD) age of 52.7 (9.4) years, 33.3% were women, 27.8% were of Hispanic or Latino ethnicity, and 88.9% were white. For subjects administered placebo in cohorts 6 and 7 (n=6), subjects had a mean (SD) age of 57.8 (5.8) years, 66.7% were women, 33.3% were of Hispanic or Latino ethnicity, and 83.3% were white. 67% of all subjects enrolled in cohorts 6 and 7 (n=24) had statin use at baseline. Subjects had few comorbidities. There was no use of lipid-regulating medications in cohorts 1 to 5, but a substantial proportion of subjects in cohorts 6 and 7 were on statins and/or ezetimibe. Median (Q1, Q3) baseline Lp(a) concentrations were 124 nmol/L (104, 137) in subjects receiving placebo in cohorts 1 to 5 and 122 nmol/L (97, 146) for subjects receiving olpasiran in cohorts 1 to 5. Median (Q1, Q3) baseline Lp(a) concentrations were 272 nmol/L (233, 307) in subjects receiving placebo in cohorts 6 and 7 and 253 nmol/L (224, 334) for subjects receiving olpasiran in cohorts 6 and 7.

Olpasiran appeared to be well tolerated. There were no treatment-related serious adverse events. One placebo subject had an adverse event of serious non-cardiac chest pain, which was deemed unrelated to treatment. In cohorts 1-5, the most common TEAE was upper respiratory infection (10% placebo, 13% olpasiran). See Table 2 below. In cohorts 6-7, the most common TEAEs were headache (50% placebo, 28% olpasiran) and upper respiratory infection (17% placebo, 17% olpasiran) (Table 2). Only one subject in the study experienced an injection site reaction. There was no apparent dose relationship with the frequency of adverse events. No clinically relevant changes in liver tests, platelets or coagulation parameters, or renal function were observed.

TABLE 2
Treatment-Emergent Adverse Events in Single-Ascending Dose Study

| | Cohorts 1-5 | | Cohorts 6-7 | |
|---|---|---|---|---|
| | Screening Lp(a) ≥70 and ≤199 nmol/L | | Screening Lp(a) ≥200 nmol/L | |
| Adverse Events | Placebo | Olpasiran | Placebo | Olpasiran |
| (AE), n (%) | (N = 10) | (N = 30) | (N = 6) | (N = 18) |
| Any AE | 5 (50.0) | 12 (40.0) | 4 (66.7) | 10 (55.6) |
| Serious AE | 0 | 0 | 1 (16.7) | 0 |

AEs occurring in more than one subject across cohorts

| | | | | |
|---|---|---|---|---|
| Headache | 1 (10.0) | 0 | 3 (50.0) | 5 (27.8) |
| Upper respiratory | 1 (10.0) | 4 (13.3) | 1 (16.7) | 3 (16.7) |
| tract infection | | | | |
| Viral upper | 0 | 1 (3.3) | 0 | 2 (11.1) |
| respiratory tract | | | | |
| infection | | | | |
| Non-cardiac chest | 1 (10.0) | 1 (3.3) | 1 (16.7) | 0 |
| pain | | | | |
| Blood creatine | 1 (10.0) | 1 (3.3) | 0 | 0 |
| phosphokinase | | | | |
| increased | | | | |
| Back pain | 1 (10.0) | 1 (3.3) | 0 | 3 (16.7) |
| Contusion | 0 | 1 (3.3) | 0 | 1 (5.6) |
| Skin abrasion | 0 | 1 (3.3) | 0 | 1 (5.6) |
| Fatigue | 0 | 0 | 1 (16.7) | 1 (5.6) |
| Arthralgia | 0 | 1 (3.3) | 0 | 1 (5.6) |
| Epistaxis | 1 (10.0) | 1 (3.3) | 0 | 0 |

AEs of special interest

| | | | | |
|---|---|---|---|---|
| Injection site | 0 | 1 (3.3) | 0 | 0 |
| reaction | | | | |

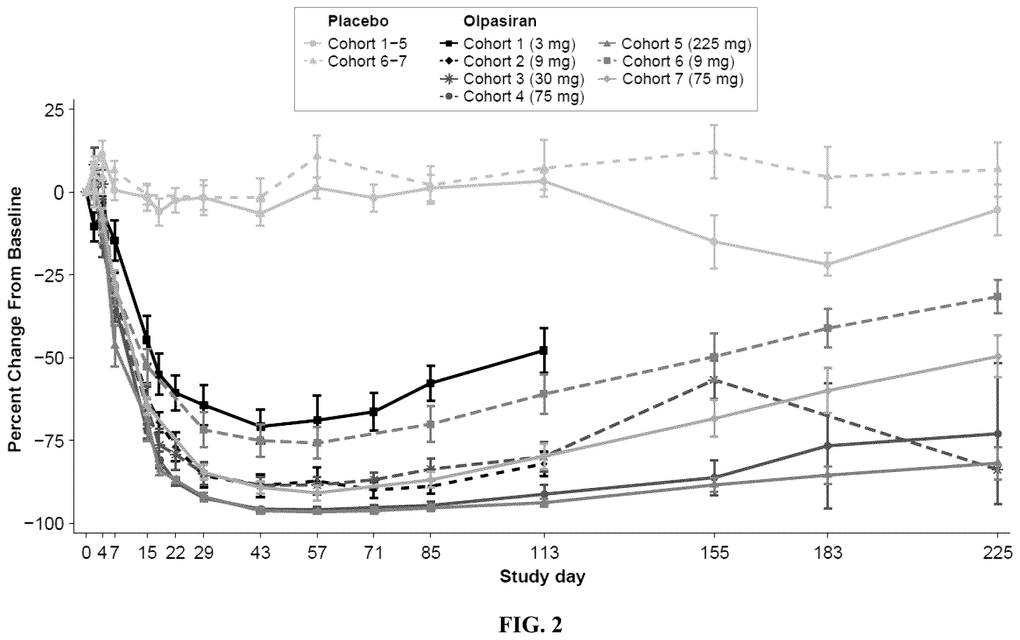

Lp(a) suppression occurred in a dose-responsive manner. As shown in FIG. 2, in cohorts 1-5, single doses of olpasiran effectively reduced mean Lp(a) levels from baseline by 71-96% (based on doses) at Day 43, and by 80-94% at Day 113 (cohorts 2-5). In cohorts 6 and 7, single doses of olpasiran effectively lowered mean Lp(a) levels from baseline by 75% and 89% at Day 43, respectively, and by 61% and 80% at Day 113, respectively (FIG. 2). A sharp decline in Lp(a) was observed from day 15, with maximum Lp(a) suppression observed between days 43 and 71. Lp(a) concentrations gradually recovered but remained well below placebo levels at day 225. Single doses of 9 mg or greater led to reductions in Lp(a) persisting for 3 to 6 months.

Pharmacokinetic parameters for olpasiran in each of the seven dosing cohorts are shown in Table 3 below. After single doses of 3, 9, 30, 75 and 225 mg (Cohorts 1 to 5), olpasiran was rapidly absorbed with geometric mean Cₘₐₓ occurring within 7.5 hours after dosing. Geometric mean half-life (t_{1/2}) values ranged from 3 to 8 hours with the vast majority of olpasiran cleared from the serum within 2-3 days. Systemic exposures increased in an approximately dose-proportional manner at doses up to 225 mg. Olpasiran AUC exposures in subjects with baseline Lp(a)≥200 nmol/L (Cohorts 6 and 7) were approximately 18-33% lower than in subjects with baseline Lp(a)≥70 to ≤199 nmol/L (Cohorts 2 and 4).

TABLE 3
Olpasiran Pharmacokinetic Parameters in Single-Ascending Dose Study

| | | tₘₐₓ | Cₘₐₓ | DN-Cₘₐₓ | AUC_{inf} | DN-AUC_{inf} | *t_{1/2, z} |
|---|---|---|---|---|---|---|---|
| Dose | N | (hr) | (ng/mL) | (ng/mL/mg) | (hr · ng/mL) | (hr · ng/mL/mg) | (hr) |
| 3 mg | 6 | 4.5 | 11.7 | 3.89 | 172^{a} | 57.3^{a} | 7.72^{a} |
| (Cohort 1) | | (1.0-1340) | (11.8, 19%) | (3.95, 19%) | (172, NR %) | (57.3, NR %) | (10.2, NR %) |
| 9 mg | 6 | 3.0 | 32.7 | 3.63 | 408^{b} | 45.3^{b} | 2.83^{b} |
| (Cohort 2) | | (3.0-6.0) | (36.1, 55%) | (4.01, 55%) | (427, 34%) | (47.5, 34%) | (2.85, 16%) |
| 9 mg | 9 | 3.0 | 15.2 | 1.69 | 272^{c} | 30.2^{c} | 5.31^{c} |
| (Cohort 6) | | (1.0-9.0) | (16.8, 41%) | (1.87, 41%) | (285, 28%) | (31.6, 28%) | (5.71, 46%) |
| 30 mg | 6 | 7.5 | 71.6 | 2.39 | 1030^{e} | 33.4^{e} | 3.44^{e} |
| (Cohort 3) | | (1.0-9.0) | (74.9, 33%) | (2.50, 33%) | (1070, 31%) | (35.7, 31%) | (3.53, 24%) |
| 75 mg | 6 | 4.5 | 218 | 2.91 | 2500^{d} | 33.3^{d} | 3.63^{d} |
| (Cohort 4) | | (0.17-9.0) | (252, 61%) | (3.36, 61%) | (2520, 17%) | (33.6, 17%) | (3.68, 21%) |
| 75 mg | 9 | 6.0 | 97.7 | 1.30 | 2040^{b} | 27.2^{b} | 5.72^{b} |
| (Cohort 7) | | (3.0-24) | (111, 51%) | (1.48, 51%) | (2050, 11%) | (27.4, 11%) | (6.22, 43%) |
| 225 mg | 6 | 6.0 | 385 | 1.71 | 9380^{b} | 41.7^{b} | 6.69 |
| (Cohort 5) | | (1.0-12) | (421, 53%) | (1.87, 53%) | (9600, 27%) | (42.7, 27%) | (6.72, 11%) |

Data presented as geometric mean (mean, CV %) for all pharmacokinetic parameters except for tₘₐₓ, which is presented as median (range). Values are reported to 3 significant figures except for tₘₐₓ and CV % which are presented as two significant figures and one decimal place, respectively.
*t_{1/2, z} values for the 225 mg dose group represent beta half-life; other dose groups report gamma half-life.
^{a}N = 2;
^{b}N = 4;
^{c}N = 6;
^{d}N = 3;
^{e}N = 5
t_{max,} time to reach Cₘₐₓ;
C_{max,} maximum observed drug concentration;
DN, dose-normalized;
AUC_{inf,} area under the plasma concentration-time curve from time zero to infinity;
t_{1/2, z,} terminal half-life

The results of the phase 1 study demonstrate that in adults with elevated Lp(a), single-dose treatment of olpasiran was well-tolerated and significantly reduced Lp(a) with observed approximate median percent reductions of >90% and effects persisting for 3 to 6 months at doses of 9 mg or higher. The 75 and 225 mg doses in the high Lp(a) group (Lp(a)≥70 to ≤199 nmol/L) were nearly superimposable with respect to effects on Lp(a) concentration; similarly, the 9 and 30 mg doses were nearly superimposable. However, the 9 and 75 mg doses in the very high Lp(a) group (Lp(a)≥200 nmol/L) showed reduced percent suppression of Lp(a) from baseline relative to the same doses in the high Lp(a) cohorts.

The depth and duration of suppression of Lp(a) levels observed with these single, low doses of olpasiran were significantly better than expected from the projected human doses based on studies of olpasiran in cynomolgus monkeys. Based on efficacy data for olpasiran in cynomolgus monkeys (see, e.g., Example 18 in WO 2017/059223), a projected human dose of 75 mg was predicted to reduce Lp(a) levels by about 80% for at least one month. Remarkably, as described above, single doses as low as 9 mg of olpasiran reduced Lp(a) levels in human subjects by greater than 80% for greater than 3 months. Single olpasiran doses of 75 mg and 225 mg suppressed Lp(a) levels by greater than 80% for more than six months. Thus, olpasiran can be administered to human patients in need of reduction of Lp(a) at lower doses and longer dosing intervals, including up to once every 6 months. Such dosing regimens have a number of different benefits, such as improved patient adherence, reduced cost of medication, and reduced volume and number of injections.

### Example 2. Olpasiran PK/PD Model for Design of Dosing Regimens for Optimal Lp(a) Reduction

A mathematical model was developed to characterize olpasiran pharmacokinetics and Lp(a) suppression in healthy volunteers with elevated plasma Lp(a) (≥70 nmol/L to <200 nmol/L for Cohorts 1-5, ≥200 nmol/L for Cohorts 6-7) based on the phase 1 data described in Example 1. The pharmacokinetics (PK) of olpasiran was described using a PK model with first order absorption from the subcutaneous administration site to circulation, distribution to the liver via asialoglycoprotein receptor (ASGPR) uptake, recycling of olpasiran back to circulation from the liver, and elimination via degradation from systemic circulation and the liver. Olpasiran serum exposure was found to correlate with baseline Lp(a). Thus, a function to modulate olpasiran bioavailability by baseline Lp(a) was also included in the model. Suppression of Lp(a) from baseline was described using a PK/PD model, whereby the model-predicted olpasiran liver concentrations accelerated the degradation of LPA mRNA leading to reduced production and suppression of Lp(a) for the duration of sufficient olpasiran concentrations in the liver. The relationship between olpasiran liver concentration and LPA mRNA degradation was modeled using an Eₘₐₓ model. Changes in LPA mRNA concentrations were inferred based on degree of Lp(a) suppression. Synthesis and degradation rates of Lp(a) were informed by baseline Lp(a) levels, with higher baseline values associated with greater production rates.

The following assumptions were made during model development and for clinical dosing regimen simulations:
a) simulations for Phase 2 dose selection were performed for a target population having ≥150 nmol/L baseline Lp(a) levels, however, model parameters were estimated from subjects with baseline Lp(a) values≥70 nmol/L to <200 nmol/L and >200 nmol/L;
b) between- and within-subject variability following multiple doses for the phase 2 population were assumed to be the same as that estimated for Phase 1 study subjects; and
c) duration of Lp(a) suppression is based on model predicted olpasiran PK/PD half-life in the liver and the extrapolation of response following multiple doses is based on observed suppression from the Phase 1 study and accumulation of effect at end of the dosing interval.

This model was able to adequately predict the observed significant decrease in olpasiran exposure in subjects with higher baseline Lp(a) (≥150 nmol/L), suggesting that higher doses of olpasiran may be necessary to achieve target Lp(a) suppression in this patient population. Simulations of this model were performed to explore Q3M and Q6M dosing regimens and extrapolate predicted Lp(a) suppression after multiple doses of olpasiran. For each proposed dosing regimen, the proportion of subjects achieving target Lp(a) suppression (≥80% reduction from baseline) and the proportion of subjects achieving absolute Lp(a) values≤50 nmol/L at the end of the dosing interval were calculated.

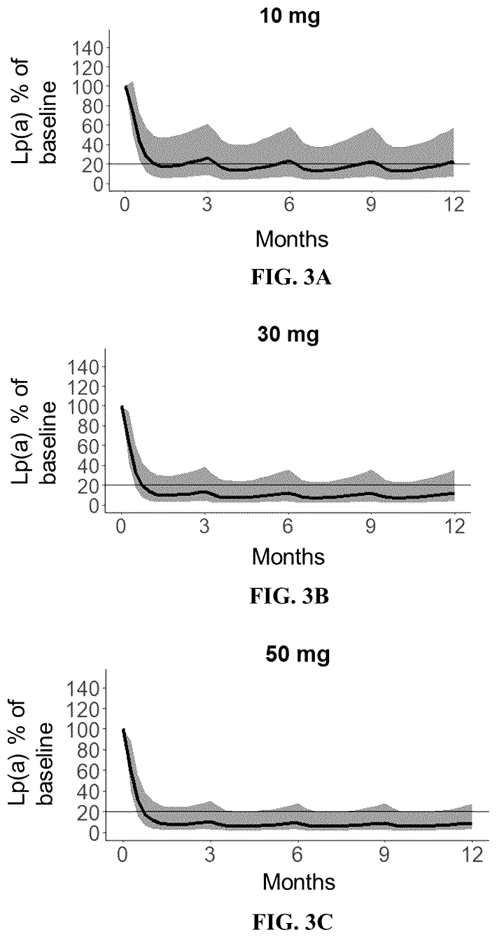

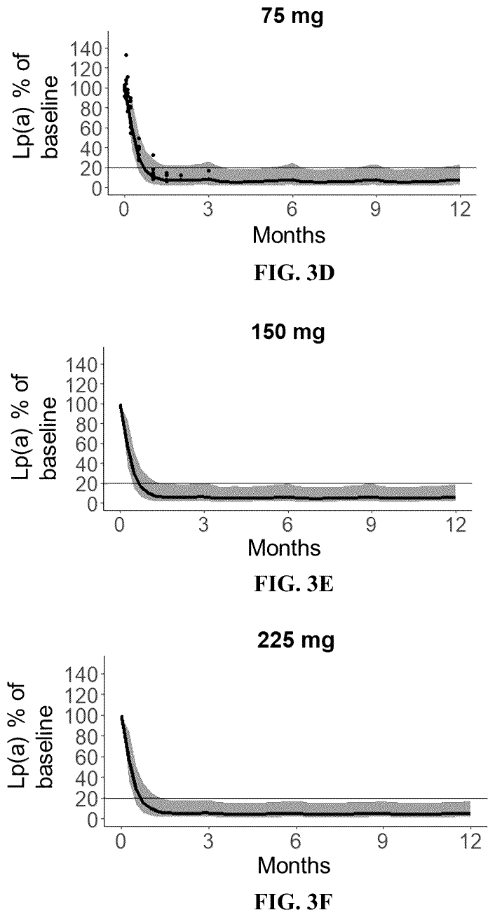

FIGS. 3A-3F show the predicted Lp(a) levels as a percentage of baseline for Q3M dosing of olpasiran at doses of 10 mg, 30 mg, 50 mg, 75 mg, 150 mg, and 225 mg for subjects with baseline Lp(a) levels of ≥150 nmol/L. The model predicts that doses of 10 mg or higher will suppress Lp(a) levels to 80% or greater throughout the 3-month dosing interval. Table 4 below shows the predicted proportion of subjects achieving a reduction of at least 80% from baseline in Lp(a) levels with different doses of olpasiran administered once every 3 months (Q3M dosing) at each dosing interval, whereas Table 5 shows the predicted proportion of subjects achieving absolute Lp(a) levels of 50 nmol/L or less with the same olpasiran dosing regimens.

TABLE 4
Predicted Proportion of Subjects Achieving 80% or Greater
Reduction in Lp(a) with Q3M Dosing of Olpasiran

| | Predicted % of Subjects with ≥80% Lp(a) Reduction | | | |
|---|---|---|---|---|
| | Median (95% prediction interval) over 200 simulated trials* | | | |
| Dose (mg) | 3 Months | 6 Months | 9 Months | 12 Months |
| 4 | 8 (2-14) | 10 (4-20) | 12 (4-22) | 12 (6-20) |
| 6 | 16 (6-24) | 20 (14-30) | 22 (12-30) | 22 (14-32) |
| 9 | 28 (16-38) | 34 (24-44) | 36 (24-46) | 38 (28-48) |
| 10 | 32 (18-40) | 40 (26-50) | 42 (30-54) | 42 (32-54) |
| 30 | 74 (62-82) | 78 (68-86) | 80 (68-86) | 78 (70-88) |
| 50 | 86 (78-92) | 88 (80-94) | 90 (82-96) | 90 (82-96) |
| 75 | 92 (84-96) | 92 (88-98) | 94 (88-100) | 94 (88-100) |
| 150 | 96 (92-100) | 96 (92-100) | 98 (92-100) | 98 (92-100) |
| 225 | 98 (94-100) | 98 (94-100) | 98 (94-100) | 98 (94-100) |

*50 subjects per trial

TABLE 5
Predicted Proportion of Subjects Achieving Absolute Lp(a)
Levels of 50 nmol/L or less with Q3M Dosing of Olpasiran

| | Predicted % of Subjects Achieving | | | |
|---|---|---|---|---|
| | Absolute Lp(a) Levels ≤50 nmol/L | | | |
| | Median (95% prediction interval) over 200 simulated trials* | | | |
| Dose (mg) | 3 Months | 6 Months | 9 Months | 12 Months |
| 4 | 10 (2-16) | 14 (6-22) | 14 (6-22) | 14 (6-22) |
| 6 | 18 (10-26) | 22 (12-32) | 24 (16-32) | 24 (14-34) |
| 9 | 28 (20-38) | 36 (24-46) | 36 (26-48) | 36 (26-50) |
| 10 | 32 (22-42) | 38 (28-50) | 40 (30-52) | 41 (30-52) |
| 30 | 70 (58-78) | 74 (62-84) | 76 (66-84) | 76 (64-84) |
| 50 | 82 (72-90) | 84 (76-92) | 86 (76-94) | 86 (76-94) |
| 75 | 88 (80-96) | 90 (82-98) | 90 (84-98) | 90 (84-96) |
| 150 | 94 (88-98) | 96 (90-100) | 96 (90-100) | 96 (90-100) |
| 225 | 96 (92-100) | 96 (92-100) | 96 (92-100) | 96 (92-100) |

*50 subjects per trial

Model simulations based on phase 1 data predict that a dose of 10 mg of olpasiran administered quarterly (Q3M) will result in about 42% of subjects having baseline Lp(a) levels of ≥150 nmol/L achieving at least 80% reduction in Lp(a) from baseline by month 12. Doses of 75 mg or greater of olpasiran administered quarterly are predicted to provide 80% or greater Lp(a) suppression in at least 90% of subjects having baseline Lp(a) levels of ≥150 nmol/L as early as 3 months after receiving a single dose of olpasiran. Similar proportions of subjects were predicted to achieve absolute Lp(a) values of 50 nmol/L or less with these same dosing regimens.

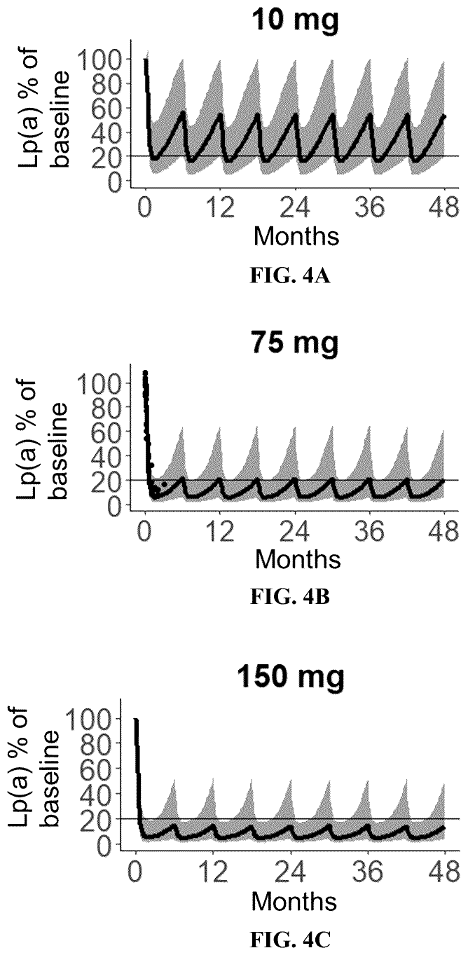

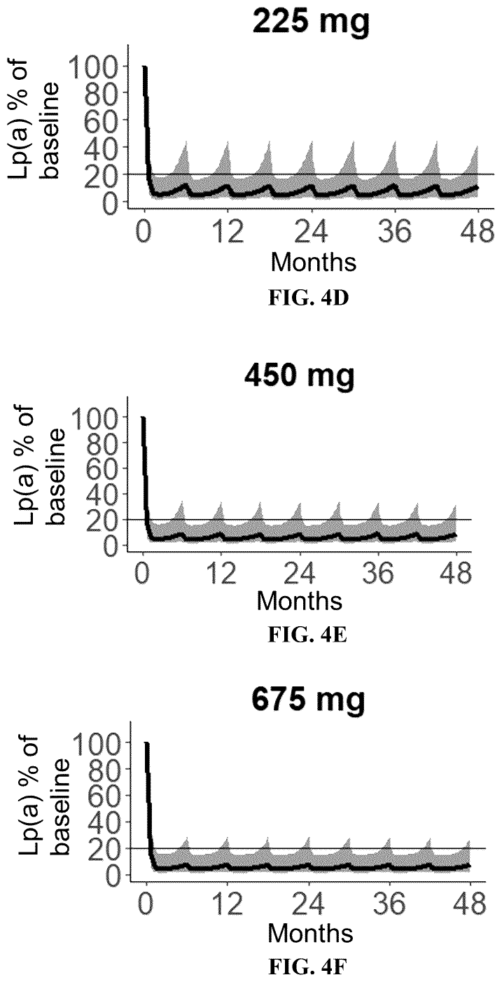

Simulations were also conducted to model biannual (Q6M) dosing regimens of olpasiran. FIGS. 4A-4F show the predicted Lp(a) levels as a percentage of baseline for Q6M dosing of olpasiran at doses of 10 mg, 75 mg, 150 mg, 225 mg, 450 mg, and 675 mg for subjects with baseline Lp(a) levels of ≥150 nmol/L. The model predicts that doses of at least 75 mg will suppress Lp(a) levels to 80% or greater throughout the 6-month dosing interval. Table 6 below shows the predicted proportion of subjects achieving a reduction of at least 80% from baseline in Lp(a) levels with different doses of olpasiran administered once every 6 months (Q6M dosing) at each dosing interval, whereas Table 7 shows the predicted proportion of subjects achieving absolute Lp(a) levels of 50 nmol/L or less with the same olpasiran dosing regimens.

TABLE 6
Predicted Proportion of Subjects Achieving 80% or Greater
Reduction in Lp(a) with Q6M Dosing of Olpasiran

| | Predicted % of Subjects with ≥80% Lp(a) Reduction | | | |
|---|---|---|---|---|
| | Median (95% prediction interval) over 200 simulated trials* | | | |
| Dose (mg) | 12 Months | 24 Months | 36 Months | 48 Months |
| 10 | 4 (2-8) | 4 (2-9) | 4 (2-8) | 4 (2-8) |
| 30 | 20 (12-30) | 20 (12-28) | 20 (12-30) | 20 (12-30) |
| 50 | 34 (24-46) | 36 (26-44) | 34 (24-46) | 34 (26-44) |
| 75 | 48 (36-60) | 46 (36-60) | 46 (36-60) | 47 (36-58) |
| 150 | 66 (56-76) | 67 (56-78) | 68 (56-78) | 66 (58-78) |
| 225 | 76 (66-86) | 76 (66-86) | 76 (68-86) | 76 (68-86) |
| 300 | 82 (72-90) | 82 (72-90) | 82 (72-90) | 82 (72-90) |
| 450 | 88 (78-94) | 88 (78-94) | 88 (78-94) | 88 (78-94) |
| 675 | 92 (84-98) | 92 (84-98) | 92 (84-98) | 92 (84-98) |

*50 subjects per trial

TABLE 7
Predicted Proportion of Subjects Achieving Absolute Lp(a)
Levels of 50 nmol/L or less with Q6M Dosing of Olpasiran

| | Predicted % of Subjects Achieving | | | |
|---|---|---|---|---|
| | Absolute Lp(a) Levels ≤50 nmol/L | | | |
| | Median (95% prediction interval) over 200 simulated trials* | | | |
| Dose (mg) | 12 Months | 24 Months | 36 Months | 48 Months |
| 10 | 4 (2-10) | 4 (2-10) | 5 (2-10) | 4 (2-10) |
| 30 | 22 (12-30) | 22 (12-30) | 22 (14-32) | 22 (14-32) |
| 50 | 34 (24-46) | 34 (24-46) | 34 (24-46) | 34 (24-46) |
| 75 | 46 (36-58) | 46 (34-58) | 46 (36-56) | 46 (36-58) |
| 150 | 64 (52-74) | 64 (54-74) | 64 (52-74) | 64 (54-74) |
| 225 | 72 (62-84) | 74 (64-82) | 72 (64-82) | 74 (62-82) |
| 300 | 78 (68-86) | 78 (68-88) | 78 (70-88) | 78 (68-88) |
| 450 | 84 (76-92) | 84 (76-92) | 84 (76-94) | 84 (76-92) |
| 675 | 88 (80-96) | 88 (80-96) | 88 (82-96) | 88 (80-96) |

*50 subjects per trial

The modeling data show that a dose of at least 75 mg of olpasiran administered once every six months (Q6M) is predicted to reduce Lp(a) levels by at least 80% from baseline in about 50% of subjects having baseline Lp(a) levels of ≥150 nmol/L after only two doses (i.e. after 12 months of treatment). The same proportion of patients is also predicted to achieve absolute Lp(a) levels less than 50 nmol/L with 75 mg of olpasiran administered once every six months after 1 year of treatment. A dose of 225 mg of olpasiran administered once every six months is predicted to suppress Lp(a) levels by at least 80% in about 76% of subjects following 1 year of treatment, whereas doses of 450 mg or greater administered once every six months are predicted to suppress Lp(a) levels greater than this threshold in about 90% of subjects following 1 year of treatment.

Based on recent mendelian randomization studies, reductions in Lp(a) levels of 80% or greater from baseline are expected to result in clinically meaningful cardiovascular benefit in patients with atherosclerotic cardiovascular disease (Burgess et al., JAMA Cardiol., Vol. 3:619-627, 2018; Lamina et al., JAMA Cardiol., Vol. 4: 575-579, 2019; and Madsen et al., Arterioscler. Thromb. Vasc. Biol., Vol. 40:255-266, 2020). Thus, the olpasiran PK/PD modeling was focused on identification of dosing regimens of olpasiran that could reduce Lp(a) levels greater than this threshold. The results of the olpasiran PK/PD modeling and simulation described in this Example indicate that:
a dose of 10 mg administered once every 3 months or once every 12 weeks provides ≥80% Lp(a) reduction in approximately half (42%) of subjects with baseline Lp(a) levels of ≥150 nmol/L by month 12 and provides median Lp(a) % reductions from baseline of about 77% at months 6 and 12;
a dose of 75 mg administered once every 3 months or once every 12 weeks is anticipated to provide ≥80% Lp(a) reduction from baseline within 2 to 3 doses in the majority (94%) of subjects and approximately 90% of subjects are expected to achieve absolute Lp(a) concentrations of 50 nmol/L or less with this dosing regimen;
a dose of 225 mg administered once every 3 months or once every 12 weeks is expected to provide ≥80% Lp(a) reduction in 98% of subjects and reduce Lp(a) levels to an absolute concentration of 50 nmol/L or less in 96% of subjects;
A dosing frequency of once every 3 months or once every 12 weeks for doses of 10 mg or greater result in suppression of Lp(a) levels below 20% of baseline throughout the entire 3-month dosing interval in the majority (≥90%) of subjects with baseline Lp(a) levels of 150 nmol/L or greater; and
A dose of 225 mg administered once every six months or once every 24 weeks will result in median Lp(a) reductions from baseline of 88% and with approximately 74% of subjects achieving absolute Lp(a) concentrations of 50 nmol/L or less.

### Example 3. A Double-Blind, Randomized, Placebo-Controlled Phase 2 Study to Evaluate Efficacy, Safety, and Tolerability of Olpasiran in Subjects with Elevated Lipoprotein(a)

The primary objective of this phase 2 study is to evaluate the effect of subcutaneous administration of olpasiran once every 12 weeks (Q12W) compared to placebo on percent change from baseline in Lp(a) levels after 36 weeks of treatment in subjects with atherosclerotic cardiovascular disease and elevated Lp(a). Secondary objectives of the study include the effects of olpasiran administered subcutaneously Q12W as compared with placebo on the percent change from baseline in: (i) Lp(a) levels after 48 weeks of treatment, (ii) low-density lipoprotein cholesterol (LDL-C) levels after 36 and 48 weeks of treatment, and (iii) apolipoprotein B (ApoB) levels after 36 and 48 weeks of treatment, and characterization of pharmacokinetic properties of olpasiran. Administration of olpasiran once every 24 weeks (Q24W) is also evaluated.

Approximately 240 subjects are randomized in a 1:1:1:1:1 ratio, with 4 arms being treated with olpasiran and 1 arm with placebo. The randomization is stratified by screening Lp(a)≤200 nmol/L vs.>200 nmol/L and by region (Japan vs. Non-Japan). The study treatment period is 48 weeks with doses at day 1, week 12, week 24, and week 36. After week 48 there is an extended safety follow-up without further dosing of olpasiran or placebo for ≥40 weeks and Lp(a) returns to 80% of baseline, whichever is later. Subjects remain on standard of care (including stable lipid-altering therapy) per their local guidelines during the treatment period and extended safety follow-up period.

After signing informed consent, subjects enter the screening phase (up to 4 weeks), during which eligibility of the subjects is assessed. Eligible subjects include adults 18 to 80 years of age with atherosclerotic cardiovascular disease having an Lp(a)>150 nmol/L during screening. Specifically, subjects are enrolled in the study if they meet all of the following key inclusion criteria:
Age 18 to 80 years
Lp(a)>150 nmol/L during screening by central laboratory
Atherosclerotic cardiovascular disease based on one of the following:
   History of coronary revascularization with percutaneous coronary intervention (PCI) or coronary artery bypass grafting (CABG);
   Diagnosis of coronary artery disease with or without prior myocardial infarction;
   Diagnosis of atherosclerotic cerebrovascular disease; or
   Diagnosis of peripheral arterial disease
For subjects receiving lipid-altering therapy (not required for eligibility), lipid altering therapy, including statin dose, must remain stable per local guidelines for ≥4 weeks prior to and during screening
   Subjects are excluded from the study if they meet any of the following key exclusion criteria:
Severe renal dysfunction as defined as an estimated glomerular filtration rate (eGFR)<30 mL/min/1.73 m² during screening
History or clinical evidence of hepatic dysfunction as defined as aspartate aminotransferase (AST) or alanine aminotransferase (ALT)>3×upper limit of normal (ULN), or total bilirubin (TBL)>2×ULN during screening
Malignancy (except non-melanoma skin cancers, cervical in-situ carcinoma, breast ductal carcinoma in situ, or stage 1 prostate carcinoma) within the last 5 years prior to day 1
Uncontrolled hypertension at day 1, defined as an average systolic blood pressure of ≥160 mmHg or an average diastolic blood pressure of ≥100 mmHg at rest
Fasting triglycerides≥400 mg/dL (4.5 mmol/L) during screening
Type 1 diabetes or poorly controlled type 2 diabetes mellitus as determined by a glycated hemoglobin (HbA1c)≥8.5% as determined by central laboratory at screening

Subjects eligible for the study have a baseline Lp(a)>150 nmol/L. This threshold is based on available epidemiological data showing that Lp(a)>125 nmol/L is considered elevated from the general population data (Averna et al., Atheroscler Suppl., Vol. 26:16-24, 2017; Nordestgaard and Langsted, J. Lipid Res., Vol. 57:1953-75, 2016; Ohro-Melander, J Intern Med., Vol. 278:433-46, 2015; Leebmann et al., Circulation, Vol. 128:2567-2576, 2013). In addition, based on the degree of absolute Lp(a) reduction necessary to demonstrate a corresponding effect on cardiovascular events, the enrolled population needs to have high baseline Lp(a). Therefore, an enrollment criterion of Lp(a)>150 nmol/L results in a study population with a median Lp(a) of approximately 200 nmol/L and allows evaluation of the effectiveness and safety of olpasiran in subjects with very high Lp(a).

Eligible and enrolled subjects are randomized in a 1:1:1:1:1 ratio to one of the following five treatment groups, with approximately 48 subjects in each group:
Group 1: 10 mg olpasiran Q12W
Group 2: 75 mg olpasiran Q12W
Group 3: 225 mg olpasiran Q12W
Group 4: 225 mg olpasiran Q24W
Group 5: Placebo Q12W
   As described in Example 2, these olpasiran dosing regimens are predicted to suppress Lp(a) levels by at least 80% from baseline throughout the dosing interval (3 months or 6 months) in human subjects with baseline Lp(a) levels>150 nmol/L. Olpasiran is administered by subcutaneous injection once every 12 weeks (treatment groups 1 to 3) or once every 24 weeks (treatment group 4) at a dose of 10 mg, 75 mg, or 225 mg depending on assigned treatment group. Samples to assess serum Lp(a), LDL-C, and ApoB and other clinical laboratory analytes are collected from enrolled subjects during screening, prior to administration of the first dose of olpasiran, and at weeks 12, 24, 36, and 48 as well as at other various time points during the study. Blood samples are collected for measurement of serum concentrations of olpasiran at various time points during the study to assess olpasiran pharmacokinetic parameters.

Screening for Lp(a) is conducted at a central laboratory using either an approved or investigational turbidimetric immunoassay that is standardized to detect and quantitate Lp(a) particles independent of apo(a) isoform size, such as the Tina-quant® Lipoprotein (a) Gen. 2 assay available from Roche Diagnostics. The assay is validated for measurements in nmol/L of Lp(a) in serum samples with a limit of detection of 7 nmol/L and is standardized against the IFCC reference material SRM2B for nmol/L (Marcovina et al., Clin. Chem., Vol. 46: 1946-1967, 2000). Lipid panels as well as assays for other clinical analytes, such as ApoB, hemoglobin A1C, ALT, AST, and bilirubin, are conducted by a central laboratory using standard methods.

The primary analysis occurs when all randomized subjects have had the opportunity to complete the week 36 assessments or have early terminated. The end of treatment period analysis occurs when all subjects have had the opportunity to complete the week 48 assessments or have early terminated. Final analysis occurs after the last subject either completes the extended safety follow-up and has ended the study or has early terminated from the study. The primary endpoint (percent change from baseline in Lp(a) at week 36) is compared between groups using repeated measures linear effects model including terms of treatment group, stratification factor, scheduled visit, and the interaction of treatment with scheduled visit. Hochberg procedure is used to control the type I error for multiple comparisons between active and placebo arms. The secondary endpoints percent change from baseline in Lp(a) at week 48, in ApoB and LDL-C at week 36 and 48 is analyzed similarly as the primary endpoint. Safety endpoints (e.g. treatment emergent adverse events) are summarized descriptively. Baseline Lp(a) is defined as the mean of the two most recent non-missing Lp(a) values measured through the central laboratory prior to or on study day 1. If for any reason only one value is available then that value is used as baseline.

Lp(a) reductions of 80% or greater from baseline have been observed with single doses of olpasiran lasting for greater than 3 months (see Example 1) and it is expected that this level of sustained reduction in Lp(a) may result in clinically meaningful cardiovascular benefit in patients with atherosclerotic cardiovascular disease by reducing the risk of cardiovascular events. Recent mendelian randomization studies suggests that in individuals with very high baseline Lp(a) concentrations, reducing Lp(a) by 80% to 90% is expected to translate into a clinically meaningful reduction in the risk of cardiovascular events (Burgess et al., JAMA Cardiol., Vol. 3:619-627, 2018; Lamina et al., JAMA Cardiol., Vol. 4: 575-579, 2019; and Madsen et al., Arterioscler. Thromb. Vasc. Biol., Vol. 40:255-266, 2020). Thus, the results of this study are expected to show that olpasiran as compared to placebo will produce a significant percent reduction from baseline in Lp(a) in subjects with atherosclerotic cardiovascular disease and elevated Lp(a) at all doses tested. In particular, it is expected that olpasiran, at doses as low as 10 mg administered once every 12 weeks, will effectively reduce Lp(a) levels to less than 50 nmol/L in the majority of subjects, which is expected to result in a reduction of risk of cardiovascular events in such subjects. Olpasiran administered at a dose of 75 mg once every 12 weeks is anticipated to be a particularly effective dosage regimen based on the PK/PD modeling described in Example 2.

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize, or be able to ascertain using no more than routine experimentation, many equivalents to the specific embodiments of the invention described herein. Such equivalents are intended to be encompassed by the following claims.

What is claimed:

1. A method for treating, reducing, or preventing atherosclerosis in a patient in need thereof comprising administering to the patient an LPA RNAi construct at a dose from about 9 mg to about 675 mg at a dosing interval of at least 8 weeks, wherein the LPA RNAi construct comprises a sense strand comprising the sequence of SEQ ID NO: 1, an antisense strand comprising the sequence of SEQ ID NO: 2, and a targeting moiety comprising an asialoglycoprotein receptor ligand, wherein the targeting moiety is covalently attached to the 5′ end of the sense strand.

2. A method for reducing serum or plasma Lp(a) levels in a patient in need thereof comprising administering to the patient an LPA RNAi construct at a dose from about 9 mg to about 675 mg at a dosing interval of at least 8 weeks, wherein the LPA RNAi construct comprises a sense strand comprising the sequence of SEQ ID NO: 1, an antisense strand comprising the sequence of SEQ ID NO: 2, and a targeting moiety comprising an asialoglycoprotein receptor ligand, wherein the targeting moiety is covalently attached to the 5′ end of the sense strand.

3. The method of claim 2, wherein the patient is diagnosed with or at risk of a cardiovascular disease.

4. The method of claim 3, wherein the cardiovascular disease is coronary artery disease, carotid artery disease, peripheral artery disease, myocardial infarction, cerebrovascular disease, stroke, aortic valve stenosis, stable or unstable angina, atrial fibrillation, heart failure, hyperlipidemia, heterozygous familial hypercholesterolemia, or homozygous familial hypercholesterolemia.

5. The method of claim 2, wherein the patient is diagnosed with chronic kidney disease.

6. The method of claim 2, wherein the patient has a history of myocardial infarction.

7. The method of claim 2, wherein the patient is diagnosed with acute coronary syndrome.

8. A method for treating, reducing, or preventing a cardiovascular disease in a patient in need thereof comprising administering to the patient an LPA RNAi construct at a dose from about 9 mg to about 675 mg at a dosing interval of at least 8 weeks, wherein the LPA RNAi construct comprises a sense strand comprising the sequence of SEQ ID NO: 1, an antisense strand comprising the sequence of SEQ ID NO: 2, and a targeting moiety comprising an asialoglycoprotein receptor ligand, wherein the targeting moiety is covalently attached to the 5′ end of the sense strand.

9. The method of claim 8, wherein the cardiovascular disease is coronary artery disease, carotid artery disease, peripheral artery disease, myocardial infarction, cerebrovascular disease, stroke, aortic valve stenosis, stable or unstable angina, atrial fibrillation, heart failure, hyperlipidemia, heterozygous familial hypercholesterolemia, or homozygous familial hypercholesterolemia.

10. A method for reducing the risk of a cardiovascular event in a patient with atherosclerotic cardiovascular disease comprising administering to the patient an LPA RNAi construct at a dose from about 9 mg to about 675 mg at a dosing interval of at least 8 weeks, wherein the LPA RNAi construct comprises a sense strand comprising the sequence of SEQ ID NO: 1, an antisense strand comprising the sequence of SEQ ID NO: 2, and a targeting moiety comprising an asialoglycoprotein receptor ligand, wherein the targeting moiety is covalently attached to the 5′ end of the sense strand.

11. The method of claim 10, wherein the cardiovascular event is cardiovascular death, myocardial infarction, stroke, and/or coronary revascularization.

12. The method of claim 10 or 11, wherein the patient has a history of coronary revascularization, a history of coronary artery bypass grafting, a diagnosis of coronary artery disease, a diagnosis of atherosclerotic cerebrovascular disease, a diagnosis of peripheral artery disease, and/or a history of myocardial infarction.

13. The method of any one of claims 10 to 12, wherein the patient has experienced a myocardial infarction within 1 year prior to the first administration of the LPA RNAi construct.

14. The method of any one of claims 10 to 12, wherein the patient is hospitalized for acute coronary syndrome or unstable angina.

15. The method of any one of claims 1 to 14, wherein the patient has a serum or plasma Lp(a) level of about 70 nmol/L or greater prior to the first administration of the LPA RNAi construct.

16. The method of any one of claims 1 to 14, wherein the patient has a serum or plasma Lp(a) level of about 100 nmol/L or greater prior to the first administration of the LPA RNAi construct.

17. The method of any one of claims 1 to 14, wherein the patient has a serum or plasma Lp(a) level of about 125 nmol/L or greater prior to the first administration of the LPA RNAi construct.

18. The method of any one of claims 1 to 14, wherein the patient has a serum or plasma Lp(a) level of about 150 nmol/L or greater prior to the first administration of the LPA RNAi construct.

19. The method of any one of claims 1 to 14, wherein the patient has a serum or plasma Lp(a) level of about 175 nmol/L or greater prior to the first administration of the LPA RNAi construct.

20. The method of any one of claims 1 to 14, wherein the patient has a serum or plasma Lp(a) level of about 200 nmol/L or greater prior to the first administration of the LPA RNAi construct.

21. The method of any one of claims 1 to 14, wherein the patient has a serum or plasma Lp(a) level of about 225 nmol/L or greater prior to the first administration of the LPA RNAi construct.

22. The method of any one of claims 1 to 21, wherein the dosing interval is about 12 weeks.

23. The method of any one of claims 1 to 21, wherein the dosing interval is about 24 weeks.

24. The method of any one of claims 1 to 21, wherein the LPA RNAi construct is administered to the patient at a dose from about 10 mg to about 225 mg once every 12 weeks.

25. The method of claim 24, wherein the LPA RNAi construct is administered to the patient at a dose from about 50 mg to about 100 mg once every 12 weeks.

26. The method of claim 24, wherein the LPA RNAi construct is administered to the patient at a dose from about 150 mg to about 225 mg once every 12 weeks.

27. The method of claim 24, wherein the LPA RNAi construct is administered to the patient at a dose of about 75 mg once every 12 weeks.

28. The method of claim 24, wherein the LPA RNAi construct is administered to the patient at a dose of about 150 mg once every 12 weeks.

29. The method of claim 24, wherein the LPA RNAi construct is administered to the patient at a dose of about 225 mg once every 12 weeks.

30. The method of any one of claims 1 to 21, wherein the LPA RNAi construct is administered to the patient at a dose from about 225 mg to about 675 mg once every 24 weeks.

31. The method of claim 30, wherein the LPA RNAi construct is administered to the patient at a dose of about 225 mg once every 24 weeks.

32. The method of any one of claims 1 to 31, wherein the patient is receiving a lipid-lowering therapy.

33. The method of claim 32, wherein the lipid-lowering therapy is a statin, ezetimibe, a PCSK9 inhibitor, bempedoic acid, or combinations thereof.

34. The method of any one of claims 1 to 33, wherein the patient has a serum low-density lipoprotein cholesterol (LDL-C) level of about 100 mg/dL or less prior to the first administration of the LPA RNAi construct.

35. The method of any one of claims 1 to 33, wherein the patient has a serum low-density lipoprotein cholesterol (LDL-C) level of about 70 mg/dL or less prior to the first administration of the LPA RNAi construct.

36. The method of any one of claims 1 to 35, wherein the patient has an estimated glomerular filtration rate of about 30 mL/min/1.73 m 2 or greater prior to the first administration of the LPA RNAi construct.

37. The method of any one of claims 1 to 36, wherein the patient has an average systolic blood pressure less than about 160 mmHg and an average diastolic blood pressure of less than about 100 mmHg at rest prior to the first administration of the LPA RNAi construct.

38. The method of any one of claims 1 to 37, wherein the patient has a glycated hemoglobin A1c level less than about 8.5% prior to the first administration of the LPA RNAi construct.

39. The method of any one of claims 1 to 38, wherein the patient has a serum triglyceride level of less than about 400 mg/dL prior to the first administration of the LPA RNAi construct.

40. The method of any one of claims 1 to 39, wherein the sense strand of the LPA RNAi construct comprises or consists of the sequence of SEQ ID NO: 3 and the antisense strand of the LPA RNAi construct comprises or consists of the sequence of SEQ ID NO: 4.

41. The method of any one of claims 1 to 40, wherein the sense strand of the LPA RNAi construct comprises or consists of the sequence of modified nucleotides according to SEQ ID NO: 5 and the antisense strand of the LPA RNAi construct comprises or consists of the sequence of modified nucleotides according to SEQ ID NO: 6.

42. The method of any one of claims 1 to 41, wherein the targeting moiety of the LPA RNAi construct has the structure of:

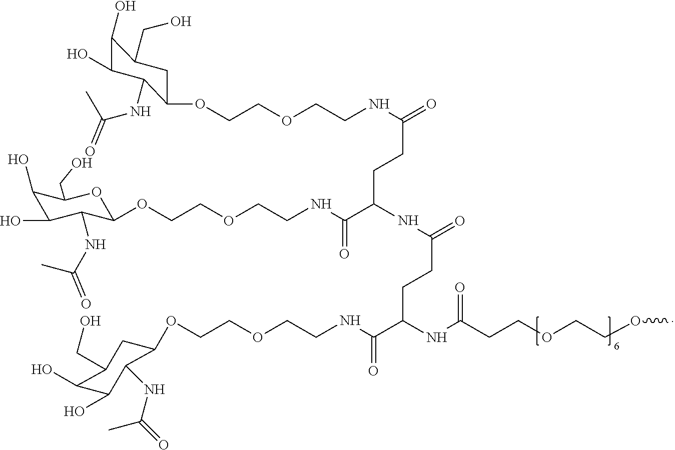

43. The method of any one of claims 1 to 42, wherein the LPA RNAi construct is olpasiran.

44. The method of any one of claims 1 to 43, wherein administration of the LPA RNAi construct reduces serum or plasma Lp(a) levels in the patient by greater than 50% for at least 12 weeks as compared to the patient's baseline serum or plasma Lp(a) levels.

45. The method of any one of claims 1 to 43, wherein administration of the LPA RNAi construct reduces serum or plasma Lp(a) levels in the patient by greater than 80% for at least 12 weeks as compared to the patient's baseline serum or plasma Lp(a) levels.

46. The method of any one of claims 1 to 43, wherein administration of the LPA RNAi construct reduces serum or plasma Lp(a) levels in the patient by greater than 90% for at least 12 weeks as compared to the patient's baseline serum or plasma Lp(a) levels.

47. The method of any one of claims 1 to 43, wherein administration of the LPA RNAi construct reduces serum or plasma Lp(a) levels in the patient to about 100 nmol/L or less.

48. The method of any one of claims 1 to 43, wherein administration of the LPA RNAi construct reduces serum or plasma Lp(a) levels in the patient to about 75 nmol/L or less.

49. The method of any one of claims 1 to 43, wherein administration of the LPA RNAi construct reduces serum or plasma Lp(a) levels in the patient to about 50 nmol/L or less.

50. The method of any one of claims 1 to 49, wherein the LPA RNAi construct is administered to the patient in a pharmaceutical composition comprising potassium phosphate and sodium chloride.

51. The method of any one of claims 1 to 50, wherein the LPA RNAi construct is administered to the patient by subcutaneous injection.

52. The method of claim 51, wherein the injection volume is about 1 mL or less.

53. An LPA RNAi construct for use in a method for treating, reducing, or preventing atherosclerosis in a patient in need thereof, wherein the method comprises administering to the patient the LPA RNAi construct at a dose from about 9 mg to about 675 mg at a dosing interval of at least 8 weeks, wherein the LPA RNAi construct comprises a sense strand comprising the sequence of SEQ ID NO: 1, an antisense strand comprising the sequence of SEQ ID NO: 2, and a targeting moiety comprising an asialoglycoprotein receptor ligand, wherein the targeting moiety is covalently attached to the 5′ end of the sense strand.

54. An LPA RNAi construct for use in a method for reducing serum or plasma Lp(a) levels in a patient in need thereof, wherein the method comprises administering to the patient the LPA RNAi construct at a dose from about 9 mg to about 675 mg at a dosing interval of at least 8 weeks, wherein the LPA RNAi construct comprises a sense strand comprising the sequence of SEQ ID NO: 1, an antisense strand comprising the sequence of SEQ ID NO: 2, and a targeting moiety comprising an asialoglycoprotein receptor ligand, wherein the targeting moiety is covalently attached to the 5′ end of the sense strand.

55. The LPA RNAi construct for use according to claim 54, wherein the patient is diagnosed with or at risk of a cardiovascular disease.

56. The LPA RNAi construct for use according to claim 55, wherein the cardiovascular disease is coronary artery disease, carotid artery disease, peripheral artery disease, myocardial infarction, cerebrovascular disease, stroke, aortic valve stenosis, stable or unstable angina, atrial fibrillation, heart failure, hyperlipidemia, heterozygous familial hypercholesterolemia, or homozygous familial hypercholesterolemia.

57. The LPA RNAi construct for use according to claim 54, wherein the patient is diagnosed with chronic kidney disease.

58. The LPA RNAi construct for use according to f claim 54, wherein the patient has a history of myocardial infarction.

59. The LPA RNAi construct for use according to claim 54, wherein the patient is diagnosed with acute coronary syndrome.

60. An LPA RNAi construct for use in a method for treating, reducing, or preventing a cardiovascular disease in a patient in need thereof, wherein the method comprises administering to the patient the LPA RNAi construct at a dose from about 9 mg to about 675 mg at a dosing interval of at least 8 weeks, wherein the LPA RNAi construct comprises a sense strand comprising the sequence of SEQ ID NO: 1, an antisense strand comprising the sequence of SEQ ID NO: 2, and a targeting moiety comprising an asialoglycoprotein receptor ligand, wherein the targeting moiety is covalently attached to the 5′ end of the sense strand.

61. The LPA RNAi construct for use according to claim 60, wherein the cardiovascular disease is coronary artery disease, carotid artery disease, peripheral artery disease, myocardial infarction, cerebrovascular disease, stroke, aortic valve stenosis, stable or unstable angina, atrial fibrillation, heart failure, hyperlipidemia, heterozygous familial hypercholesterolemia, or homozygous familial hypercholesterolemia.

62. An LPA RNAi construct for use in a method for reducing the risk of a cardiovascular event in a patient with atherosclerotic cardiovascular disease, wherein the method comprises administering to the patient the LPA RNAi construct at a dose from about 9 mg to about 675 mg at a dosing interval of at least 8 weeks, wherein the LPA RNAi construct comprises a sense strand comprising the sequence of SEQ ID NO: 1, an antisense strand comprising the sequence of SEQ ID NO: 2, and a targeting moiety comprising an asialoglycoprotein receptor ligand, wherein the targeting moiety is covalently attached to the 5′ end of the sense strand.

63. The LPA RNAi construct for use according to claim 62, wherein the cardiovascular event is cardiovascular death, myocardial infarction, stroke, and/or coronary revascularization.

64. The LPA RNAi construct for use according to claim 62 or 63, wherein the patient has a history of coronary revascularization, a history of coronary artery bypass grafting, a diagnosis of coronary artery disease, a diagnosis of atherosclerotic cerebrovascular disease, a diagnosis of peripheral artery disease, and/or a history of myocardial infarction.

65. The LPA RNAi construct for use according to any one of claims 62 to 64, wherein the patient has experienced a myocardial infarction within 1 year prior to the first administration of the LPA RNAi construct.

66. The LPA RNAi construct for use according to any one of claims 62 to 64, wherein the patient is hospitalized for acute coronary syndrome or unstable angina.

67. The LPA RNAi construct for use according to any one of claims 53 to 66, wherein the patient has a serum or plasma Lp(a) level of about 70 nmol/L or greater prior to the first administration of the LPA RNAi construct.

68. The LPA RNAi construct for use according to any one of claims 53 to 66, wherein the patient has a serum or plasma Lp(a) level of about 100 nmol/L or greater prior to the first administration of the LPA RNAi construct.

69. The LPA RNAi construct for use according to any one of claims 53 to 66, wherein the patient has a serum or plasma Lp(a) level of about 125 nmol/L or greater prior to the first administration of the LPA RNAi construct.

70. The LPA RNAi construct for use according to any one of claims 53 to 66, wherein the patient has a serum or plasma Lp(a) level of about 150 nmol/L or greater prior to the first administration of the LPA RNAi construct.

71. The LPA RNAi construct for use according to any one of claims 53 to 66, wherein the patient has a serum or plasma Lp(a) level of about 175 nmol/L or greater prior to the first administration of the LPA RNAi construct.

72. The LPA RNAi construct for use according to any one of claims 53 to 66, wherein the patient has a serum or plasma Lp(a) level of about 200 nmol/L or greater prior to the first administration of the LPA RNAi construct.

73. The LPA RNAi construct for use according to any one of claims 53 to 66, wherein the patient has a serum or plasma Lp(a) level of about 225 nmol/L or greater prior to the first administration of the LPA RNAi construct.

74. The LPA RNAi construct for use according to any one of claims 53 to 73, wherein the dosing interval is about 12 weeks.

75. The LPA RNAi construct for use according to any one of claims 53 to 73, wherein the dosing interval is about 24 weeks.

76. The LPA RNAi construct for use according to any one of claims 53 to 73, wherein the LPA RNAi construct is administered to the patient at a dose from about 10 mg to about 225 mg once every 12 weeks.

77. The LPA RNAi construct for use according to claim 76, wherein the LPA RNAi construct is administered to the patient at a dose from about 50 mg to about 100 mg once every 12 weeks.

78. The LPA RNAi construct for use according to claim 76, wherein the LPA RNAi construct is administered to the patient at a dose from about 150 mg to about 225 mg once every 12 weeks.

79. The LPA RNAi construct for use according to claim 76, wherein the LPA RNAi construct is administered to the patient at a dose of about 75 mg once every 12 weeks.

80. The LPA RNAi construct for use according to claim 76, wherein the LPA RNAi construct is administered to the patient at a dose of about 150 mg once every 12 weeks.

81. The LPA RNAi construct for use according to claim 76, wherein the LPA RNAi construct is administered to the patient at a dose of about 225 mg once every 12 weeks.

82. The LPA RNAi construct for use according to any one of claims 53 to 73, wherein the LPA RNAi construct is administered to the patient at a dose from about 225 mg to about 675 mg once every 24 weeks.

83. The LPA RNAi construct for use according to claim 82, wherein the LPA RNAi construct is administered to the patient at a dose of about 225 mg once every 24 weeks.

84. The LPA RNAi construct for use according to any one of claims 53 to 83, wherein the patient is receiving a lipid-lowering therapy.

85. The LPA RNAi construct for use according to claim 84, wherein the lipid-lowering therapy is a statin, ezetimibe, a PCSK9 inhibitor, bempedoic acid, or combinations thereof.

86. The LPA RNAi construct for use according to any one of claims 53 to 85, wherein the patient has a serum low-density lipoprotein cholesterol (LDL-C) level of about 100 mg/dL or less prior to the first administration of the LPA RNAi construct.

87. The LPA RNAi construct for use according to any one of claims 53 to 85, wherein the patient has a serum low-density lipoprotein cholesterol (LDL-C) level of about 70 mg/dL or less prior to the first administration of the LPA RNAi construct.

88. The LPA RNAi construct for use according to any one of claims 53 to 87, wherein the patient has an estimated glomerular filtration rate of about 30 mL/min/1.73 m 2 or greater prior to the first administration of the LPA RNAi construct.

89. The LPA RNAi construct for use according to any one of claims 53 to 88, wherein the patient has an average systolic blood pressure less than about 160 mmHg and an average diastolic blood pressure of less than about 100 mmHg at rest prior to the first administration of the LPA RNAi construct.

90. The LPA RNAi construct for use according to any one of claims 53 to 89, wherein the patient has a glycated hemoglobin A1c level less than about 8.5% prior to the first administration of the LPA RNAi construct.

91. The LPA RNAi construct for use according to any one of claims 53 to 90, wherein the patient has a serum triglyceride level of less than about 400 mg/dL prior to the first administration of the LPA RNAi construct.

92. The LPA RNAi construct for use according to any one of claims 53 to 91, wherein the sense strand of the LPA RNAi construct comprises or consists of the sequence of SEQ ID NO: 3 and the antisense strand of the LPA RNAi construct comprises or consists of the sequence of SEQ ID NO: 4.

93. The LPA RNAi construct for use according to any one of claims 53 to 92, wherein the sense strand of the LPA RNAi construct comprises or consists of the sequence of modified nucleotides according to SEQ ID NO: 5 and the antisense strand of the LPA RNAi construct comprises or consists of the sequence of modified nucleotides according to SEQ ID NO: 6.

94. The LPA RNAi construct for use according to any one of claims 53 to 93, wherein the targeting moiety of the LPA RNAi construct has the structure of:

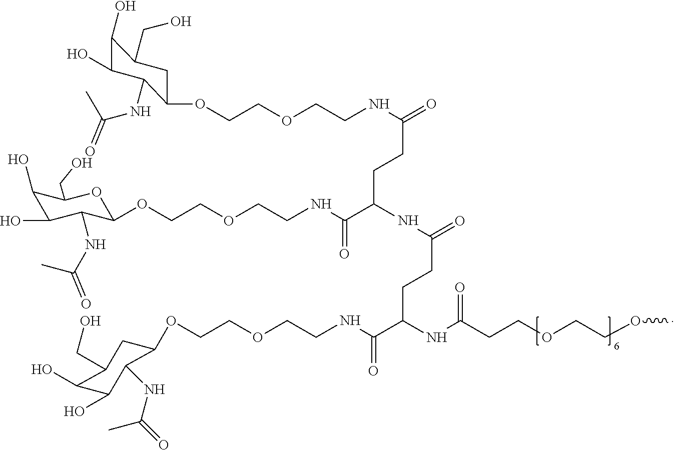

95. The LPA RNAi construct for use according to any one of claims 53 to 94, wherein the LPA RNAi construct is olpasiran.

96. The LPA RNAi construct for use according to any one of claims 53 to 95, wherein administration of the LPA RNAi construct reduces serum or plasma Lp(a) levels in the patient by greater than 50% for at least 12 weeks as compared to the patient's baseline serum or plasma Lp(a) levels.

97. The LPA RNAi construct for use according to any one of claims 53 to 95, wherein administration of the LPA RNAi construct reduces serum or plasma Lp(a) levels in the patient by greater than 80% for at least 12 weeks as compared to the patient's baseline serum or plasma Lp(a) levels.

98. The LPA RNAi construct for use according to any one of claims 53 to 95, wherein administration of the LPA RNAi construct reduces serum or plasma Lp(a) levels in the patient by greater than 90% for at least 12 weeks as compared to the patient's baseline serum or plasma Lp(a) levels.

99. The LPA RNAi construct for use according to any one of claims 53 to 95, wherein administration of the LPA RNAi construct reduces serum or plasma Lp(a) levels in the patient to about 100 nmol/L or less.

100. The LPA RNAi construct for use according to any one of claims 53 to 95, wherein administration of the LPA RNAi construct reduces serum or plasma Lp(a) levels in the patient to about 75 nmol/L or less.

101. The LPA RNAi construct for use according to any one of claims 53 to 95, wherein administration of the LPA RNAi construct reduces serum or plasma Lp(a) levels in the patient to about 50 nmol/L or less.

102. The LPA RNAi construct for use according to any one of claims 53 to 101, wherein the LPA RNAi construct is administered to the patient in a pharmaceutical composition comprising potassium phosphate and sodium chloride.

103. The LPA RNAi construct for use according to any one of claims 53 to 102, wherein the LPA RNAi construct is administered to the patient by subcutaneous injection.

104. The LPA RNAi construct for use according to claim 103, wherein the injection volume is about 1 mL or less.

105. Use of an LPA RNAi construct for preparation of a medicament for treating, reducing, or preventing atherosclerosis in a patient in need thereof, wherein the medicament is administered or formulated for administration at a dose from about 9 mg to about 675 mg at a dosing interval of at least 8 weeks, wherein the LPA RNAi construct comprises a sense strand comprising the sequence of SEQ ID NO: 1, an antisense strand comprising the sequence of SEQ ID NO: 2, and a targeting moiety comprising an asialoglycoprotein receptor ligand, wherein the targeting moiety is covalently attached to the 5′ end of the sense strand.

106. Use of an LPA RNAi construct for preparation of a medicament for reducing serum or plasma Lp(a) levels in a patient in need thereof, wherein the medicament is administered or formulated for administration at a dose from about 9 mg to about 675 mg at a dosing interval of at least 8 weeks, wherein the LPA RNAi construct comprises a sense strand comprising the sequence of SEQ ID NO: 1, an antisense strand comprising the sequence of SEQ ID NO: 2, and a targeting moiety comprising an asialoglycoprotein receptor ligand, wherein the targeting moiety is covalently attached to the 5′ end of the sense strand.

107. The use of claim 106, wherein the patient is diagnosed with or at risk of a cardiovascular disease.

108. The use of claim 107, wherein the cardiovascular disease is coronary artery disease, carotid artery disease, peripheral artery disease, myocardial infarction, cerebrovascular disease, stroke, aortic valve stenosis, stable or unstable angina, atrial fibrillation, heart failure, hyperlipidemia, heterozygous familial hypercholesterolemia, or homozygous familial hypercholesterolemia.

109. The use of claim 106, wherein the patient is diagnosed with chronic kidney disease.

110. The use of claim 106, wherein the patient has a history of myocardial infarction.

111. The use of claim 106, wherein the patient is diagnosed with acute coronary syndrome.

112. Use of an LPA RNAi construct for preparation of a medicament for treating, reducing, or preventing a cardiovascular disease in a patient in need thereof, wherein the medicament is administered or formulated for administration at a dose from about 9 mg to about 675 mg at a dosing interval of at least 8 weeks, wherein the LPA RNAi construct comprises a sense strand comprising the sequence of SEQ ID NO: 1, an antisense strand comprising the sequence of SEQ ID NO: 2, and a targeting moiety comprising an asialoglycoprotein receptor ligand, wherein the targeting moiety is covalently attached to the 5′ end of the sense strand.

113. The use of claim 112, wherein the cardiovascular disease is coronary artery disease, carotid artery disease, peripheral artery disease, myocardial infarction, cerebrovascular disease, stroke, aortic valve stenosis, stable or unstable angina, atrial fibrillation, heart failure, hyperlipidemia, heterozygous familial hypercholesterolemia, or homozygous familial hypercholesterolemia.

114. Use of an LPA RNAi construct for preparation of a medicament for reducing the risk of a cardiovascular event in a patient with atherosclerotic cardiovascular disease, wherein the medicament is administered or formulated for administration at a dose from about 9 mg to about 675 mg at a dosing interval of at least 8 weeks, wherein the LPA RNAi construct comprises a sense strand comprising the sequence of SEQ ID NO: 1, an antisense strand comprising the sequence of SEQ ID NO: 2, and a targeting moiety comprising an asialoglycoprotein receptor ligand, wherein the targeting moiety is covalently attached to the 5′ end of the sense strand.

115. The use of claim 114, wherein the cardiovascular event is cardiovascular death, myocardial infarction, stroke, and/or coronary revascularization.

116. The use of claim 114 or 115, wherein the patient has a history of coronary revascularization, a history of coronary artery bypass grafting, a diagnosis of coronary artery disease, a diagnosis of atherosclerotic cerebrovascular disease, a diagnosis of peripheral artery disease, and/or a history of myocardial infarction.

117. The use of any one of claims 114 to 116, wherein the patient has experienced a myocardial infarction within 1 year prior to the first administration of the medicament.

118. The use of any one of claims 114 to 116, wherein the patient is hospitalized for acute coronary syndrome or unstable angina.

119. The use of any one of claims 105 to 118, wherein the patient has a serum or plasma Lp(a) level of about 70 nmol/L or greater prior to the first administration of the medicament.

120. The use of any one of claims 105 to 118, wherein the patient has a serum or plasma Lp(a) level of about 100 nmol/L or greater prior to the first administration of the medicament.

121. The use of any one of claims 105 to 118, wherein the patient has a serum or plasma Lp(a) level of about 125 nmol/L or greater prior to the first administration of the medicament.

122. The use of any one of claims 105 to 118, wherein the patient has a serum or plasma Lp(a) level of about 150 nmol/L or greater prior to the first administration of the medicament.

123. The use of any one of claims 105 to 118, wherein the patient has a serum or plasma Lp(a) level of about 175 nmol/L or greater prior to the first administration of the medicament.

124. The use of any one of claims 105 to 118, wherein the patient has a serum or plasma Lp(a) level of about 200 nmol/L or greater prior to the first administration of the medicament.

125. The use of any one of claims 105 to 118, wherein the patient has a serum or plasma Lp(a) level of about 225 nmol/L or greater prior to the first administration of the medicament.

126. The use of any one of claims 105 to 125, wherein the dosing interval is about 12 weeks.

127. The use of any one of claims 105 to 125, wherein the dosing interval is about 24 weeks.

128. The use of any one of claims 105 to 125, wherein the medicament is administered or formulated for administration to the patient at a dose from about 10 mg to about 225 mg once every 12 weeks.

129. The use of claim 128, wherein the medicament is administered or formulated for administration to the patient at a dose from about 50 mg to about 100 mg once every 12 weeks.

130. The use of claim 128, wherein the medicament is administered or formulated for administration to the patient at a dose from about 150 mg to about 225 mg once every 12 weeks.

131. The use of claim 128, wherein the medicament is administered or formulated for administration to the patient at a dose of about 75 mg once every 12 weeks.

132. The use of claim 128, wherein the medicament is administered or formulated for administration to the patient at a dose of about 150 mg once every 12 weeks.

133. The use of claim 128, wherein the medicament is administered or formulated for administration to the patient at a dose of about 225 mg once every 12 weeks.

134. The use of any one of claims 105 to 125, wherein the medicament is administered or formulated for administration to the patient at a dose from about 225 mg to about 675 mg once every 24 weeks.

135. The use of claim 134, wherein the medicament is administered or formulated for administration to the patient at a dose of about 225 mg once every 24 weeks.

136. The use of any one of claims 105 to 135, wherein the patient is receiving a lipid-lowering therapy.

137. The use of claim 136, wherein the lipid-lowering therapy is a statin, ezetimibe, a PCSK9 inhibitor, bempedoic acid, or combinations thereof.

138. The use of any one of claims 105 to 137, wherein the patient has a serum low-density lipoprotein cholesterol (LDL-C) level of about 100 mg/dL or less prior to the first administration of the medicament.

139. The use of any one of claims 105 to 137, wherein the patient has a serum low-density lipoprotein cholesterol (LDL-C) level of about 70 mg/dL or less prior to the first administration of the medicament.

140. The use of any one of claims 105 to 139, wherein the patient has an estimated glomerular filtration rate of about 30 mL/min/1.73 m 2 or greater prior to the first administration of the medicament.

141. The use of any one of claims 105 to 140, wherein the patient has an average systolic blood pressure less than about 160 mmHg and an average diastolic blood pressure of less than about 100 mmHg at rest prior to the first administration of the medicament.

142. The use of any one of claims 105 to 141, wherein the patient has a glycated hemoglobin A1c level less than about 8.5% prior to the first administration of the medicament.

143. The use of any one of claims 105 to 142, wherein the patient has a serum triglyceride level of less than about 400 mg/dL prior to the first administration of the medicament.

144. The use of any one of claims 105 to 143, wherein the sense strand of the LPA RNAi construct comprises or consists of the sequence of SEQ ID NO: 3 and the antisense strand of the LPA RNAi construct comprises or consists of the sequence of SEQ ID NO: 4.

145. The use of any one of claims 105 to 144, wherein the sense strand of the LPA RNAi construct comprises or consists of the sequence of modified nucleotides according to SEQ ID NO: 5 and the antisense strand of the LPA RNAi construct comprises or consists of the sequence of modified nucleotides according to SEQ ID NO: 6.

146. The use of any one of claims 105 to 145, wherein the targeting moiety of the LPA RNAi construct has the structure of:

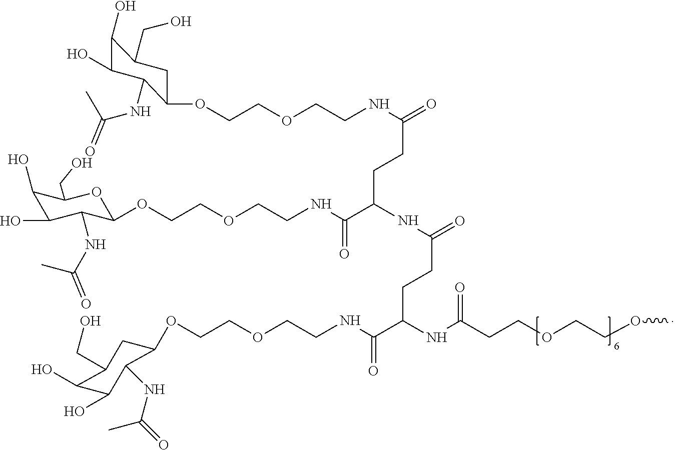

147. The use of any one of claims 105 to 146, wherein the LPA RNAi construct is olpasiran.

148. The use of any one of claims 105 to 147, wherein administration of the medicament reduces serum or plasma Lp(a) levels in the patient by greater than 50% for at least 12 weeks as compared to the patient's baseline serum or plasma Lp(a) levels.

149. The use of any one of claims 105 to 147, wherein administration of the medicament reduces serum or plasma Lp(a) levels in the patient by greater than 80% for at least 12 weeks as compared to the patient's baseline serum or plasma Lp(a) levels.

150. The use of any one of claims 105 to 147, wherein administration of the medicament reduces serum or plasma Lp(a) levels in the patient by greater than 90% for at least 12 weeks as compared to the patient's baseline serum or plasma Lp(a) levels.

151. The use of any one of claims 105 to 147, wherein administration of the medicament reduces serum or plasma Lp(a) levels in the patient to about 100 nmol/L or less.

152. The use of any one of claims 105 to 147, wherein administration of the medicament reduces serum or plasma Lp(a) levels in the patient to about 75 nmol/L or less.

153. The use of any one of claims 105 to 147, wherein administration of the medicament reduces serum or plasma Lp(a) levels in the patient to about 50 nmol/L or less.

154. The use of any one of claims 105 to 153, wherein the medicament is administered or formulated for administration to the patient in a pharmaceutical composition comprising potassium phosphate and sodium chloride.

155. The use of any one of claims 105 to 154, wherein the medicament is administered or formulated for administration to the patient by subcutaneous injection.

156. The use of claim 155, wherein the injection volume is about 1 mL or less.


SEQUENCE LISTING (12 sequences)

SEQ ID NO: 1 - RNA, 18 nt, Artificial Sequence
  Synthetic polynucleotide
gccccuuauuguuauacg

SEQ ID NO: 2 - RNA, 18 nt, Artificial Sequence
  Synthetic polynucleotide
cguauaacaauaaggggc

SEQ ID NO: 3 - RNA, 21 nt, Artificial Sequence
  Synthetic polynucleotide
cagccccuuauuguuauacga

SEQ ID NO: 4 - RNA, 21 nt, Artificial Sequence
  Synthetic polynucleotide
ucguauaacaauaaggggcug

SEQ ID NO: 5 - RNA, 21 nt, Artificial Sequence
  Synthetic modified polynucleotide
  modified_base (1)..(21): combination of 2′-fluoro, 2′-O-methyl, inverted deoxyadenosine, phosphorothioate linked ribonucleotides
cagccccuuauuguuauacga

SEQ ID NO: 6 - RNA, 21 nt, Artificial Sequence
  Synthetic modified polynucleotide
  modified_base (1)..(21): combination of 2′-fluoro, 2′-O-methyl, phosphorothioate linked ribonucleotides
ucguauaacaauaaggggcug

SEQ ID NO: 7 - RNA, 21 nt, Artificial Sequence
  Synthetic polynucleotide
gccccuuauuguuauacgauu

SEQ ID NO: 8 - RNA, 21 nt, Artificial Sequence
  Synthetic polynucleotide
ucguauaacaauaaggggcuu

SEQ ID NO: 9 - RNA, 21 nt, Artificial Sequence
  Synthetic modified polynucleotide
  modified_base (1)..(21): combination of 2′-fluoro, 2′-O-methyl, inverted abasic, phosphorothioate linked ribonucleotides
gccccuuauuguuauacgauu

SEQ ID NO: 10 - RNA, 21 nt, Artificial Sequence
  Synthetic modified polynucleotide
  modified_base (1)..(21): combination of 2′-fluoro, 2′-O-methyl, phosphorothioate linked ribonucleotides
ucguauaacaauaaggggcuu

SEQ ID NO: 11 - RNA, 21 nt, Artificial Sequence
  Synthetic modified polynucleotide
  modified_base (1)..(21): combination of 2′-fluoro, 2′-O-methyl, inverted abasic, phosphorothioate linked ribonucleotides
gccccuuauuguuauacgauu

SEQ ID NO: 12 - RNA, 21 nt, Artificial Sequence
  Synthetic modified polynucleotide
  modified_base (1)..(21): combination of 2′-fluoro, 2′-O-methyl, phosphorothioate linked ribonucleotides
ucguauaacaauaaggggcuu